SEQUENCE LISTING (113 sequences)

SEQ ID NO: 1 - protein, 1061 aa, Staphylococcus auricularis
MQENQQKQNYILGLDIGITSVGYGLIDSKTREVIDAGVRLFPEADSENNSNRRSKRGARRLKRRRIHRLNRVKDLLADYQMIDLNNVPKSTDPYTIRVKGLREPLTKEEFAIALLHIAKRRGLHNISVSMGDEEQDNELSTKQQLQKNAQQLQDKYVCELQLERLTNINKVRGEKNRFKTEDFVKEVKQLCETQRQYHNIDDQFIQQYIDLVSTRREYFEGPGNGSPYGWDGDLLKWYEKLMGRCTYFPEELRSVKYAYSADLFNALNDLNNLVVTRDDNPKLEYYEKYHIIENVFKQKKNPTLKQIAKEIGVQDYDIRGYRITKSGKPQFTSFKLYHDLKNIFEQAKYLEDVEMLDEIAKILTIYQDEISIKKALDQLPELLTESEKSQIAQLTGYTGTHRLSLKCIHIVIDELWESPENQMEIFTRLNLKPKKVEMSEIDSIPTTLVDEFILSPVVKRAFIQSIKVINAVINRFGLPEDIIIELAREKNSKDRRKFINKLQKQNEATRKKIEQLLAKYGNTNAKYMIEKIKLHDMQEGKCLYSLEAIPLEDLLSNPTHYEVDHIIPRSVSFDNSLNNKVLVKQSENSKKGNRTPYQYLSSNESKISYNQFKQHILNLSKAKDRISKKKRDMLLEERDINKFEVQKEFINRNLVDTRYATRELSNLLKTYFSTHDYAVKVKTINGGFTNHLRKVWDFKKHRNHGYKHHAEDALVIANADFLFKTHKALRRTDKILEQPGLEVNDTTVKVDTEEKYQELFETPKQVKNIKQFRDFKYSHRVDKKPNRQLINDTLYSTREIDGETYVVQTLKDLYAKDNEKVKKLFTERPQKILMYQHDPKTFEKLMTILNQYAEAKNPLAAYYEDKGEYVTKYAKKGNGPAIHKIKYIDKKLGSYLDVSNKYPETQNKLVKLSLKSFRFDIYKCEQGYKMVSIGYLDVLKKDNYYYIPKDKYEAEKQKKKIKESDLFVGSFYYNDLIMYEDELFRVIGVNSDINNLVELNMVDITYKDFCEVNNVTGEKRIKKTIGKRVVLIEKYTTDILGNLYKTPLPKKPQLIFKRGEL

SEQ ID NO: 2 - protein, 1055 aa, Staphylococcus haemolyticus
MKTDYILGLDIGITSVGYGIINYNDKSIIDAGVRLFPEANVENNEGRRSKRGARRLKRRRIHRLERVKQLLLDYKLLDSIDVIPQSTNPYEIRVRGLREKLTKDELVIALLHLAKRRGIHNIDVIDQEEDASNELSTKEQLSKNNLLLRDKSICEVLLERYNEGKVRGEKNRFKTSDIVNEIRQILETQKEVHHLDDSFIDKYIELVETRREYYEGPGEGSPYGWGADLKKWYEHLMGRCTYFPEELRSVKYAYSADLFNALNDLNNLVIQRDGLNKLEYHEKYHIIENVFKQKKKPTLKQISNEIGVNPEDIKGYRITKSGKENFTEFKLYHDLKKILKDQSILENIQLLDQIAEIITIYQDKESIKKELEQLTEPINEIDKESISDLAGYNGTHRLSLKCINLVLEELWHTSRNQMEIFAYLNIKPRKIDLQKANKIPKDMIDEFILSPVVKRTFGQAINVINKVIEKYGVPKDIIIELARENNTKDKQKFINELQKKNEKTRQRINEIIGKTGNQNAKRLVEKIRLHDEQEGKCLYSLESIPLEDLLNNPNHYEVDHIIPRSVSFDNSYQNKVLVKQTENSKKGNRTPYQYLNSGEAKLSYNQFKQHVLNLSKSKDRISKKKKEYLLEERDINKFEVQKEFINRNLVDTRYATRELTNYLKAYFSANDMDVKVKTINGSFTNHLRKVWDFNKERNHGYKHHAEDALIIANADFLFKENKKLKAANKVLEQPERKEIETKIEVNSDENYQDLFVIPQQVKEIKEFRDFKYSHRVDKKPNRQLINDTLYSTRVKDDDLYIISPIKNIYSKDNTDLKKHFNKNPEKFLMYQKDPKTFEKLEVIMKQYANEKNPLAKYHEETGEYLTKYSKTNNGPIVKSIKMFGKKVGNHLDVTNQYNNSRNKLVKLSFKSYRFDVYLTDKGYKFVSITYLDVLKKENYYYISEAKYDKLKLNKGIDDKAKFIGSFYYNDLIELDGEVYTVIGVNNDKNNVIELNLPEIRYKEYCEINNIKGSGRLRITIGKKVNSIRKLSTDVLGNRYYQSFAKKPQLVFKKGI

SEQ ID NO: 3 - protein, 1054 aa, Staphylococcus lugdunensis
MNQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIESKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 4 - protein, 1054 aa, Staphylococcus lutrae
MRNSYILGLDIGITSVGYGIIDRVTREVIDAGVRLFPEANVENNEGRRSKRGARRLKRRRIHRLNRIKQLLKNAGLLEGDVLPKSTNPYEIRVRGLRSPLTKDELVIALLHIAKRRGIHNINIVGDDEETDSTLSTTAQLKKNEKALKGQFVCELQLDRLANAHQVRGEKNRFKTEDIVKEVRALLQQQQNFHNIDNSFVEQYIALLESRRTYYEGPGEGSPYGWDGDIKKWYEMLMGYCTYFPEELRSVKYAYTADLFNALNDLNNLVITRDDNSKLTYAEKYHIIENVFKQKKVPTLKQIAKEIGVNESDIKGYRINKSEKPLFTSFKLYHDLKSVFSDPTKLEDIDLLDRIAVVLTMYQDAESMKAALNTFPEVFSEAEKEKLSALTGYAGTHRLSLKCMNLLIPDLWQTSLNQMELFVKLNLKPQKLDLSQCHQIPTQLVDEFILSPVVKRAFTQSIKVINAIIQKYGLPDDIIIELAREKNSADKRKFLNQLQKKNEKARHEINTLVAQYGQPNAKRLVEKITLHQQQEGKCLYSLKDIPLEQLLKQPYLYEVDHIIPRSVSFDNSMQNKVLVLAEENAKKGNQTPYQYLNSREASMTYPEFKQHILNLSKAKDRISKKKRNYLLEERDINKFDVQKDFINRNLVDTRYATRELASLLKAYFKTHELPVKVKTINGGFTHYLRKVWKFDKDRNKGYKHHAEDALIIANADFLFKNQTLNKIEAILNEPGREVESDTVKVQSEDNYQDLFENTKKAFAIKNFKDFKFSHRVDQKPNRQLVNDTLYSTREVNEDLYVVQTLKDIYSKDNKDVKRLFDKQPEKFLMFQHDPETFKKFELAMKQYAEEKNPLARYYEEQGYITKYAKKGDGPPVKSLKYIGKKVGKHLDVTGDYEDSNRKLVKLSLKSFRFDIYHTDKGYKMVPITYLDVQKKEKYYYIPTEKYEALKQEKGINQNAQFIGSFYYNDLIEFDGELYRVIGINNGDKNLVELDMVDIRYKEYCELNSITTTPRIVKTISPKTQSIEKYTTDILGNLYKAQPGKKPQFIFNKDED

SEQ ID NO: 5 - protein, 1056 aa, Artificial Sequence
  a fusion protein obtained by replacing the PI domain of SaCas9 with the PI domain of SauriCas9, wherein SauriCas9 is Staphylococcus auricularis Cas9
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNAATNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATAALMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKSFRFDIYKCEQGYKMVSIGYLDVLKKDNYYYIPKDKYEAEKQKKKIKESDLFVGSFYYNDLIMYEDELFRVIGVNSDINNLVELNMVDITYKDFCEVNNVTGEKRIKKTIGKRVVLIEKYTTDILGNLYKTPLPKKPQLIFKRGEL

SEQ ID NO: 6 - protein, 1055 aa, Artificial Sequence
  a fusion protein obtained by replacing the PI domain of SaCas9 with the PI domain of SepCas9, wherein SepCas9 is Staphylococcus epidermidis Cas9
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNAATNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATAALMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKNYRFDVYLTEKGYKFVTIAYLNVFKKDNYYYIPKDMYQELKAKKKIKDTDQFIASFYKNDLIKLNGDLYKIIGVNSDDRNIIELDYYDIKYKDYCEINNIKGEPRIKKTIGKKTESIEKLTTDVLGNLYLHSTEKAPQLIFKRGL

SEQ ID NO: 7 - protein, 1053 aa, Artificial Sequence
  a fusion protein obtained by replacing the PI domain of SaCas9 with the PI domain of SeqCas9, wherein SeqCas9 is Staphylococcus equorum Cas9
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNAATNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATAALMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLNVIKKENYYEVNSKCYEKAKKLKKISDQAEFIASFYNNDLIKIDGELYRVIGVNTDLINRIEVNMVDITYREYLENMNDKRSPRIFKTIASKTQSIKKYSTDILGTLYEVNSKKHPQMIMKG

SEQ ID NO: 8 - protein, 1055 aa, Artificial Sequence
  a fusion protein obtained by replacing the PI domain of SaCas9 with the PI domain of ShaCas9, wherein ShaCas9 is Staphylococcus haemolyticus Cas9
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNAATNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATAALMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKSYRFDVYLTDKGYKFVSITYLDVLKKENYYYISEAKYDKLKLNKGIDDKAKFIGSFYYNDLIELDGEVYTVIGVNNDKNNVIELNLPEIRYKEYCEINNIKGSGRLRITIGKKVNSIRKLSTDVLGNRYYQSFAKKPQLVFKKGI

SEQ ID NO: 9 - protein, 1055 aa, Artificial Sequence
  a fusion protein obtained by replacing the PI domain of SaCas9 with the PI domain of SlugCas9, wherein SlugCas9 is Staphylococcus lugdunensis Cas9
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNAATNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATAALMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 10 - protein, 1056 aa, Artificial Sequence
  a fusion protein obtained by replacing the PI domain of SaCas9 with the PI domain of SlutCas9, wherein SlutCas9 is Staphylococcus lutrae Cas9
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNAATNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATAALMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKSFRFDIYHTDKGYKMVPITYLDVQKKEKYYYIPTEKYEALKQEKGINQNAQFIGSFYYNDLIEFDGELYRVIGINNGDKNLVELDMVDIRYKEYCELNSITTTPRIVKTISPKTQSIEKYTTDILGNLYKAQPGKKPQFIFNKDED

SEQ ID NO: 11 - RNA, 101 nt, Artificial Sequence
  nucleotide sequence of sgRNA
  misc_feature (1)..(20): n=a or u or c or g
nnnnnnnnnnnnnnnnnnnnguuuuaguacucuggaaacagaaucuacuaaaacaaggcaaaaugccguguuuaucucgucaacuuguuggcgagauuuuu

SEQ ID NO: 12 - DNA, 25 bp, Artificial Sequence
  target DNA sequence
  misc_feature (1)..(23): n=a or t or c or g
nnnnnnnnnnnnnnnnnnnnnnngg

SEQ ID NO: 13 - DNA, 26 bp, Artificial sequence
  target DNA sequence
  misc_feature (1)..(23): n=a or t or c or g
  misc_feature (25)..(25): r=a or g
  misc_feature (26)..(26): m=a or c
nnnnnnnnnnnnnnnnnnnnnnngrm

SEQ ID NO: 14 - DNA, 25 bp, Artificial Sequence
  target DNA sequence
  misc_feature (1)..(23): n=a or g or t or c
  misc_feature (25)..(25): r=a or g
nnnnnnnnnnnnnnnnnnnnnnngr

SEQ ID NO: 15 - DNA, 26 bp, Artificial Sequence
  target DNA sequence
  misc_feature (1)..(23): n=a or t or c or g
  misc_feature (25)..(26): r=a or g
nnnnnnnnnnnnnnnnnnnnnnngrr

SEQ ID NO: 16 - DNA, 24 bp, Artificial Sequence
  Synthetic oligonucleotide
caccgctcggagatcatcattgcg

SEQ ID NO: 17 - DNA, 24 bp, Artificial Sequence
  Synthetic oligonucleotide
aaaccgcaatgatgatctccgagc

SEQ ID NO: 18 - DNA, 25 bp, artificial sequence
  nucleotide sequence of the target site
  misc_feature (21)..(25): n=a or t or c or g
gctcggagatcatcattgcgnnnnn

SEQ ID NO: 19 - DNA, 55 bp, Artificial Sequence
  primer sequence for PCR amplification
  misc_feature (34)..(37): n is a, c, g, or t
acactctttccctacacgacgctcttccgatctnnnngcgagaaaagccttgttt

SEQ ID NO: 20 - DNA, 51 bp, Artificial Sequence
  primer sequence for PCR amplification
actggagttcagacgtgtgctcttccgatctctgaacttgtggccgtttac

SEQ ID NO: 21 - DNA, 45 bp, Artificial Sequence
  primer sequence for PCR amplification
aatgatacggcgaccaccgagatctacactctttccctacacgac

SEQ ID NO: 22 - DNA, 52 bp, Artificial Sequence
  primer sequence for PCR amplification
caagcagaagacggcatacgagatcactgtgtgactggagttcagacgtgtg

SEQ ID NO: 23 - DNA, 3183 bp, Artificial Sequence
  humanized SauriCas9 gene sequence
atgcaggagaaccagcagaagcagaactacatcctgggcctggacatcggaatcaccagcgtcggctacggactgatcgatagcaagacaagagaagtgatcgacgccggcgttagactctttccagaagctgatagcgagaacaactccaaccgcagaagcaagcggggcgccagacggttaaaacggagaagaatccaccggctgaaccgggtcaaagacctgctcgctgattaccagatgatcgatcttaacaatgttcctaagagcaccgacccctacaccatcagagtgaagggcctccgggagcctctgacaaaagaagaattcgccatcgccctcctgcatatcgctaagagaagaggcctgcacaacatcagtgtgtccatgggcgacgaagagcaggacaatgaactgagcaccaagcagcagctgcaaaagaatgcccagcaactgcaggacaagtatgtgtgcgaactgcagttagaacggctgaccaacatcaacaaggtcagaggcgagaagaacagatttaagacagaggactttgtgaaagaagtgaaacagctgtgcgaaacccagagacagtaccacaacatcgacgaccaattcatccagcagtacatcgacctggtgtctacaagacgggagtacttcgagggccccggcaacggctctccatacggctgggacggcgacctgctgaagtggtacgagaagctgatgggcagatgcacctatttccccgaagaactgaggtccgtgaagtacgcctacagcgccgacctcttcaacgccctgaacgacctgaacaacctcgttgtgaccagggatgacaatccaaagcttgagtactacgagaagtaccacattattgagaacgtgttcaagcaaaagaagaatcccacactcaaacaaatcgccaaagagatcggcgtgcaagattacgacatccggggctatagaatcacaaagagcggcaaacctcagttcacctcttttaagctgtatcacgacctgaagaacatcttcgagcaggccaaatacctggaagatgtggaaatgctggacgagatcgccaagatcctgaccatctaccaggatgagattagcatcaagaaagccctggaccagctgcccgaactgctgacagagagcgagaaatctcagatcgcacagctcaccggctatacaggcacccacagactgagcctgaagtgcatccacattgtgatcgacgagctgtgggagagccccgagaaccagatggaaatctttaccagactgaatctgaaacctaagaaggtggaaatgagcgagatcgacagcatacccaccaccctggtcgacgagttcatcctctcacctgtggtgaagcgggccttcatccagagcatcaaggtaatcaacgcagtgatcaatcggttcggcctgccagaggacatcatcatcgagctggccagagaaaagaatagcaaggatcggagaaagttcattaacaagctgcagaaacaaaatgaggccacaagaaagaaaatcgaacagctgctggccaagtacggcaacaccaatgccaagtacatgatcgagaagatcaagctgcacgacatgcaggagggcaagtgcctgtacagcctggaggctattcctctggaagacctgctgagcaacccgacacactacgaagttgaccacattatccccagatctgtgagctttgacaacagcctgaacaacaaagtgctggtgaaacaaagcgaaaacagcaagaagggcaatcgcaccccttaccagtacctgagcagcaacgagtctaagattagctacaaccagtttaagcagcacatcctgaacctgagcaaggccaaggacagaatcagcaagaaaaaaagagatatgctgctggaagagagagatatcaacaagttcgaagtgcagaaggaattcattaaccggaacctggtggatacacggtacgccaccagagaactgtctaacctgctgaagacctacttcagcacccatgactacgccgtgaaggtgaagaccatcaacggcggcttcactaaccacctgaggaaggtgtgggatttcaagaagcacagaaaccacggctacaagcaccacgccgaagatgccctggtgatcgccaacgccgacttcctgtttaagacacataaggccctgcggagaaccgataagatcctggaacaacctggcctggaagtgaatgatacaaccgtgaaagtggacaccgaggaaaaataccaggagctgttcgagacacctaagcaagtgaagaacatcaagcagttccgggacttcaagtacagccaccgagtggacaagaagcctaaccggcagcttatcaacgacacactgtactccaccagagagattgatggcgaaacctacgtggtgcagacccttaaggatctgtacgccaaggacaacgagaaagtgaagaagctgttcaccgaaagacctcagaagatcctgatgtaccagcacgaccctaagaccttcgagaaactgatgacaatcctgaaccagtacgctgaggccaagaaccctctggctgcttattacgaggacaaaggcgagtacgtgaccaagtacgccaagaaaggcaatggacctgccatccacaagatcaagtatatcgataagaagcttggatcttacctggatgttagcaacaagtatcctgagacacagaacaagcttgtgaagctgtccctgaagagctttagattcgacatctacaagtgtgaacagggctacaagatggtgtccatcggatacctggacgtgctgaagaaagataactactactacatccctaaggacaagtacgaggccgagaagcagaaaaagaagatcaaggaatctgatctttttgtgggcagcttctactacaacgacctcatcatgtacgaggatgaactgttcagagtgataggagtgaacagcgacatcaacaatctggttgagctaaacatggtcgacattacctacaaggacttctgcgaggtgaacaacgtgacaggcgagaaaagaatcaaaaagactatcggcaagcgcgtggtcctgatcgagaagtacaccacagatattctaggcaacctgtacaagactcccctgcctaagaagccccagcttatcttcaagcggggagaactg

SEQ ID NO: 24 - DNA, 3165 bp, Artificial Sequence
  humanized ShaCas9 gene sequence
atgaagacagattacatcctgggcctggatatcggcatcaccagcgtgggctatggcatcatcaactacaatgacaagagcatcatcgacgccggagtgcggctgttccccgaggccaatgttgaaaacaacgagggcagaagaagcaagagaggcgccagaaggctgaagagacggcggattcaccggctggaaagagtgaagcagcttctgctggactacaagctgctggacagcatcgacgtgatccctcagtctaccaacccctacgagatccgggtgcggggcctgagagagaagctgaccaaagacgagctggtgatcgcgctgctccatttggctaagcgccgtggaattcacaacatcgacgtgatcgaccaggaggaggatgccagcaacgagctgagtacaaaggagcagctgagcaagaacaatctgctgctgagagataagagcatctgtgaagtcctgctggaacggtacaacgagggcaaggtgcgcggagagaaaaacagattcaaaacatctgatatcgtgaacgagatcagacagattctggagacacagaaggaagtgcaccacctggacgatagcttcatcgacaagtacatcgaactggtggaaacccgaagagaatattacgagggccctggcgaaggaagtccttacggctggggagccgatctgaagaagtggtacgagcacctgatgggccggtgcacctacttccctgaggaactgagatccgtgaagtacgcctactcagccgacctgttcaatgccctgaacgacctgaacaacctggttatccagagagacggcctgaacaagctggaatatcacgagaagtaccacatcattgaaaacgtgttcaagcagaaaaagaaacctacactgaagcagatcagcaacgagatcggcgtgaaccctgaagacatcaagggctaccggatcaccaaatctggcaaggagaatttcaccgagttcaagttgtaccacgatctcaagaagatcctgaaagaccagagcatcctagaaaatatccagctgctggaccagatcgccgagatcatcacaatctaccaggacaaggagagcattaaaaaagaactggaacagctcaccgagcctataaatgaaatcgacaaggaatctatttctgacctggctggatacaacggcacccacagactttctctgaagtgcatcaacctggtgctggaagaactgtggcacaccagcagaaatcaaatggaaatcttcgcctacctgaacatcaaacccagaaagatcgacctgcagaaagctaataaaatccccaaggacatgatcgacgaattcatcctgagccctgtggtgaagcgaacattcggccaggccatcaacgtgattaacaaggtgattgagaagtacggcgttcctaaggacatcatcatcgagctcgccagagagaacaacaccaaggacaagcagaaattcattaacgaactccagaagaaaaacgagaaaaccagacagcggatcaatgagatcatcggaaagaccggcaaccagaacgccaagagactggtggaaaagatcagactgcacgacgagcaggagggaaaatgcctgtacagcctggagagcatcccactggaggacctcctcaacaaccccaaccactacgaggtggaccacatcatacctagaagcgtgtcttttgataacagttatcaaaacaaggtgctcgtcaagcaaacagaaaacagcaagaaggggaatagaacaccttaccagtacctgaacagcggcgaggctaagctgagctacaaccagttcaagcaacacgtgctgaacctgagcaagagcaaggatagaatttccaagaagaagaaagagtacctcctggaggaacgggacattaacaagttcgaggtgcagaaggaattcatcaacagaaacctggtcgacacccgatatgccacccgcgagctgaccaattacctgaaggcctatttcagcgctaacgatatggacgtgaaggtcaagaccatcaatggaagctttaccaaccacctgcggaaggtttgggacttcaacaaggaaagaaaccacggctacaaacaccacgccgaggatgccctgatcatcgccaatgccgacttcctgtttaaggagaacaagaagctgaaggccgccaacaaagtgctggaacagcctgagagaaaggaaattgaaacgaagatcgaggtgaattccgatgagaattaccaagatctctttgtgatcccccagcaggtgaaagaaatcaaggagtttagagattttaagtacagccacagagtggacaaaaaacctaatagacagctgatcaacgatacactgtactccacaagagtgaaggacgacgacctgtacatcatatctcctatcaagaacatctacagcaaagacaacacagatctgaagaagcacttcaacaaaaacccagagaagttcctgatgtaccagaaagaccccaagaccttcgagaaactggaagttattatgaagcagtacgccaacgagaagaatcctctggccaaataccacgaggaaacaggcgagtacctgacgaagtacagcaagaccaacaacgggccaatcgtgaaaagcattaagatgttcggcaagaaagtgggcaatcacctggatgtgaccaaccagtacaacaatagccggaacaagctggtgaagctgagcttcaaaagctacagattcgacgtgtacctgacagacaagggctacaagttcgtgtccatcacctacctggacgtgctgaagaaagagaattactactacatcagcgaggccaagtacgacaaactgaaactaaacaagggcatcgatgacaaggccaagttcatcggcagcttctactacaacgacctgatcgagctggacggcgaggtgtacaccgtgatcggcgtgaataatgacaagaacaacgtcatcgagctcaatctccccgaaattagatacaaggaatactgcgagattaacaacatcaagggctccggcagactccggatcaccatcggcaagaaggtgaactccattcggaagctgtccaccgacgtgctcggcaaccggtactaccagtctttcgccaaaaagcctcagctcgtgttcaagaagggaata

SEQ ID NO: 25 - DNA, 3162 bp, Artificial Sequence
  humanized SlugCas9 gene sequence
atgaaccaaaaattcatactgggactggacatcggaatcaccagcgtgggctacggcctgatcgactacgagacaaagaatatcatcgatgccggcgttagactgttccccgaggccaacgtggaaaacaacgagggaagaaggtccaaacgtggaagcagaagactgaagcgacgccgcattcacagacttgaacgggtgaagaagctgctcgaggattataatctgctggatcagtcccagattcctcagtctacaaacccctacgccatccgcgtgaagggcctgtctgaagccctgagcaaggacgaactcgtgattgccctgctccatatcgccaagagaagaggcatccacaagatcgacgtgatcgacagcaacgacgacgtggggaacgagctcagcaccaaggaacagctgaataagaacagcaagctgctgaaagacaaatttgtgtgccagatccagctggaaagaatgaatgagggccaggtgcggggagagaaaaaccggttcaagaccgctgatatcatcaaggaaatcatccagctgctgaatgtgcagaagaacttccaccagctggacgagaacttcatcaacaagtacatcgaactggttgagatgagacgggaatacttcgagggccccggcaagggcagtccatatggctgggaaggcgaccctaaggcttggtacgagacactgatgggccactgcacctacttcccagatgagctgagaagcgtgaaatacgcctacagtgccgacctgttcaacgctctgaacgacctgaacaacctggtcatccaaagagatggactgtctaagctcgagtatcatgagaagtatcacatcatcgagaacgtgttcaagcagaagaagaaacctacactgaagcagatcgccaatgagatcaatgtcaaccctgaagatatcaagggctacagaatcacaaagtctggcaagccccagtttaccgagtttaagctctaccacgacctgaaaagcgtgctgtttgaccagagcatcctggagaacgaagacgtgctggaccagatcgctgagatcctgaccatctaccaggacaaggatagcatcaaatctaagctgacggaactggacatcctgctgaacgaggaagataaggaaaacatcgcccagctgactggctacaccgggacccaccggctcagcctgaaatgcatccggctggtcctggaagagcagtggtattctagccggaatcagatggaaatcttcacacacctgaacattaagcctaagaagatcaacctgacagccgccaacaagatcccgaaggctatgatcgacgagttcatcctgagccctgtggtgaagaggaccttcggccaggccattaaccttattaacaagatcatagaaaagtacggcgtgcctgaagatatcatcatcgagctggccagagaaaataatagcaaggacaagcagaagttcatcaatgagatgcagaaaaagaacgagaacaccagaaagagaattaacgaaatcatcggcaagtatggcaaccagaacgccaagagactggtcgagaagattagactgcacgacgagcaggagggcaagtgcctgtactcactggaaagcatccctctggaggacctgctgaacaaccccaaccactacgaggtggaccacatcattccaagatctgtgtccttcgacaactcttaccacaacaaagtgctcgtgaagcagagcgagaactccaaaaaatccaacctgaccccttaccagtactttaacagcggcaagtccaagctctcttacaaccagtttaaacaacacatcctgaacctgagcaagtcccaggatagaatcagcaaaaaaaagaaagagtatctgctggaagaacgggacatcaacaagttcgaggtgcaaaaagagttcatcaatagaaacctggtggatacccggtacgccacaagagagctgacaaactacctgaaggcctacttcagcgccaacaatatgaacgtgaaggtgaaaacgatcaacggcagcttcaccgattacctgcggaaagtgtggaagtttaagaaggaacggaaccacggctacaagcaccacgccgaggacgccctgattatcgctaatgccgatttcctgttcaaagagaacaagaagctgaaagccgtgaactctgtgctggaaaaacctgagatcgagagcaagcagctggatatccaggtggatagcgaggataactacagcgaaatgttcatcatccctaagcaggtccaggacatcaaggacttcagaaacttcaagtacagccacagagtggacaagaagcctaacagacagctgatcaacgatacactgtacagcacccggaagaaggacaactccacctacatcgtgcagaccatcaaagatatctatgccaaagataataccaccctgaagaagcagtttgacaagtcacccgagaagttcctcatgtaccaacacgatccgcggaccttcgagaagttggaagtgatcatgaagcagtacgctaatgagaagaatcctctggccaagtaccacgaggaaacaggcgagtacctgaccaaatacagcaaaaaaaacaacggccctatcgtgaaaagcctgaagtacattggaaacaagctgggcagccacctagatgtgacccaccagttcaagagcagcaccaagaagttggtgaagctgagcatcaagccttatagattcgacgtctacctgaccgacaagggatataagttcatcaccatcagctacctggacgtgctgaagaaagacaattactactacatacccgaacagaagtacgacaagctcaaactgggcaaggccatcgacaaaaacgccaagtttatcgctagcttctacaagaatgatctgatcaagctggacggcgagatctacaagatcatcggcgtgaatagcgacaccagaaacatgatcgaactggatctgcctgacatcagatacaaagaatactgcgagctgaacaatatcaagggcgaacctagaatcaaaaagaccatcggcaaaaaggtgaatagcatcgaaaaactgacaaccgacgtgctgggcaacgtgttcaccaacacccagtacacaaaacctcagctgctgttcaagcgaggaaat

SEQ ID NO: 26 - DNA, 3162 bp, Artificial Sequence
  humanized SlutCas9 gene sequence
atgagaaacagctacatcctgggcctggacatcggaatcaccagcgtgggatatggcatcatcgacagagtcaccagagaggtgatcgacgccggcgtgcggcttttccccgaggccaacgtggagaacaacgagggcagacggagcaagagaggagcccggcggctgaaaagaaggcggatccaccggctgaatagaatcaagcagctgctgaaaaacgccggcctgctggagggagatgtgctgcctaagtctaccaacccctatgagatcagagtgcggggcctccgaagccctctgaccaaagatgaactggtgatcgccctgctccacatcgccaaaagaagaggcatccacaacatcaacatcgtgggagatgacgaagaaacggacagcacactgagtaccacagcccagctgaagaagaacgagaaggctctcaagggacagtttgtttgtgaactgcaactggacagactggctaatgcccaccaagtgcggggcgagaaaaatcgatttaagacagaggacattgtgaaggaagtcagagccctgcttcagcaacagcagaacttccacaacatcgataattctttcgtggaacagtacattgccctgctggagagccggaggacctactacgagggccctggcgaaggctctccttacggctgggacggcgacattaagaagtggtacgagatgctgatgggctattgcacctacttccctgaagagctgagaagcgtgaagtacgcctacaccgccgatctgttcaacgccctgaatgacctgaacaacctggtgatcacccgggacgacaacagcaaattgacatacgccgagaagtaccatatcatcgagaacgtgttcaaacagaagaaagtacctacactgaagcagatcgccaaggaaatcggagtgaacgagagcgatattaagggctacagaatcaacaaatctgagaaacctctgttcaccagcttcaaactgtaccacgatctgaagagcgtgttcagcgaccctacaaaactggaagatatcgacttgctggaccgaatcgccgtggtgctgaccatgtaccaagatgccgaatccatgaaagccgccctgaacaccttccctgaagtgttcagcgaagcagagaaagagaagctgagcgccctcacaggctacgctggcacccatagactgtctctgaagtgcatgaacctgctgatccctgatctgtggcagacaagcctgaaccagatggaactgttcgtgaagctgaatctgaaaccacagaagctggacctgagccagtgccaccagattcctacccagctggtggacgagttcatcctgtctcctgtggtgaaaagagccttcacccaaagcatcaaggtgatcaacgccatcatccagaaatacggcctgcccgacgacatcataatcgagctggccagggaaaaaaacagcgccgataagcggaagttcctgaatcagctgcagaagaagaacgagaaggcccggcacgagatcaataccctggtggcccagtacggccagccaaatgctaagcggctggtggaaaagatcacactgcaccagcagcaggagggcaagtgtctgtactccctgaaggatatccccctggagcagctgctgaagcagccctacctgtacgaggtggaccacatcatccccagaagcgtttctttcgacaacagcatgcagaacaaggtgctggtcctggccgaagaaaacgccaaaaagggcaaccagaccccttaccagtacctgaatagcagagaggccagcatgacctaccccgaattcaaacagcacatcctgaatctgagcaaggccaaggaccggatcagcaagaagaagcggaactacctgctcgaggaaagagatatcaacaagttcgacgtgcaaaaggacttcatcaacagaaacctggttgacaccagatacgccaccagagagctcgcctctctgctgaaggcttatttcaagacacatgagctccctgtgaaagtgaagaccatcaacggcggatttacccactacctgagaaaggtgtggaagtttgacaaagatagaaacaagggctacaagcaccacgccgaggatgcactgatcatcgccaacgccgactttctgtttaagaaccagactctgaacaaaatcgaggctatcctgaacgagcccggcagagaggtggaatctgacacagtgaaggtgcagagcgaggacaattaccaggacctgtttgagaacaccaagaaggctttcgccatcaagaatttcaaggatttcaagtttagccacagagtggaccagaagcctaaccggcagctcgtgaacgacaccctgtacagcaccagagaggtgaacgaagatctgtacgtggtgcagaccctgaaggacatctacagcaaggacaacaaagacgtgaagcggctcttcgacaagcaacccgagaagttcctgatgttccagcacgaccccgagacattcaagaaattcgagctggctatgaagcaatatgccgaagagaagaaccctctggctagatactacgaggaacagggctacatcaccaagtacgccaagaaaggagacggcccacctgtgaaaagcctcaaatacatcggcaagaaggtgggaaaacacctggatgtgaccggcgactacgaggatagcaaccggaagctggtgaagctgagcctgaagtcctttagattcgatatctaccacacagacaagggctacaagatggttccaatcacttacctggatgtgcagaaaaaagagaagtactactacatccccaccgagaaatacgaagccctgaagcaggagaagggcatcaaccagaatgctcagttcattggcagcttctactacaacgacctgatcgagttcgatggcgagctgtatagagtgatcggcatcaacaacggcgacaagaatctcgttgaactcgacatggtggacattagatataaggaatactgcgagctgaactccatcaccaccacacctagaatcgttaagaccatcagccccaagacccagagcatcgagaagtacacaacagatatcctgggcaacctgtacaaagcccagcctggcaagaagcctcagttcatcttcaacaaagacgaggat

SEQ ID NO: 27 - DNA, 3168 bp, Artificial Sequence
  humanized Sa-SauriCas9 gene sequence
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaacgccgccaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccgccgccctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagagcttccgcttcgacatctacaagtgcgagcagggctacaagatggtgagcatcggctacctggacgtgctgaagaaggacaactactactacatccccaaggacaagtacgaggccgagaagcagaagaagaagataaaggagagcgacctgttcgtgggcagcttctactacaacgacctgataatgtacgaggacgagctgttccgcgtgataggcgtgaacagcgacatcaacaacctggtggagctgaacatggtggacatcacctacaaggacttctgcgaggtgaacaacgtgaccggcgagaagcgcatcaagaagaccatcggcaagcgcgtggtgctgatagagaagtacaccaccgacatcctgggcaacctgtacaagacccccctgcccaagaagccccagctgatattcaagcgcggcgagctg

SEQ ID NO: 28 - DNA, 3165 bp, Artificial Sequence
  humanized Sa-SepCas9 gene sequence
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaacgccgccaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccgccgccctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagaactaccgcttcgacgtgtacctgaccgagaagggctacaagttcgtgaccatcgcctacctgaacgtgttcaagaaggacaactactactacatccccaaggacatgtaccaggagctgaaggccaagaagaagataaaggacaccgaccagttcatcgccagcttctacaagaacgacctgataaagctgaacggcgacctgtacaagataatcggcgtgaacagcgacgaccgcaacatcatcgagctggactactacgacatcaagtacaaggactactgcgagataaacaacatcaagggcgagccccgcatcaagaagaccatcggcaagaagaccgagagcatcgagaagctgaccaccgacgtgctgggcaacctgtacctgcacagcaccgagaaggccccccagctgatattcaagcgcggcctg

SEQ ID NO: 29 - DNA, 3159 bp, Artificial Sequence
  humanized Sa-SeqCas9 gene sequence
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaacgccgccaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccgccgccctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctaccgcttcgacgtgtacctggacaacggcgtgtacaagttcgtgaccgtgaagaacctgaacgtgataaagaaggagaactactacgaggtgaacagcaagtgctacgagaaggccaagaagctgaagaagataagcgaccaggccgagttcatcgccagcttctacaacaacgacctgataaagatagacggcgagctgtaccgcgtgataggcgtgaacaccgacctgataaaccgcatcgaggtgaacatggtggacatcacctaccgcgagtacctggagaacatgaacgacaagcgcagcccccgcatcttcaagaccatcgccagcaagacccagagcatcaagaagtacagcaccgacatcctgggcaccctgtacgaggtgaacagcaagaagcacccccagatgataatgaagggc

SEQ ID NO: 30 - DNA, 3165 bp, Artificial Sequence
  humanized Sa-ShaCas9 gene sequence
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaacgccgccaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccgccgccctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagagctaccgcttcgacgtgtacctgaccgacaagggctacaagttcgtgagcatcacctacctggacgtgctgaagaaggagaactactactacatcagcgaggccaagtacgacaagctgaagctgaacaagggcatcgacgacaaggccaagttcatcggcagcttctactacaacgacctgatagagctggacggcgaggtgtacaccgtgataggcgtgaacaacgacaagaacaacgtgatagagctgaacctgcccgagatacgctacaaggagtactgcgagataaacaacatcaagggcagcggcaggctgcgcatcaccatcggcaagaaggtgaacagcatccgcaagctgagcaccgacgtgctgggcaaccgctactaccagagcttcgccaagaagccccagctggtgttcaagaagggcatc

SEQ ID NO: 31 - DNA, 3165 bp, Artificial Sequence
  humanized Sa-SlugCas9 gene sequence
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaacgccgccaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccgccgccctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctaccgcttcgacgtgtacctgaccgacaagggctacaagttcatcaccatcagctacctggacgtgctgaagaaggacaactactactacatccccgagcagaagtacgacaagctgaagctgggcaaggccatcgacaagaacgccaagttcatcgccagcttctacaagaacgacctgataaagctggacggcgagatatacaagataatcggcgtgaacagcgacacccgcaacatgatagagctggacctgcccgacatccgctacaaggagtactgcgagctgaacaacatcaagggcgagccccgcatcaagaagaccatcggcaagaaggtgaacagcatcgagaagctgaccaccgacgtgctgggcaacgtgttcaccaacacccagtacaccaagccccagctgctgttcaagcgcggcaac

SEQ ID NO: 32 - DNA, 3168 bp, Artificial Sequence
  humanized Sa-SlutCas9 gene sequence
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaacgccgccaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccgccgccctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagagcttccgcttcgacatctaccacaccgacaagggctacaagatggtgcccatcacctacctggacgtgcagaagaaggagaagtactactacatccccaccgagaagtacgaggccctgaagcaggagaagggcatcaaccagaacgcccagttcatcggcagcttctactacaacgacctgatagagttcgacggcgagctgtaccgcgtgataggcatcaacaacggcgacaagaacctggtggagctggacatggtggacatccgctacaaggagtactgcgagctgaacagcatcaccaccaccccccgcatcgtgaagaccatcagccccaagacccagagcatcgagaagtacaccaccgacatcctgggcaacctgtacaaggcccagcccggcaagaagccccagttcatcttcaacaaggacgaggac

SEQ ID NO: 33 - DNA, 21 bp, Artificial Sequence
  crRNA
agatgcgggtgatgatgctct

SEQ ID NO: 34 - DNA, 21 bp, Artificial Sequence
  crRNA
atagggttaggggccccaggc

SEQ ID NO: 35 - DNA, 21 bp, Artificial Sequence
  crRNA
ggcgcagtttactgcacaggt

SEQ ID NO: 36 - DNA, 21 bp, Artificial Sequence
  crRNA
gggaagagtgaggggaacaaa

SEQ ID NO: 37 - DNA, 20 bp, Artificial Sequence
  crRNA
gagctggtggacctagtaca

SEQ ID NO: 38 - DNA, 21 bp, Artificial Sequence
  crRNA
agtgaggggaacaaagtggac

SEQ ID NO: 39 - DNA, 21 bp, Artificial Sequence
  crRNA
gcgagcctgaggcgaacaatg

SEQ ID NO: 40 - DNA, 25 bp, Artificial Sequence
  nucleotide sequences of target site
agatgcgggtgatgatgctctttgg

SEQ ID NO: 41 - DNA, 25 bp, Artificial Sequence
  nucleotide sequences of target site
atagggttaggggccccaggccggg

SEQ ID NO: 42 - DNA, 25 bp, Artificial Sequence
  nucleotide sequences of target site
ggcgcagtttactgcacaggtgcgg

SEQ ID NO: 43 - DNA, 25 bp, Artificial Sequence
  nucleotide sequences of target site
gggaagagtgaggggaacaaagtgg

SEQ ID NO: 44 - DNA, 25 bp, Artificial Sequence
  nucleotide sequences of target site
gagctggtggacctagtacaatgga

SEQ ID NO: 45 - DNA, 26 bp, Artificial Sequence
  nucleotide sequences of target site
agtgaggggaacaaagtggacatggc

SEQ ID NO: 46 - DNA, 26 bp, Artificial Sequence
  nucleotide sequences of target site
gcgagcctgaggcgaacaatggcgga

SEQ ID NO: 47 - DNA, 20 bp, Artificial Sequence
  primer sequence for PCR amplification
cagggagtcgacgagttgaa

SEQ ID NO: 48 - DNA, 20 bp, Artificial Sequence
  primer sequence for PCR amplification
acgcagtgggtcataggctc

SEQ ID NO: 49 - DNA, 20 bp, Artificial sequence
  primer sequence for PCR amplification
agggaagaggaaatgctggg

SEQ ID NO: 50 - DNA, 20 bp, Artificial sequence
  primer sequence for PCR amplification
tccttgaacagcctgcaaac

SEQ ID NO: 51 - DNA, 19 bp, Artificial Sequence
  primer sequence for PCR amplification
atcaacccggagcagattc

SEQ ID NO: 52 - DNA, 20 bp, Artificial Sequence
  primer sequence for PCR amplification
gctgctttcctgctgtcttc

SEQ ID NO: 53 - DNA, 21 bp, Artificial Sequence
  primer sequence for PCR amplification
taattgctggcctatccacgc

SEQ ID NO: 54 - DNA, 20 bp, Artificial Sequence
  primer sequence for PCR amplification
ggactcaggcccttcctcct

SEQ ID NO: 55 - DNA, 19 bp, Artificial Sequence
  primer sequence for PCR amplification
tgagccgccagtgtacaga

SEQ ID NO: 56 - DNA, 20 bp, Artificial Sequence
  primer sequence for PCR amplification
aaggaggtctctgtctgtgc

SEQ ID NO: 57 - DNA, 21 bp, Artificial Sequence
  primer sequence for PCR amplification
cctcattgtccagaaagacca

SEQ ID NO: 58 - protein, 1054 aa, Artificial sequence
  amino acid sequence of SlugCas9-HF protein
MNQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELASVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRAQMEIFAHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATAELTNYLKAYFSANNMNVKVKTINGSFTDYLRKVWKFKKERNHGYKHHAEDALIIANADFLFKENKKLKAVNSVLEKPEIESKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 59 - DNA, 25 bp, Artificial Sequence
  Synthetic oligonucleotide
caccagagtaggctggtagatggag

SEQ ID NO: 60 - DNA, 25 bp, Artificial Sequence
  Synthetic oligonucleotide
aaacctccatctaccagcctactct

SEQ ID NO: 61 - DNA, 25 bp, Artificial Sequence
  Synthetic oligonucleotide
caccgtcagacatgagatcacagat

SEQ ID NO: 62 - DNA, 25 bp, Artificial Sequence
  Synthetic oligonucleotide
aaacatctgtgatctcatgtctgac

SEQ ID NO: 63 - DNA, 25 bp, Artificial Sequence
  nucleotide sequence of  target site for SlugCas9-HF
  misc_feature (22)..(25): n=a or t or c or g
agagtaggctggtagatggagnnnn

SEQ ID NO: 64 - DNA, 25 bp, Artificial Sequence
  nucleotide sequence of  target site for SlugCas9-HF
  misc_feature (22)..(25): n=a or t or c or g
atctgtgatctcatgtctgacnnnn

SEQ ID NO: 65 - DNA, 55 bp, Artificial Sequence
  primer sequence for PCR amplification
  misc_feature (34)..(37): n=a or t or c or g
acactctttccctacacgacgctcttccgatctnnnntgtcaggcagcagagctc

SEQ ID NO: 66 - DNA, 53 bp, Artificial Sequence
  primer sequence for PCR amplification
  misc_feature (32)..(35): n=a or t or c or g
actggagttcagacgtgtgctcttccgatctnnnnggcgatggcttcctggtc

SEQ ID NO: 67 - DNA, 52 bp, Artificial Sequence
  primer sequence for PCR amplification
caagcagaagacggcatacgagatattggcgtgactggagttcagacgtgtg

SEQ ID NO: 68 - DNA, 25 bp, Artificial Sequence
  on-target sgRNA sequence
caccggctcggagatcatcattgcg

SEQ ID NO: 69 - DNA, 25 bp, Artificial Sequence
  mismatch sgRNA sequence
caccaactcggagatcatcattgcg

SEQ ID NO: 70 - DNA, 25 bp, Artificial Sequence
  mismatch sgRNA sequence
caccgattcggagatcatcattgcg

SEQ ID NO: 71 - DNA, 25 bp, Artificial Sequence
  mismatch sgRNA sequence
caccggtccggagatcatcattgcg

SEQ ID NO: 72 - DNA, 25 bp, Artificial Sequence
  mismatch sgRNA sequence
caccggcctggagatcatcattgcg

SEQ ID NO: 73 - DNA, 25 bp, Artificial Sequence
  mismatch sgRNA sequence
caccggcttagagatcatcattgcg

SEQ ID NO: 74 - DNA, 25 bp, Artificial Sequence
  mismatch sgRNA sequence
caccggctcaaagatcatcattgcg

SEQ ID NO: 75 - DNA, 25 bp, Artificial Sequence
  mismatch sgRNA sequence
caccggctcgaggatcatcattgcg

SEQ ID NO: 76 - DNA, 25 bp, Artificial Sequence
  mismatch sgRNA sequence
caccggctcgggaatcatcattgcg

SEQ ID NO: 77 - DNA, 25 bp, Artificial Sequence
  mismatch sgRNA sequence
caccggctcggaagtcatcattgcg

SEQ ID NO: 78 - DNA, 25 bp, Artificial Sequence
  mismatch sgRNA sequence
caccggctcggaggccatcattgcg

SEQ ID NO: 79 - DNA, 25 bp, Artificial Sequence
  mismatch sgRNA sequence
caccggctcggagactatcattgcg

SEQ ID NO: 80 - DNA, 25 bp, Artificial Sequence
  mismatch sgRNA sequence
caccggctcggagattgtcattgcg

SEQ ID NO: 81 - DNA, 25 bp, Artificial Sequence
  mismatch sgRNA sequence
caccggctcggagatcgccattgcg

SEQ ID NO: 82 - DNA, 25 bp, Artificial Sequence
  mismatch sgRNA sequence
caccggctcggagatcactattgcg

SEQ ID NO: 83 - DNA, 25 bp, Artificial Sequence
  mismatch sgRNA sequence
caccggctcggagatcattgttgcg

SEQ ID NO: 84 - DNA, 25 bp, Artificial Sequence
  mismatch sgRNA sequence
caccggctcggagatcatcgctgcg

SEQ ID NO: 85 - DNA, 25 bp, Artificial Sequence
  mismatch sgRNA sequence
caccggctcggagatcatcaccgcg

SEQ ID NO: 86 - DNA, 25 bp, Artificial Sequence
  mismatch sgRNA sequence
caccggctcggagatcatcatcacg

SEQ ID NO: 87 - DNA, 25 bp, Artificial Sequence
  mismatch sgRNA sequence
caccggctcggagatcatcattatg

SEQ ID NO: 88 - DNA, 25 bp, Artificial Sequence
  mismatch sgRNA sequence
caccggctcggagatcatcattgta

SEQ ID NO: 89 - DNA, 25 bp, Artificial Sequence
  on-target sgRNA sequence
aaaccgcaatgatgatctccgagcc

SEQ ID NO: 90 - DNA, 25 bp, Artificial Sequence
  mismatch sgRNA sequence
aaaccgcaatgatgatctccgagtt

SEQ ID NO: 91 - DNA, 25 bp, Artificial Sequence
  mismatch sgRNA sequence
aaaccgcaatgatgatctccgaatc

SEQ ID NO: 92 - DNA, 25 bp, Artificial Sequence
  mismatch sgRNA sequence
aaaccgcaatgatgatctccggacc

SEQ ID NO: 93 - DNA, 25 bp, Artificial Sequence
  mismatch sgRNA sequence
aaaccgcaatgatgatctccaggcc

SEQ ID NO: 94 - DNA, 25 bp, Artificial Sequence
  mismatch sgRNA sequence
aaaccgcaatgatgatctctaagcc

SEQ ID NO: 95 - DNA, 25 bp, Artificial Sequence
  mismatch sgRNA sequence
aaaccgcaatgatgatctttgagcc

SEQ ID NO: 96 - DNA, 25 bp, Artificial Sequence
  mismatch sgRNA sequence
aaaccgcaatgatgatcctcgagcc

SEQ ID NO: 97 - DNA, 25 bp, Artificial Sequence
  mismatch sgRNA sequence
aaaccgcaatgatgattcccgagcc

SEQ ID NO: 98 - DNA, 25 bp, Artificial Sequence
  mismatch sgRNA sequence
aaaccgcaatgatgacttccgagcc

SEQ ID NO: 99 - DNA, 25 bp, Artificial Sequence
  mismatch sgRNA sequence
aaaccgcaatgatggcctccgagcc

SEQ ID NO: 100 - DNA, 25 bp, Artificial Sequence
  mismatch sgRNA sequence
aaaccgcaatgatagtctccgagcc

SEQ ID NO: 101 - DNA, 25 bp, Artificial Sequence
  mismatch sgRNA sequence
aaaccgcaatgacaatctccgagcc

SEQ ID NO: 102 - DNA, 25 bp, Artificial Sequence
  mismatch sgRNA sequence
aaaccgcaatggcgatctccgagcc

SEQ ID NO: 103 - DNA, 25 bp, Artificial Sequence
  mismatch sgRNA sequence
aaaccgcaatagtgatctccgagcc

SEQ ID NO: 104 - DNA, 25 bp, Artificial Sequence
  mismatch sgRNA sequence
aaaccgcaacaatgatctccgagcc

SEQ ID NO: 105 - DNA, 25 bp, Artificial Sequence
  mismatch sgRNA sequence
aaaccgcagcgatgatctccgagcc

SEQ ID NO: 106 - DNA, 25 bp, Artificial Sequence
  mismatch sgRNA sequence
aaaccgcggtgatgatctccgagcc

SEQ ID NO: 107 - DNA, 25 bp, Artificial Sequence
  mismatch sgRNA sequence
aaaccgtgatgatgatctccgagcc

SEQ ID NO: 108 - DNA, 25 bp, Artificial Sequence
  mismatch sgRNA sequence
aaaccataatgatgatctccgagcc

SEQ ID NO: 109 - DNA, 25 bp, Artificial Sequence
  mismatch sgRNA sequence
aaactacaatgatgatctccgagcc

SEQ ID NO: 110 - DNA, 25 bp, Artificial Sequence
  nucleotide sequence of the target site
  misc_feature (21)..(25): n=a or t or c or g
ggctcggagatcatcattgcgnnnn

SEQ ID NO: 111 - DNA, 1649 bp, Artificial Sequence
  CMV-ATG-target site-CTGG-GFP nucleotide sequence
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagtctagagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatgcatgcgagaaaagccttgtttgccaccatgggctcggagatcatcattgcgctgggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaag

SEQ ID NO: 112 - DNA, 3162 bp, Artificial Sequence
  humanized SlugCas9-HF gene sequence
atgaaccaaaaattcatactgggactggacatcggaatcaccagcgtgggctacggcctgatcgactacgagacaaagaatatcatcgatgccggcgttagactgttccccgaggccaacgtggaaaacaacgagggaagaaggtccaaacgtggaagcagaagactgaagcgacgccgcattcacagacttgaacgggtgaagaagctgctcgaggattataatctgctggatcagtcccagattcctcagtctacaaacccctacgccatccgcgtgaagggcctgtctgaagccctgagcaaggacgaactcgtgattgccctgctccatatcgccaagagaagaggcatccacaagatcgacgtgatcgacagcaacgacgacgtggggaacgagctcagcaccaaggaacagctgaataagaacagcaagctgctgaaagacaaatttgtgtgccagatccagctggaaagaatgaatgagggccaggtgcggggagagaaaaaccggttcaagaccgctgatatcatcaaggaaatcatccagctgctgaatgtgcagaagaacttccaccagctggacgagaacttcatcaacaagtacatcgaactggttgagatgagacgggaatacttcgagggccccggcaagggcagtccatatggctgggaaggcgaccctaaggcttggtacgagacactgatgggccactgcacctacttcccagatgagctggctagcgtgaaatacgcctacagtgccgacctgttcaacgctctgaacgacctgaacaacctggtcatccaaagagatggactgtctaagctcgagtatcatgagaagtatcacatcatcgagaacgtgttcaagcagaagaagaaacctacactgaagcagatcgccaatgagatcaatgtcaaccctgaagatatcaagggctacagaatcacaaagtctggcaagccccagtttaccgagtttaagctctaccacgacctgaaaagcgtgctgtttgaccagagcatcctggagaacgaagacgtgctggaccagatcgctgagatcctgaccatctaccaggacaaggatagcatcaaatctaagctgacggaactggacatcctgctgaacgaggaagataaggaaaacatcgcccagctgactggctacaccgggacccaccggctcagcctgaaatgcatccggctggtcctggaagagcagtggtattctagccgggctcagatggaaatcttcgcccacctgaacattaagcctaagaagatcaacctgacagccgccaacaagatcccgaaggctatgatcgacgagttcatcctgagccctgtggtgaagaggaccttcggccaggccattaaccttattaacaagatcatagaaaagtacggcgtgcctgaagatatcatcatcgagctggccagagaaaataatagcaaggacaagcagaagttcatcaatgagatgcagaaaaagaacgagaacaccagaaagagaattaacgaaatcatcggcaagtatggcaaccagaacgccaagagactggtcgagaagattagactgcacgacgagcaggagggcaagtgcctgtactcactggaaagcatccctctggaggacctgctgaacaaccccaaccactacgaggtggaccacatcattccaagatctgtgtccttcgacaactcttaccacaacaaagtgctcgtgaagcagagcgagaactccaaaaaatccaacctgaccccttaccagtactttaacagcggcaagtccaagctctcttacaaccagtttaaacaacacatcctgaacctgagcaagtcccaggatagaatcagcaaaaaaaagaaagagtatctgctggaagaacgggacatcaacaagttcgaggtgcaaaaagagttcatcaatagaaacctggtggatacccggtacgccacagccgagctgacaaactacctgaaggcctacttcagcgccaacaatatgaacgtgaaggtgaaaacgatcaacggcagcttcaccgattacctgcggaaagtgtggaagtttaagaaggaacggaaccacggctacaagcaccacgccgaggacgccctgattatcgctaatgccgatttcctgttcaaagagaacaagaagctgaaagccgtgaactctgtgctggaaaaacctgagatcgagagcaagcagctggatatccaggtggatagcgaggataactacagcgaaatgttcatcatccctaagcaggtccaggacatcaaggacttcagaaacttcaagtacagccacagagtggacaagaagcctaacagacagctgatcaacgatacactgtacagcacccggaagaaggacaactccacctacatcgtgcagaccatcaaagatatctatgccaaagataataccaccctgaagaagcagtttgacaagtcacccgagaagttcctcatgtaccaacacgatccgcggaccttcgagaagttggaagtgatcatgaagcagtacgctaatgagaagaatcctctggccaagtaccacgaggaaacaggcgagtacctgaccaaatacagcaaaaaaaacaacggccctatcgtgaaaagcctgaagtacattggaaacaagctgggcagccacctagatgtgacccaccagttcaagagcagcaccaagaagttggtgaagctgagcatcaagccttatagattcgacgtctacctgaccgacaagggatataagttcatcaccatcagctacctggacgtgctgaagaaagacaattactactacatacccgaacagaagtacgacaagctcaaactgggcaaggccatcgacaaaaacgccaagtttatcgctagcttctacaagaatgatctgatcaagctggacggcgagatctacaagatcatcggcgtgaatagcgacaccagaaacatgatcgaactggatctgcctgacatcagatacaaagaatactgcgagctgaacaatatcaagggcgaacctagaatcaaaaagaccatcggcaaaaaggtgaatagcatcgaaaaactgacaaccgacgtgctgggcaacgtgttcaccaacacccagtacacaaaacctcagctgctgttcaagcgaggaaat

SEQ ID NO: 113 - DNA, 1656 bp, Artificial Sequence
  CMV-ATG-target site-NNNNNNN-GFP nucleotide sequence
  misc_feature (936)..(942): n=a or t or c or g
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagtctagagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatgcatgcgagaaaagccttgtttgccaccatggaacggctcggagatcatcattgcgnnnnnnngtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaag

(19) United States
(12) Patent Application Publication
(10) Pub. No.: US 2024/0175055 A1
(43) Pub. Date: May 30, 2024

(54) CRISPR/CAS9 GENE EDITING SYSTEM AND APPLICATION THEREOF

(71) Applicant: FUDAN UNIVERSITY, Yangpu District Shanghai (CN)

(72) Inventors: Yongming WANG, Yangpu District Shanghai (CN); Ziying HU, Yangpu District Shanghai (CN); Daqi WANG, Yangpu District Shanghai (CN); Shuai WANG, Yangpu District Shanghai (CN)

(21) Appl. No.: 17/633,354

(22) PCT Filed: Aug. 7, 2020
(86) PCT No.: PCT/CN2020/107880

§ 371 Date: Feb. 7, 2022

(30) Foreign Application Priority Data
Aug. 8, 2019 (CN) 201910731390.2
Aug. 8, 2019 (CN) 201910731396.X
Aug. 8, 2019 (CN) 201910731398.9
Aug. 8, 2019 (CN) 201910731401.7
Aug. 8, 2019 (CN) 201910731402.1
Aug. 8, 2019 (CN) 201910731412.5
Aug. 8, 2019 (CN) 201910731794.1
Aug. 8, 2019 (CN) 201910731795.6
Aug. 8, 2019 (CN) 201910731802.2
Aug. 8, 2019 (CN) 201910731803.7

(51) Int. Cl. C12N 15/90; C12N 9/22; C12N 15/11; C12N 15/86

(52) U.S. Cl. CPC C12N 15/907; C12N 9/22; C12N 15/11; C12N 15/86; C12N 2310/20; C12N 2750/14143; C12N 2800/80

(57) ABSTRACT

A CRISPR/Cas9 gene editing system and application thereof. The gene editing system is a complex formed by a specific Cas9 protein and sgRNA, and can precisely position a target DNA sequence and cause cutting so that double strand breaks occur to the target DNA sequence; the gene editing refers to intracellular or in vitro gene editing. The used specific Cas9 protein is small and only has about 1000 amino acids, and thus an identified PAM sequence is simple; the Cas9 protein has an amino acid sequence represented by any one of SEQ ID NOs: 1-10 and 58; the sgRNA has a nucleotide sequence represented by SEQ ID NO: 11.

### CROSS REFERENCE TO RELATED APPLICATION

This application is the U.S. National Phase of PCT/CN2020/107880, filed on Aug. 7, 2020, which claims priority to Chinese Patent Application Ser. Nos. 201910731795.6, 201910731402.1, 201910731802.2, 201910731390.2, 201910731398.9, 201910731396.X, 201910731412.5, 201910731794.1, 201910731803.7, and 201910731401.7, all filed on Aug. 8, 2019, the entire disclosures of which are incorporated by reference herein.

### SEQUENCE LISTING

The present disclosure incorporates by reference in its entirety the material in the accompanying ASCII text file designated Sequence_Listing_ST25.txt, created Aug. 9, 2022, and having a file size of 172,496 bytes.

### TECHNICAL FIELD

The present disclosure belongs to the technical field of gene editing, and particularly relates to a CRISPR/Cas9 system capable of performing gene editing in cells; and related applications thereof.

### BACKGROUND

CRISPR/Cas9 is an acquired immune system that bacteria and archaebacteria have evolved to resist the invasion of foreign viruses or plasmids. In the CRISPR/Cas9 system, crRNA (CRISPR-derived RNA), tracrRNA (trans-activating RNA) and a Cas9 protein forms a complex which recognizes the PAM (Protospacer Adjacent Motif) sequence of the target site, crRNA forms a complementary structure with the target DNA sequence, and the Cas9 protein performs the function of DNA cleavage, causing DNA break damage. In the complex, tracrRNA and crRNA can be fused into a single guide RNA (sgRNA) through a linker sequence. When DNA is broken and damaged, there are two main repair mechanisms for DNA damage in cells which are responsible for repairing: non-homologous end-joining (NHEJ) and homologous recombination (HR). The NHEJ repair may cause the deletion or insertion of base(s), and thus can be used for gene knockout. In the case that a homologous template is provided, the HR repair can be used for site-specific insertion and precise base substitution for a gene.

In addition to basic scientific research, CRISPR/Cas9 also has a wide range of clinical applications. When the CRISPR/Cas9 system is used for gene therapy, Cas9 and sgRNA need to be introduced into a body. At present, AAV virus is the most effective delivery vector for gene therapy. However, the DNA capable of being packaged by the AAV virus generally does not exceed 4.5 kb. SpCas9 is widely used because it recognizes a simple PAM sequence (recognizing NGG) and has high activity. However, a SpCas9 protein itself has 1368 amino acids, and thus when it is in complex with sgRNA and a promoter, it cannot be effectively packaged into AAV virus, thereby limiting the clinical applications thereof. In order to overcome the above problems, researchers have invented several small Cas9 proteins, comprising SaCas9 (the PAM sequence is NNGRRT), St1Cas9 (the PAM sequence is NNAGAW), NmCas9 (the PAM sequence is NNNNGATT), Nme2Cas9 (the PAM sequence is NNNNCC), and CjCas9 (the PAM sequence is NNNNRYAC). However, these Cas9 proteins either tend to be off-target (i.e., performing cleavage at a non-target site), or recognize complicated PAM sequences, or have low editing activity, and thus are difficult to be widely used.

Therefore, a small CRISPR/Cas9 system having high editing activity and high specificity, and recognizing a simple PAM sequence is the hope for solving the above problems.

### SUMMARY

In view of the above problems, the present disclosure aims to provide a new CRISPR/Cas9 gene editing system having high editing activity, high specificity, and a small Cas9 protein, and recognizing a simple PAM sequence; and the applications thereof.

Thus, in a first aspect, the present disclosure provides a CRISPR/Cas9 system for gene editing in cells or in vitro, wherein the CRISPR/Cas9 system is a complex of a Cas9 protein and a sgRNA, which is capable of accurately locating and cleaving a target DNA sequence so as to cause double-strand break damage to the target DNA sequence, where
the Cas9 protein is:
a SauriCas9 protein having an amino acid sequence represented by SEQ ID NO: 1,
a ShaCas9 protein having an amino acid sequence represented by SEQ ID NO: 2,
a SlugCas9 protein having an amino acid sequence represented by SEQ ID NO: 3,
a SlutCas9 protein having an amino acid sequence represented by SEQ ID NO: 4,
a Sa-SauriCas9 protein having an amino acid sequence represented by SEQ ID NO: 5,
a Sa-SepCas9 protein having an amino acid sequence represented by SEQ ID NO: 6,
a Sa-SeqCas9 protein having an amino acid sequence represented by SEQ ID NO: 7,
a Sa-ShaCas9 protein having an amino acid sequence represented by SEQ ID NO: 8,
a Sa-SlugCas9 protein having an amino acid sequence represented by SEQ ID NO: 9,
a Sa-SlutCas9 protein having an amino acid sequence represented by SEQ ID NO: 10,
a SlugCas9-HF protein having an amino acid sequence represented by SEQ ID NO: 58, or
a Cas9 protein having an amino acid sequence that is at least 80% identical to the amino acid sequence represented by any one of SEQ ID NOS: 1-10 and SEQ ID NO: 58; and
the sgRNA has a nucleotide sequence represented by SEQ ID NO: 11, or is a modified sgRNA sequence based on SEQ ID NO: 11.

In a second aspect, the present disclosure provides a method for gene editing in cells with the CRISPR/Cas9 system for gene editing according to the first aspect of the present disclosure, wherein the method edits a target DNA sequence by recognizing and locating the target DNA sequence with a complex of a Cas9 protein and a sgRNA, and the method comprises the steps of:
(1) synthesizing a Cas9 gene sequence and cloning it into an expression vector such as pAAV2_ITR, to obtain an expression vector cloned with the Cas9 gene sequence, such as pAAV2_Cas9_ITR, wherein the Cas9 gene sequence:
(a) has a nucleotide sequence encoding an amino acid sequence represented by any one of SEQ ID NOs: 1-10 and SEQ ID NO: 58;
(b) has a nucleotide sequence encoding an amino acid sequence that is at least 80% identical to the amino acid sequence represented by any one of SEQ ID NOs: 1-10 and SEQ ID NO: 58; or
(c) is a humanized Cas9 gene sequence, for example, the one having a nucleotide sequence represented by any one of SEQ ID NOs: 23-32 and SEQ ID NO: 112;
(2) synthesizing oligo single-stranded DNAs corresponding to the sgRNA, i.e., an oligo forward-strand sequence and an oligo reverse-strand sequence, and annealing and ligating the oligo forward-strand sequence and the oligo reverse-strand sequence to a restriction site of the expression vector cloned with the Cas9 gene sequence, such as the BsaI digestion site of plasmid pAAV2_Cas9_U6_BsaI, to obtain an expression vector, such as pAAV2_Cas9-hU6-sgRNA, for expressing the Cas9 protein and the sgRNA; wherein the sgRNA has a nucleotide sequence represented by SEQ ID NO: 11, or a nucleotide sequence that is at least 80% identical to a nucleotide sequence represented by SEQ ID NO: 11, or a modification comprising, e.g., phosphorylation, shortening, lengthening, sulfurization, methylation or hydroxylation, based on the nucleotide sequence represented by SEQ ID NO: 11; and
(3) delivering the expression vector expressing the Cas9 protein and the sgRNA into cells comprising a target site to edit the target site.

In a third aspect, the present disclosure provides a kit of a CRISPR/Cas9 system for gene editing, the kit comprises:
(1) a Cas9 protein and a sgRNA, wherein the Cas9 protein is:
a SauriCas9 protein having an amino acid sequence represented by SEQ ID NO: 1,
a ShaCas9 protein having an amino acid sequence represented by SEQ ID NO: 2,
a SlugCas9 protein having an amino acid sequence represented by SEQ ID NO: 3,
a SlutCas9 protein having an amino acid sequence represented by SEQ ID NO: 4,
a Sa-SauriCas9 protein having an amino acid sequence represented by SEQ ID NO: 5,
a Sa-SepCas9 protein having an amino acid sequence represented by SEQ ID NO: 6,
a Sa-SeqCas9 protein having an amino acid sequence represented by SEQ ID NO: 7,
a Sa-ShaCas9 protein having an amino acid sequence represented by SEQ ID NO: 8,
a Sa-SlugCas9 protein having an amino acid sequence represented by SEQ ID NO: 9,
a Sa-SlutCas9 protein having an amino acid sequence represented by SEQ ID NO: 10,
a SlugCas9-HF protein having an amino acid sequence represented by SEQ ID NO: 58, or
a Cas9 protein having an amino acid sequence that is at least 80% identical to the amino acid sequence represented by any one of SEQ ID NOS: 1-10 and SEQ ID NO: 58; and
the sgRNA has a nucleotide sequence represented by SEQ ID NO: 11, or is a modified sgRNA sequence based on SEQ ID NO: 11; or
(2) an expression vector cloned with a Cas9 gene sequence and a sequence expressing a sgRNA, wherein
the Cas9 gene sequence:
   (a) has a nucleotide sequence encoding an amino acid sequence represented by any one of SEQ ID NO: 1-10 and SEQ ID NO: 58;
   (b) has a nucleotide sequence encoding an amino acid sequence that is at least 80% identical to the amino acid sequence represented by any one of SEQ ID NO: 1-10 and SEQ ID NO: 58; or
   (c) is a humanized Cas9 gene sequence, for example, the one having a nucleotide sequence represented by any one of SEQ ID NOs: 23-32 and SEQ ID NO: 112; and
the sgRNA has a nucleotide sequence represented by SEQ ID NO: 11, or a modified sgRNA sequence based on SEQ ID NO: 11.

In a fourth aspect, the present disclosure provides use of the CRISPR/Cas9 system for gene editing according to the first aspect of the present disclosure in gene knockout, site-directed base change, site-directed insertion, regulation of gene transcription level, regulation of DNA methylation, modification of DNA acetylation, modification of histone acetylation, single-base conversion or chromatin imaging tracking.

Compared with the existing CRISPR/Cas9 systems for gene editing in the prior art, the CRISPR/Cas9 gene editing system of the present disclosure comprises a smaller Cas9 protein with fewer amino acids than the prior art, and thus can be effectively packaged. Furthermore, the CRISPR/Cas9 gene editing system of the present disclosure can recognize a relatively simple PAM sequence, and thus can target more DNA sequences in the genome and has higher editing efficiency.

### BRIEF DESCRIPTION OF THE DRAWINGS

FIG. 1 is a schematic diagram of the cleavage of the target DNA sequence by using a CRISPR/Cas9 gene editing system, wherein the gray oval represents a Cas9 protein, the black curve represents a sgRNA sequence, and the darkened area in one chain of the genome represents a PAM sequence.

FIG. 2 is a schematic diagram of the map of plasmids pAAV2_Cas9_U6_BsaI, comprising elements such as AAV2 ITR, CMV enhancer, CMV promoter, SV40 NLS, Cas9, nucleoplasmin NLS, 3×HA, bGH poly(A), human U6 promoter (hU6), BsaI endonuclease site, and sgRNA scaffold sequence, and the like.

FIG. 3a to FIG. 3j represent some results from next-generation sequencing of the DNA sequence of the target site upon editing, wherein the edited results comprise deletions, insertions or mismatches, and the last 4 bp or 5 bp represents the PAM sequence, which is NNGG, NNGRM, NNGG, NNGR, NNGG, NNGG, NNGRM, NNGRM, NNGG and NNGRR, respectively, from FIGS. 3a to 3j.

FIG. 3k shows the editing of two target sites by the SlugCas9-HF gene editing system, wherein the X axis represents the two target sites, G4 and G7, and the Y axis represents indel efficiency.

FIG. 4a to FIG. 4j represent digestion results of T7 Endonuclease I at the endogenous site, wherein the arrows indicate the size of the cleaved fragments.

FIG. 4k shows the detection result of the specificity of SlugCas9-HF gene editing system in a GFP-reporter cell line, wherein the top shows the schematic diagram of the GFP reporter system. A specific target DNA sequence and a PAM are inserted between the start codon ATG and the GFP-coding sequence to cause a GFP frameshift mutation. When the target DNA is cleaved by the gene editing system, some cells can restore the GFP reading frame through the self-repairing system of the cells to produce green fluorescence. In the histogram of FIG. 4k, the Y-axis represents the GFP positive ratio, and the X-axis represents the sequences of on-target sgRNA and mismatch sgRNAs.

### DETAILED DESCRIPTION

The following embodiments are provided to further illustrate the present disclosure, but not to limit the protection scope of the present disclosure in any form; on the contrary, the protection scope of the present disclosure is defined by the appended claims.

As described in the Background Section, current CRISPR/Cas9 gene editing systems have various problems. For example, the Cas9 protein is too large, so that the system cannot be effectively packaged into a vector such as a virus. For another example, the current PAM sequences are relatively complicated, resulting in a small editing range, and it is difficult for the current CRISPR/Cas9 gene editing systems to be widely used. For another example, the current small Cas9 proteins generally have low editing activity.

For the above problems, the present disclosure aims to provide a new CRISPR/Cas9 gene editing system having high editing activity, high specificity, and a small Cas9 protein, and recognizing a simple PAM sequence; and the applications thereof.

Thus, in the first aspect, the present disclosure provides a CRISPR/Cas9 system for gene editing in cells or in vitro, wherein the CRISPR/Cas9 system is a complex of a Cas9 protein and a sgRNA, and is capable of accurately locating and cleaving a target DNA sequence so as to cause double-strand break damage to the target DNA sequence, where
the Cas9 protein is:
a SauriCas9 protein having an amino acid sequence represented by SEQ ID NO: 1,
a ShaCas9 protein having an amino acid sequence represented by SEQ ID NO: 2,
a SlugCas9 protein having an amino acid sequence represented by SEQ ID NO: 3,
a SlutCas9 protein having an amino acid sequence represented by SEQ ID NO: 4,
a Sa-SauriCas9 protein having an amino acid sequence represented by SEQ ID NO: 5,
a Sa-SepCas9 protein having an amino acid sequence represented by SEQ ID NO: 6,
a Sa-SeqCas9 protein having an amino acid sequence represented by SEQ ID NO: 7,
a Sa-ShaCas9 protein having an amino acid sequence represented by SEQ ID NO: 8,
a Sa-SlugCas9 protein having an amino acid sequence represented by SEQ ID NO: 9,
a Sa-SlutCas9 protein having an amino acid sequence represented by SEQ ID NO: 10,
a SlugCas9-HF protein having an amino acid sequence represented by SEQ ID NO: 58, or
the Cas9 protein having an amino acid sequence that is at least 80% identical to the amino acid sequence represented by any one of SEQ ID NOs: 1-10 and SEQ ID NO: 58; and
the sgRNA has a nucleotide sequence represented by SEQ ID NO: 11, or is a modified sgRNA sequence based on SEQ ID NO: 11.

In the context of the present disclosure, the sequences of SEQ ID NOs: 1-11 and SEQ ID NO: 58 are as follows.

the amino acid sequence of SauriCas9 protein:
SEQ ID NO: 1
MQENQQKQNYILGLDIGITSVGYGLIDSKTREVIDAGVRLFPEADSENNSNRRSKRGARRLK
RRRIHRLNRVKDLLADYQMIDLNNVPKSTDPYTIRVKGLREPLTKEEFAIALLHIAKRRGLH
NISVSMGDEEQDNELSTKQQLQKNAQQLQDKYVCELQLERLTNINKVRGEKNRFKTEDFVKE
VKQLCETQRQYHNIDDQFIQQYIDLVSTRREYFEGPGNGSPYGWDGDLLKWYEKLMGRCTYF
PEELRSVKYAYSADLFNALNDLNNLVVTRDDNPKLEYYEKYHIIENVFKQKKNPTLKQIAKE
IGVQDYDIRGYRITKSGKPQFTSFKLYHDLKNIFEQAKYLEDVEMLDEIAKILTIYQDEISI
KKALDQLPELLTESEKSQIAQLTGYTGTHRLSLKCIHIVIDELWESPENQMEIFTRLNLKPK
KVEMSEIDSIPTTLVDEFILSPVVKRAFIQSIKVINAVINRFGLPEDIIIELAREKNSKDRR
KFINKLOKQNEATRKKIEQLLAKYGNTNAKYMIEKIKLHDMQEGKCLYSLEAIPLEDLLSNP
THYEVDHHIPRSVSFDNSLNNKVLVKQSENSKKGNRTPYQYLSSNESKISYNQFKQHILNLS
KAKDRISKKKRDMLLEERDINKFEVQKEFINRNLVDTRYATRELSNLLKTYFSTHDYAVKVK
TINGGFTNHLRKVWDFKKHRNHGYKHHAEDALVIANADFLFKTHKALRRTDKILEQPGLEVN
DTTVKVDTEEKYQELFETPKQVKNIKQFRDFKYSHRVDKKPNRQLINDTLYSTREIDGETYV
VQTLKDLYAKDNEKVKKLFTERPQKILMYQHDPKTFEKLMTILNQYABAKNPLAAYYEDKGE
YVTKYAKKGNGPAIHKIKYIDKKLGSYLDVSNKYPETQNKLVKLSLKSFRFDIYKCEQGYKM
VSIGYLDVLKKDNYYYIPKDKYEAEKQKKKIKESDLFVGSFYYNDLIMYEDELFRVIGVNSD
INNLVELNMVDITYKDFCEVNNVTGEKRIKKTIGKRVVLIEKYTTDILGNLYKTPLPKKPQL
IFKRGEL;
the amino acid sequence of ShaCas9 protein:
SEQ ID NO: 2
MKTDYILGLDIGITSVGYGIINYNDKSIIDAGVRLFPEANVENNEGRRSKRGARRLKRRRIH
RLERVKQLLLDYKLLDSIDVIPQSTNPYEIRVRGLREKLTKDELVIALLHLAKRRGIHNIDV
IDQEEDASNELSTKEQLSKNNLLLRDKSICEVLLERYNEGKVRGEKNRFKTSDIVNEIRQIL
ETQKEVHHLDDSFIDKYIELVETRREYYEGPGEGSPYGWGADLKKWYEHLMGRCTYFPEELR
SVKYAYSADLFNALNDLNNLVIQRDGLNKLEYHEKYHIIENVFKQKKKPTLKQISNEIGVNP
EDIKGYRITKSGKENFTEFKLYHDLKKILKDQSILENIQLLDQIAEIITIYQDKESIKKELE
QLTEPINEIDKESISDLAGYNGTHRLSLKCINLVLEELWHTSRNQMEIFAYLNIKPRKIDLQ
KANKIPKDMIDEFILSPVVKRTFGQAINVINKVIEKYGVPKDIIIELARENNTKDKQKFINE
LQKKNEKTRORINEIIGKTGNQNAKRLVEKIRLHDEQEGKCLYSLESIPLEDLLNNPNHYEV
DHIIPRSVSFDNSYQNKVLVKQTENSKKGNRTPYQYLNSGEAKLSYNQFKQHVLNLSKSKDR
ISKKKKEYLLEERDINKFEVQKEFINRNLVDTRYATRELTNYLKAYFSANDMDVKVKTINGS
FTNHLRKVWDFNKERNHGYKHHAEDALIIANADFLFKENKKLKAANKVLEQPERKEIETKIE
VNSDENYQDLFVIPQQVKEIKEFRDFKYSHRVDKKPNRQLINDTLYSTRVKDDDLYIISPIK
NIYSKDNTDLKKHENKNPEKFLMYQKDPKTFEKLEVIMKQYANEKNPLAKYHEETGEYLTKY
SKTNNGPIVKSIKMFGKKVGNHLDVTNQYNNSRNKLVKLSFKSYRFDVYLTDKGYKFVSITY
LDVLKKENYYYISEAKYDKLKLNKGIDDKAKFIGSFYYNDLIELDGEVYTVIGVNNDKNNVI
ELNLPEIRYKEYCEINNIKGSGRLRITIGKKVNSIRKLSTDVLGNRYYQSFAKKPQLVFKKG
I*;
the amino acid sequence of SlugCas9 protein:
SEQ ID NO: 3
MNQKFILGLDIGITSVGYGLIDYETKNIIDAGVRLFPEANVENNEGRRSKRGSRRLKRRRIH
RLERVKKLLEDYNLLDQSQIPQSTNPYAIRVKGLSEALSKDELVIALLHIAKRRGIHKIDVI
DSNDDVGNELSTKEQLNKNSKLLKDKFVCQIQLERMNEGQVRGEKNRFKTADIIKEIIQLLN
VQKNFHQLDENFINKYIELVEMRREYFEGPGKGSPYGWEGDPKAWYETLMGHCTYFPDELRS
VKYAYSADLFNALNDLNNLVIQRDGLSKLEYHEKYHIIENVFKQKKKPTLKQIANEINVNPE
DIKGYRITKSGKPQFTEFKLYHDLKSVLFDQSILENEDVLDQIAEILTIYQDKDSIKSKLTE
LDILLNEEDKENIAQLTGYTGTHRLSLKCIRLVLEEQWYSSRNOMEIFTHLNIKPKKINLTA
ANKIPKAMIDEFILSPVVKRTFGQAINLINKIIEKYGVPEDIIIELARENNSKDKQKFINEM
QKKNENTRKRINEIIGKYGNQNAKRLVEKIRLHDEQEGKCLYSLESIPLEDLLNNPNHYEVD
HIIPRSVSFDNSYHNKVLVKQSENSKKSNLTPYQYENSGKSKLSYNQFKQHILNLSKSQDRI
SKKKKEYLLEERDINKFEVQKEFINRNLVDTRYATRELTNYLKAYFSANNMNVKVKTINGSF
TDYLRKVWKFKKERNHGYKHHAEDALIIANADFLFKENKKLKAVNSVLEKPEIESKQLDIQV
DSEDNYSEMFIIPKQVQDIKDFRNFKYSHRVDKKPNRQLINDTLYSTRKKDNSTYIVQTIKD
IYAKDNTTLKKQFDKSPEKFLMYQHDPRTFEKLEVIMKQYANEKNPLAKYHEETGEYLTKYS
KKNNGPIVKSLKYIGNKLGSHLDVTHQFKSSTKKLVKLSIKPYRFDVYLTDKGYKFITISYL
DVLKKDNYYYIPEQKYDKLKLGKAIDKNAKFIASFYKNDLIKLDGEIYKIIGVNSDTRNMIE
LDLPDIRYKEYCELNNIKGEPRIKKTIGKKVNSIEKLTTDVLGNVFTNTQYTKPQLLFKRG
N*;
the amino acid sequence of SlutCas9 protein:
SEQ ID NO: 4
MRNSYILGLDIGITSVGYGIIDRVTREVIDAGVRLFPEANVENNEGRRSKRGARRLKRRRIH
RLNRIKQLLKNAGLLEGDVLPKSTNPYEIRVRGLRSPLTKDELVIALLHIAKRRGIHNINIV
GDDEETDSTLSTTAQLKKNEKALKGQFVCELQLDRLANAHQVRGEKNRFKTEDIVKEVRALL
QQQQNFHNIDNSFVEQYIALLESRRTYYEGPGEGSPYGWDGDIKKWYEMLMGYCTYFPEELR
SVKYAYTADLFNALNDLNNLVITRDDNSKLTYAEKYHIIENVFKQKKVPTLKQIAKEIGVNE
SDIKGYRINKSEKPLFTSFKLYHDLKSVFSDPTKLEDIDLLDRIAVVLTMYQDAESMKAALN
TFPEVFSEAEKEKLSALTGYAGTHRLSLKCMNLLIPDLWQTSLNQMELFVKLNLKPQKLDLS
QCHQIPTQLVDEFILSPVVKRAFTQSIKVINAIIQKYGLPDDIIIELAREKNSADKRKFLNQ
LQKKNEKARHEINTLVAQYGQPNAKRLVEKITLHQQQEGKCLYSLKDIPLEQLLKQPYLYEV
DHIIPRSVSFDNSMQNKVLVLAEENAKKGNOTPYQYLNSREASMTYPEFKQHILNLSKAKDR
ISKKKRNYLLEERDINKFDVQKDFINRNLVDTRYATRELASLLKAYFKTHELPVKVKTINGG
FTHYLRKVWKFDKDRNKGYKHHAEDALIIANADFLFKNQTLNKIEAILNEPGREVESDTVKV
QSEDNYQDLFENTKKAFAIKNFKDFKFSHRVDQKPNROLVNDTLYSTREVNEDLYVVQTLKD
IYSKDNKDVKRLFDKQPEKFLMFQHDPETFKKFELAMKQYAEEKNPLARYYEEQGYITKYAK
KGDGPPVKSLKYIGKKVGKHLDVTGDYEDSNRKLVKLSLKSFRFDIYHTDKGYKMVPITYLD
VQKKEKYYYIPTEKYEALKQEKGINQNAQFIGSFYYNDLIEFDGELYRVIGINNGDKNLVEL
DMVDIRYKEYCELNSITTTPRIVKTISPKTQSIEKYTTDILGNLYKAQPGKKPQFIFNKDE
D*;
the amino acid sequence of Sa-SauriCas9 protein:
SEQ ID NO: 5
MKRNYILGLDIGITSVGYGIIDYETRDVIDAGVRLFKEANVENNEGRRSKRGARRLKRRRRH
RIQRVKKLLFDYNLLTDHSELSGINPYEARVKGLSQKLSEEEFSAALLHLAKRRGVHNVNEV
EEDTGNELSTKEQISRNSKALEEKYVAELQLERLKKDGEVRGSINRFKTSDYVKEAKQLLKV
QKAYHQLDQSFIDTYIDLLETRRTYYEGPGEGSPFGWKDIKEWYEMLMGHCTYFPEELRSVK
YAYNADLYNALNDLNNLVITRDENEKLEYYEKFQUIENVFKQKKKPTLKQIAKEILVNEEDI
KGYRVTSTGKPEFTNLKVYHDIKDITARKEIIENAELLDQIAKILTIYQSSEDIQEELTNLN
SELTQEEIEQISNLKGYTGTHNLSLKAINLILDELWHTNDNQIAIFNRLKLVPKKVDLSQQK
EIPTTLVDDFILSPVVKRSFIQSIKVINAIIKKYGLPNDIIIELAREKNSKDAQKMINEMQK
RNAATNERIEEIIRTTGKENAKYLIEKIKLHDMQEGKCLYSLEAIPLEDLLNNPFNYEVDHI
IPRSVSFDNSFNNKVLVKQEENSKKGNRTPFQYLSSSDSKISYETFKKHILNLAKGKGRISK
TKKEYLLEERDINRFSVQKDFINRNLVDTRYATAALMNLLRSYFRVNNLDVKVKSINGGFTS
FLRRKWKFKKERNKGYKHHAEDALIIANADFIFKEWKKLDKAKKVMENQMFEEKQAESMPEI
ETEQEYKEIFITPHQIKHIKDFKDYKYSHRVDKKPNRELINDTLYSTRKDDKGNTLIVNNLN
GLYDKDNDKLKKLINKSPEKLLMYHHDPQTYQKLKLIMEQYGDEKNPLYKYYEETGNYLTKY
SKKDNGPVIKKIKYYGNKLNAHLDITDDYPNSRNKVVKLSLKSFRFDIYKCEQGYKMVSIGY
LDVLKKDNYYYIPKDKYEAEKQKKKIKESDLFVGSFYYNDLIMYEDELFRVIGVNSDINNLV
ELNMVDITYKDFCEVNNVTGEKRIKKTIGKRVVLIEKYTTDILGNLYKTPLPKKPQLIFKRG
EL*;
the amino acid sequence of Sa-SepCas9 protein:
SEQ ID NO: 6
MKRNYILGLDIGITSVGYGIIDYETRDVIDAGVRLFKEANVENNEGRRSKRGARRLKRRRRH
RIQRVKKLLFDYNLLTDHSELSGINPYEARVKGLSQKLSEEEFSAALLHLAKRRGVHNVNEV
EEDTGNELSTKEQISRNSKALEEKYVAELQLERLKKDGEVRGSINRFKTSDYVKEAKQLLKV
QKAYHQLDQSFIDTYIDLLETRRTYYEGPGEGSPFGWKDIKEWYEMLMGHCTYFPEELRSVK
YAYNADLYNALNDLNNLVITRDENEKLEYYEKFQUIENVFKQKKKPTLKQIAKEILVNEEDI
KGYRVTSTGKPEFTNLKVYHDIKDITARKEIIENAELLDQIAKILTIYQSSEDIQEELTNLN
SELTQEEIEQISNLKGYTGTHNLSLKAINLILDELWHTNDNQIAIFNRLKLVPKKVDLSQQK
EIPTTLVDDFILSPVVKRSFIQSIKVINAIIKKYGLPNDIIIELAREKNSKDAQKMINEMQK
RNAATNERIEEIIRTTGKENAKYLIEKIKLHDMQEGKCLYSLEAIPLEDLLNNPFNYEVDHI
IPRSVSFDNSFNNKVLVKQEENSKKGNRTPFQYLSSSDSKISYETFKKHILNLAKGKGRISK
TKKEYLLEERDINRFSVQKDFINRNLVDTRYATAALMNLLRSYFRVNNLDVKVKSINGGFTS
FLRRKWKFKKERNKGYKHHAEDALIIANADFIFKEWKKLDKAKKVMENQMFEEKQAESMPEI
ETEQEYKEIFITPHQIKHIKDFKDYKYSHRVDKKPNRELINDTLYSTRKDDKGNTLIVNNLN
GLYDKDNDKLKKLINKSPEKLLMYHHDPQTYQKLKLIMEQYGDEKNPLYKYYEETGNYLTKY
SKKDNGPVIKKIKYYGNKLNAHLDITDDYPNSRNKVVKLSLKNYRFDVYLTEKGYKFVTIAY
LNVFKKDNYYYIPKDMYQELKAKKKIKDTDQFIASFYKNDLIKLNGDLYKIIGVNSDDRNII
ELDYYDIKYKDYCEINNIKGEPRIKKTIGKKTESIEKLTTDVLGNLYLHSTEKAPQLIFKRG
L*;
the amino acid sequence of Sa-SeqCas9 protein:
SEQ ID NO: 7
MKRNYILGLDIGITSVGYGIIDYETRDVIDAGVRLFKEANVENNEGRRSKRGARRLKRRRRH
RIQRVKKLLFDYNLLTDHSELSGINPYEARVKGLSQKLSEEEFSAALLHLAKRRGVHNVNEV
EEDTGNELSTKEQISRNSKALEEKYVAELQLERLKKDGEVRGSINRFKTSDYVKEAKQLLKV
OKAYHOLDQSFIDTYIDLLETRRTYYEGPGEGSPFGWKDIKEWYEMLMGHCTYFPEELRSVK
YAYNADLYNALNDLNNLVITRDENEKLEYYEKFQIIENVFKQKKKPTLKQIAKEILVNEEDI
KGYRVTSTGKPEFTNLKVYHDIKDITARKEIIENAELLDQIAKILTIYQSSEDIQEELTNLN
SELTQEEIEQISNLKGYTGTHNLSLKAINLILDELWHTNDNQIAIFNRLKLVPKKVDLSQQK
EIPTTLVDDFILSPVVKRSFIQSIKVINAIIKKYGLPNDIIIELAREKNSKDAQKMINEMQK
RNAATNERIEEIIRTTGKENAKYLIEKIKLHDMQEGKCLYSLEAIPLEDLLNNPFNYEVDHI
IPRSVSFDNSFNNKVLVKQEENSKKGNRTPFQYLSSSDSKISYETFKKHILNLAKGKGRISK
TKKEYLLEERDINRFSVQKDFINRNLVDTRYATAALMNLLRSYFRVNNLDVKVKSINGGFTS
FLRRKWKFKKERNKGYKHHAEDALIIANADFIFKEWKKLDKAKKVMENQMFEEKQAESMPEI
ETEQEYKEIFITPHQIKHIKDFKDYKYSHRVDKKPNRELINDTLYSTRKDDKGNTLIVNNLN
GLYDKDNDKLKKLINKSPEKLLMYHHDPQTYQKLKLIMEQYGDEKNPLYKYYEETGNYLTKY
SKKDNGPVIKKIKYYGNKLNAHLDITDDYPNSRNKVVKLSLKPYRFDVYLDNGVYKFVTVKN
LNVIKKENYYEVNSKCYEKAKKLKKISDQAEFIASFYNNDLIKIDGELYRVIGVNTDLINRI
EVNMVDITYREYLENMNDKRSPRIFKTIASKTQSIKKYSTDILGTLYEVNSKKHPQMIMK
G*;
the amino acid sequence of Sa-ShaCas9 protein:
SEQ ID NO: 8
MKRNYILGLDIGITSVGYGIIDYETRDVIDAGVRLFKEANVENNEGRRSKRGARRLKRRRRH
RIQRVKKLLFDYNLLTDHSELSGINPYEARVKGLSQKLSEEEFSAALLHLAKRRGVHNVNEV
EEDTGNELSTKEQISRNSKALEEKYVAELQLERLKKDGEVRGSINRFKTSDYVKEAKQLLKV
QKAYHQLDQSFIDTYIDLLETRRTYYEGPGEGSPFGWKDIKEWYEMLMGHCTYFPEELRSVK
YAYNADLYNALNDLNNLVITRDENEKLEYYEKFOIIENVFKQKKKPTLKQIAKEILVNEEDI
KGYRVTSTGKPEFTNLKVYHDIKDITARKEIIENAELLDQIAKILTIYQSSEDIQEELTNLN
SELTQEEIEQISNLKGYTGTHNLSLKAINLILDELWHTNDNQIAIFNRLKLVPKKVDLSQQK
EIPTTLVDDFILSPVVKRSFIQSIKVINAIIKKYGLPNDIIIELAREKNSKDAQKMINEMQK
RNAATNERIEEIIRTTGKENAKYLIEKIKLHDMQEGKCLYSLEAIPLEDLLNNPFNYEVDHI
IPRSVSFDNSFNNKVLVKQEENSKKGNRTPFQYLSSSDSKISYETFKKHILNLAKGKGRISK
TKKEYLLEERDINRFSVQKDFINRNLVDTRYATAALMNLLRSYFRVNNLDVKVKSINGGFTS
FLRRKWKFKKERNKGYKHHAEDALIIANADFIFKEWKKLDKAKKVMENQMFEEKQAESMPEI
ETEQEYKEIFITPHQIKHIKDFKDYKYSHRVDKKPNRELINDTLYSTRKDDKGNTLIVNNLN
GLYDKDNDKLKKLINKSPEKLLMYHHDPQTYQKLKLIMEQYGDEKNPLYKYYEETGNYLTKY
SKKDNGPVIKKIKYYGNKLNAHLDITDDYPNSRNKVVKLSLKSYRFDVYLTDKGYKFVSITY
LDVLKKENYYYISEAKYDKLKLNKGIDDKAKFIGSFYYNDLIELDGEVYTVIGVNNDKNNVI
ELNLPEIRYKEYCEINNIKGSGRLRITIGKKVNSIRKLSTDVLGNRYYQSFAKKPQLVFKKG
I*;
the amino acid sequence of Sa-SlugCas9 protein:
SEQ ID NO: 9
MKRNYILGLDIGITSVGYGIIDYETRDVIDAGVRLFKEANVENNEGRRSKRGARRLKRRRRH
RIQRVKKLLFDYNLLTDHSELSGINPYEARVKGLSQKLSEEEFSAALLHLAKRRGVHNVNEV
EEDTGNELSTKEQISRNSKALEEKYVAELQLERLKKDGEVRGSINRFKTSDYVKEAKQLLKV
QKAYHQLDQSFIDTYIDLLETRRTYYEGPGEGSPFGWKDIKEWYEMLMGHCTYFPEELRSVK
YAYNADLYNALNDLNNLVITRDENEKLEYYEKFQIIENVFKQKKKPTLKQIAKEILVNEEDI
KGYRVTSTGKPEFTNLKVYHDIKDITARKEIIENAELLDQIAKILTIYQSSEDIQEELTNLN
SELTQEEIEQISNLKGYTGTHNLSLKAINLILDELWHTNDNQIAIFNRLKLVPKKVDLSQQK
EIPTTLVDDFILSPVVKRSFIQSIKVINAIIKKYGLPNDIIIELAREKNSKDAQKMINEMQK
RNAATNERIEEIIRTTGKENAKYLIEKIKLHDMQEGKCLYSLEAIPLEDLLNNPFNYEVDHU
IPRSVSFDNSFNNKVLVKQEENSKKGNRTPFQYLSSSDSKISYETFKKHILNLAKGKGRISK
TKKEYLLEERDINRFSVQKDFINRNLVDTRYATAALMNLLRSYFRVNNLDVKVKSINGGFTS
FLRRKWKFKKERNKGYKHHAEDALIIANADFIFKEWKKLDKAKKVMENQMFEEKQAESMPEI
ETEQEYKEIFITPHQIKHIKDFKDYKYSHRVDKKPNRELINDTLYSTRKDDKGNTLIVNNLN
GLYDKDNDKLKKLINKSPEKLLMYHHDPQTYQKLKLIMEQYGDEKNPLYKYYEETGNYLTKY
SKKDNGPVIKKIKYYGNKLNAHLDITDDYPNSRNKVVKLSLKPYRFDVYLTDKGYKFITISY
LDVLKKDNYYYIPEQKYDKLKLGKAIDKNAKFIASFYKNDLIKLDGEIYKIIGVNSDTRNMI
ELDLPDIRYKEYCELNNIKGEPRIKKTIGKKVNSIEKLTTDVLGNVFTNTQYTKPQLLFKRG
N*;
the amino acid sequence of Sa-SlutCas9 protein:
SEQ ID NO: 10
MKRNYILGLDIGITSVGYGIIDYETRDVIDAGVRLFKEANVENNEGRRSKRGARRLKRRRRH
RIQRVKKLLFDYNLLTDHSELSGINPYEARVKGLSQKLSEEEFSAALLHLAKRRGVHNVNEV
EEDTGNELSTKEQISRNSKALEEKYVAELQLERLKKDGEVRGSINRFKTSDYVKEAKQLLKV
QKAYHQLDQSFIDTYIDLLETRRTYYEGPGEGSPFGWKDIKEWYEMLMGHCTYFPEELRSVK
YAYNADLYNALNDLNNLVITRDENEKLEYYEKFQIIENVFKQKKKPTLKQIAKEILVNEEDI
KGYRVTSTGKPEFTNLKVYHDIKDITARKEIIENAELLDQIAKILTIYQSSEDIQEELTNLN
SELTQEEIEQISNLKGYTGTHNLSLKAINLILDELWHTNDNQIAIFNRLKLVPKKVDLSQQK
EIPTTLVDDFILSPVVKRSFIQSIKVINAIIKKYGLPNDIIIELAREKNSKDAQKMINEMQK
RNAATNERIEEIIRTTGKENAKYLIEKIKLHDMQEGKCLYSLEAIPLEDLLNNPFNYEVDHU
PRSVSFDNSFNNKVLVKQEENSKKGNRTPFQYLSSSDSKISYETFKKHILNLAKGKGRISKT
KKEYLLEERDINRFSVQKDFINRNLVDTRYATAALMNLLRSYFRVNNLDVKVKSINGGFTSF
LRRKWKFKKERNKGYKHHAEDALIIANADFIFKEWKKLDKAKKVMENQMFEEKQAESMPEIE
TEQEYKEIFITPHQIKHIKDFKDYKYSHRVDKKPNRELINDTLYSTRKDDKGNTLIVNNLNG
LYDKDNDKLKKLINKSPEKLLMYHHDPQTYQKLKLIMEQYGDEKNPLYKYYEETGNYLTKYS
KKDNGPVIKKIKYYGNKLNAHLDITDDYPNSRNKVVKLSLKSFRFDIYHTDKGYKMVPITYL
DVQKKEKYYYIPTEKYEALKQEKGINQNAQFIGSFYYNDLIEFDGELYRVIGINNGDKNLVE
LDMVDIRYKEYCELNSITTTPRIVKTISPKTQSIEKYTTDILGNLYKAQPGKKPQFIFNKDE
D*;
nucleotide sequence of sgRNA:
SEQ ID NO: 11
NNNNNNNNNNNNNNNNNNNNGUUUUAGUACUCUGGAAACAGAAUCUACUAAAACAAGGCAAA
AUGCCGUGUUUAUCUCGUCAACUUGUUGGCGAGAUUUUU;
the amino acid sequence of SlugCas9-HF protein:
SEQ ID NO: 58
MNQKFILGLDIGITSVGYGLIDYETKNIIDAGVRLFPEANVENNEGRRSKRGSRRLKRRRIH
RLERVKKLLEDYNLLDQSQIPQSTNPYAIRVKGLSEALSKDELVIALLHIAKRRGIHKIDVI
DSNDDVGNELSTKEQLNKNSKLLKDKFVCQIQLERMNEGQVRGEKNRFKTADIIKEIIQLLN
VQKNFHOLDENFINKYIELVEMRREYFEGPGKGSPYGWEGDPKAWYETLMGHCTYFPDELAS
VKYAYSADLFNALNDLNNLVIQRDGLSKLEYHEKYHIIENVFKQKKKPTLKQIANEINVNPE
DIKGYRITKSGKPQFTEFKLYHDLKSVLFDQSILENEDVLDQIAEILTIYQDKDSIKSKLTE
LDILLNEEDKENIAQLTGYTGTHRLSLKCIRLVLEEQWYSSRAQMEIFAHLNIKPKKINLTA
ANKIPKAMIDEFILSPVVKRTFGQAINLINKIIEKYGVPEDIDIELARENNSKDKQKFINEM
QKKNENTRKRINEIIGKYGNQNAKRLVEKIRLHDEQEGKCLYSLESIPLEDLLNNPNHYEVD
HIIPRSVSFDNSYHNKVLVKQSENSKKSNLTPYQYFNSGKSKLSYNQFKQHILNLSKSQDRI
SKKKKEYLLEERDINKFEVQKEFINRNLVDTRYATAELTNYLKAYFSANNMNVKVKTINGSF
TDYLRKVWKFKKERNHGYKHHAEDALIIANADFLFKENKKLKAVNSVLEKPEIESKQLDIQV
DSEDNYSEMFIIPKQVQDIKDFRNFKYSHRVDKKPNRQLINDTLYSTRKKDNSTYIVQTIKD
IYAKDNTTLKKQFDKSPEKFLMYQHDPRTFEKLEVIMKQYANEKNPLAKYHEETGEYLTKYS
KKNNGPIVKSLKYIGNKLGSHLDVTHQFKSSTKKLVKLSIKPYRFDVYLTDKGYKFITISYL
DVLKKDNYYYIPEQKYDKLKLGKAIDKNAKFIASFYKNDLIKLDGEIYKIIGVNSDTRNMIE
LDLPDIRYKEYCELNNIKGEPRIKKTIGKKVNSIEKLTTDVLGNVFTNTQYTKPQLLFKRG
N.

As described above, the present inventors have discovered a variety of Cas9 proteins that can be complexed with a single-stranded guide RNA (sgRNA). For the SauriCas9 protein, the complex thereof formed with sgRNA is referred to as CRISPR/SauriCas9 gene editing system in the present application (that is, a system in which the SauriCas9 protein and the single-stranded guide RNA (sgRNA) work together to achieve gene editing). The complexes formed by other Cas9 proteins and sgRNA can be named in a similar way, such as the CRISPR/ShaCas9 gene editing system, the CRISPR/SlugCas9 gene editing system, and so on.

All the Cas9 proteins of the present disclosure are very small, with only less than one thousand and one hundred amino acids. Particularly, SauriCas9 protein has 1061 amino acids; ShaCas9 protein, Sa-SepCas9 protein, Sa-ShaCas9 protein and Sa-SlugCas9 protein have 1055 amino acids; Sa-SeqCas9 protein has 1053 amino acids; SlugCas9 protein, SlugCas9-HF protein and SlutCas9 protein all have 1054 amino acids; and Sa-SauriCas9 and Sa-SlutCas9 proteins have 1056 amino acids.

In one embodiment, the Cas9 protein of the present disclosure has an amino acid sequence which is at least 80%, 81%, 82%, 83%, 84%, 85%, 86%, 87%, 88%, 89%, 90%, 91%, 92%, 93%, 94%, 95%, 96%, 97%, 98%, 99%, 100%, or any percentage in the range of 80%-100% identical to the amino acid sequences represented by any one of SEQ ID NOs: 1-10 and SEQ ID NO: 58.

In one embodiment, the cell comprises eukaryotic cells and prokaryotic cells. The eukaryotic cells comprise, for example, mammalian cells and plant cells. The mammalian cells comprise, for example, Chinese hamster ovary cells, baby hamster kidney cells, mouse Sertoli cells, mouse breast tumor cells, buffalo rat hepatic cells, rat hepatoma cells, SV40-transformed monkey kidney CVI lines, monkey kidney cells, canine kidney cells, human cervical cancer cells, human lung cells, human liver cells, HIH/3T3 cells, human U2-OS osteosarcoma cells, human A549 cells, human K562 cells, human HEK293 cells, human HEK293T cells, human HCT116 cells, or human MCF-7 cells or TRI cells, but are not limited to these.

In one embodiment, the CRISPR/Cas9 system comprises Staphylococcus auricularis Cas9 (SauriCas9) protein, which has an amino acid sequence represented by SEQ ID NO: 1, and works with a single-stranded guide RNA (sgRNA) to achieve gene editing.

In one embodiment, the SauriCas9 protein is derived from Staphylococcus auricularis, and has a UniProt accession number of A0A2T4M4R5.

In one embodiment, the SauriCas9 protein comprises a SauriCas9 protein that has no cleavage activity, or only has single-stranded cleavage activity, or has double-stranded cleavage activity.

In one embodiment, the CRISPR/Cas9 system comprises a Staphylococcus haemolyticus Cas9 (ShaCas9) protein, which has an amino acid sequence represented by SEQ ID NO: 2, and works with single-stranded guide RNA (sgRNA) to achieve gene editing.

In one embodiment, the ShaCas9 protein is derived from Staphylococcus haemolyticus, and has a UniProt accession number of A0A2T4SLN6.

In one embodiment, the ShaCas9 protein comprises a ShaCas9 protein that has no cleavage activity, or only has single-stranded cleavage activity, or has double-stranded cleavage activity.

In one embodiment, the CRISPR/Cas9 system comprises a Staphylococcus lugdunensis Cas9 (SlugCas9) protein, which has an amino acid sequence represented by SEQ ID NO: 3, and works with a single-stranded guide RNA (sgRNA) to achieve gene editing.

In one embodiment, the SlugCas9 protein is derived from Staphylococcus lugdunensis, and has a UniProt accession number of A0A133QCR3.

In one embodiment, the SlugCas9 protein comprises a SlugCas9 protein that has no cleavage activity, or only has single-stranded cleavage activity, or has double-stranded cleavage activity.

In one embodiment, the CRISPR/Cas9 system comprises a Staphylococcus lutrae Cas9 (SlutCas9) protein, which has an amino acid sequence represented by SEQ ID NO: 4, and works with a single-stranded guide RNA (sgRNA) to achieve gene editing.

In one embodiment, the SlutCas9 protein is derived from Staphylococcus lutrae, and has a UniProt accession number of A0A1W6BMI2.

In one embodiment, the SlutCas9 protein comprises a SlutCas9 protein that has no cleavage activity, or only has single-stranded cleavage activity, or has double-stranded cleavage activity.

In one embodiment, the CRISPR/Cas9 system comprises a Sa-SauriCas9 protein, which is a fusion protein obtained by replacing the PI domain of SaCas9 with the PI domain of SauriCas9, wherein SauriCas9 is Staphylococcus auricularis Cas9. The Sa-SauriCas9 protein has an amino acid sequence represented by SEQ ID NO: 5. The Sa-SauriCas9 fusion protein works with a single-stranded guide RNA (sgRNA) to achieve gene editing.

In one embodiment, the SauriCas9 protein is derived from Staphylococcus auricularis, and has a UniProt accession number of A0A2T4M4R5.

In one embodiment, the Sa-SauriCas9 protein comprises a Sa-SauriCas9 protein that has no cleavage activity, or only has single-stranded cleavage activity, or has double-stranded cleavage activity.

In one embodiment, the CRISPR/Cas9 system comprises a Su-SepCas9 protein, which is a fusion protein obtained by replacing the PI domain of SaCas9 with the PI domain of SepCas9, wherein SepCas9 is Staphylococcus epidermidis Cas9. The Sa-SepCas9 protein has an amino acid sequence represented by SEQ ID NO: 6. The Sa-SepCas9 fusion protein works with a single-stranded guide RNA (sgRNA) to achieve gene editing.

In one embodiment, the SepCas9 protein is derived from Staphylococcus epidermidis, and has a UniProt accession number of A0A1Q9MLU4 and a NCBI accession number of WP_075777761.1.

In one embodiment, the Sa-SepCas9 protein comprises a Sa-SepCas9 protein that has no cleavage activity, or only has single-stranded cleavage activity, or has double-stranded cleavage activity.

In one embodiment, the CRISPR/Cas9 system comprises a Sa-SeqCas9 protein, which is a fusion protein obtained by replacing the PI domain of SaCas9 with the PI domain of SeqCas9, wherein SeqCas9 is Staphylococcus equorum Cas9. The Sa-SeqCas9 protein has an amino acid sequence represented by SEQ ID NO: 7. The Sa-SeqCas9 fusion protein works with a single-stranded guide RNA (sgRNA) to achieve gene editing.

In one embodiment, the SeqCas9 protein is derived from Staphylococcus equorum, and has a UniProt accession number of A0A1E5TL62.

In one embodiment, the Sa-SeqCas9 protein comprises a Sa-SeqCas9 protein that has no cleavage activity, or only has single-stranded cleavage activity, or has double-stranded cleavage activity.

In one embodiment, the CRISPR/Cas9 system comprises a Sa-ShaCas9 protein, which is a fusion protein obtained by replacing the PI domain of SaCas9 with the PI domain of ShaCas9, wherein ShaCas9 is Staphylococcus haemolyticus Cas9. The Sa-ShaCas9 protein has an amino acid sequence represented by SEQ ID NO: 8. The Sa-ShaCas9 fusion protein works with a single-stranded guide RNA (sgRNA) to achieve gene editing.

In one embodiment, the ShaCas9 protein is derived from Staphylococcus haemolyticus, and has a UniProt accession number of A0A2T4SLN6.

In one embodiment, the Sa-ShaCas9 protein comprises a Sa-ShaCas9 protein that has no cleavage activity, or only has single-stranded cleavage activity, or has double-stranded cleavage activity.

In one embodiment, the CRISPR/Cas9 system comprises a Sa-SlugCas9 protein, which is a fusion protein obtained by replacing the PI domain of SaCas9 with the PI domain of SlugCas9, wherein SlugCas9 is Staphylococcus lugdunensis Cas9. The Sa-SlugCas9 protein has an amino acid sequence represented by SEQ ID NO: 9. The SlugCas9 fusion protein works with a single-stranded guide RNA (sgRNA) to achieve gene editing.

In one embodiment, the SlugCas9 protein is derived from Staphylococcus lugdunensis, and has a UniProt accession number of A0A133QCR3.

In one embodiment, the Sa-SlugCas9 protein comprises a Sa-SlugCas9 protein that has no cleavage activity, or only has single-stranded cleavage activity, or has double-stranded cleavage activity.

In one embodiment, the CRISPR/Cas9 system comprises a Sa-SlutCas9 protein, which is a fusion protein obtained by replacing the PI domain of SaCas9 with the PI domain of SlutCas9, wherein SlutCas9 is Staphylococcus lutrae Cas9. The Sa-SlutCas9 protein has an amino acid sequence represented by SEQ ID NO: 10. The Sa-SlutCas9 fusion protein works with a single-stranded guide RNA (sgRNA) to achieve gene editing.

In one embodiment, the SlutCas9 protein is derived from Staphylococcus lutrae, and has a UniProt accession number of A0A1W6BMI2.

In one embodiment, the Sa-SlutCas9 protein comprises a Sa-SlutCas9 protein that has no cleavage activity, or only has single-stranded cleavage activity, or has double-stranded cleavage activity.

In one embodiment, the CRISPR/Cas9 system comprises a SlugCas9-HF protein, which is an amino-acid-modified protein obtained by introducing R247A, N415A, T421A, and R656A mutations to SlugCas9. The SlugCas9-HF is Staphylococcus lugdunensis Cas9-HiFi. SlugCas9-HF works with a single-stranded guide RNA (sgRNA) to achieve gene editing. The complex of the SlugCas9-HF protein and sgRNA has a low off-target rate and high specificity, and exhibits low tolerance for non-target DNA sequences, that is, the complex is essentially incapable of or is incapable of the cleave the non-targeted DNA sequences.

In one embodiment, the SlugCas9 protein is derived from Staphylococcus lugdunensis, and has a UniProt accession number of A0A133QCR3.

In one embodiment, the SlugCas9-HF protein comprises a SlugCas9-HF protein that has no cleavage activity, or only has single-stranded cleavage activity, or has double-stranded cleavage activity.

In one embodiment, accurately locating the target DNA sequence comprises forming a complementary base pairing structure from a 20 bp or 21 bp sequence at the 5′ end of the sgRNA and the target DNA sequence.

In one embodiment, accurately locating the target DNA sequence comprises recognizing, by the complex of the Cas9 protein and the sgRNA, a PAM sequence on the target DNA sequence.

In one embodiment, a 20 bp or 21 bp sequence at the 5′ end of the sgRNA in the complex of the SlugCas9-HF protein and the sgRNA is capable of forming an imperfect complementary base pairing structure with a non-target DNA sequence. Particularly, in the present disclosure, the imperfect complementary base pairing structure comprises a part of complementary base pairing structure and a part of non-complementary base pairing structure. In a preferred embodiment, there are two or more base mismatches between the non-targeted DNA sequence and the sgRNA.

In yet another embodiment, the complex of the Cas9 protein and the sgRNA is capable of recognizing the PAM sequence on the non-target DNA sequence.

In one embodiment, the PAM sequence and the target DNA sequence are respectively as follows:
for the SauriCas9 protein, the PAM is NNGG, and the target DNA sequence is represented by SEQ ID NO: 12;
for the ShaCas9 protein, the PAM is NNGRM, and the target DNA sequence is represented by SEQ ID NO: 13;
for the SlugCas9 protein, the PAM is NNGG, and the target DNA sequence is represented by SEQ ID NO: 12;
for the SlutCas9 protein, the PAM is NNGR, and the target DNA sequence is represented by SEQ ID NO: 14;
for the Sa-SauriCas9 protein, the PAM is NNGG, and the target DNA sequence is represented by SEQ ID NO: 12;
for the Sa-SepCas9 protein, the PAM is NNGG, and the target DNA sequence is represented by SEQ ID NO: 12;
for the Sa-SeqCas9 protein, the PAM is NNGRM, and the target DNA sequence is represented by SEQ ID NO: 13;
for the Sa-ShaCas9 protein, the PAM is NNGRM, and the target DNA sequence is represented by SEQ ID NO: 13;
for the Sa-SlugCas9 protein, the PAM is NNGG, and the target DNA sequence is represented by SEQ ID NO: 12;
for the Sa-SlutCas9 protein, the PAM is NNGRR, and the target DNA sequence is represented by SEQ ID NO: 15; and
for the SlugCas9-HF protein, the PAM is NNGG, and the target DNA sequence is represented by SEQ ID NO: 12.

The nucleotide sequences of SEQ ID NOs: 12-15 are as follows:

| (SEQ ID NO: 12) | |
|---|---|
| | NNNNNNNNNNNNNNNNNNNNNNNGG; |
| (SEQ ID NO: 13) | |
| | NNNNNNNNNNNNNNNNNNNNNNNGRM; |
| (SEQ ID NO: 14) | |
| | NNNNNNNNNNNNNNNNNNNNNNNGR; |
| | and |
| (SEQ ID NO: 15) | |
| | NNNNNNNNNNNNNNNNNNNNNNNGRR; |

Those skilled in the art can understand that the base N above represents any one of the four bases, A (adenine), T (thymine), C (cytosine) and G (guanine); the base M above represents any one of the two bases, A and C; and the base R above represents any one of the two bases, A and G.

In one embodiment, the complex of the Cas9 protein and the sgRNA is capable of accurately locating the target DNA sequence, which means that the complex of the Cas9 protein and the sgRNA is capable of recognizing and binding to the target DNA sequence, or that the complex of the Cas9 protein and the sgRNA is capable of carrying a further protein fused with the Cas9 protein or a protein that specifically recognizes the sgRNA to the place where the target DNA sequence is located.

In one embodiment, the complex of the Cas9 protein and the sgRNA, or the further protein fused with the Cas9 protein, or the protein that specifically recognizes the sgRNA is capable of making modification and regulation to the target DNA region, and the modification and regulation comprises, but is not limited to, regulation of gene transcription level, DNA methylation regulation, DNA acetylation modification, histone acetylation modification, single-base editing, or chromatin imaging tracking.

In one embodiment, the complex of the SlugCas9-HF protein and the sgRNA has low tolerance for the non-target DNA sequence. That is, the complex of the SlugCas9-HF protein and the sgRNA is essentially incapable of or is incapable of recognizing and binding to the non-target DNA sequence, or the complex of the SlugCas9-HF protein and the sgRNA is essentially incapable of or is incapable of carrying the further protein fused with the SlugCas9-HF protein or a protein that specifically recognizes the sgRNA to the place where the non-target DNA sequence is located.

In the context of the present disclosure, the term “essentially” in the expression “the complex of the SlugCas9-HF protein and the sgRNA is essentially incapable of recognizing and binding to the non-targeted DNA sequences” means that there is little or no biological and/or statistical significance for the extent of recognition and binding, if any, of the complex of the SlugCas9-HF protein and the sgRNA to the non-targeted DNA sequences.

In yet another embodiment, the complex of the SlugCas9-HF protein and the sgRNA, or the other protein fused with the SlugCas9-HF protein, or the protein that specifically recognizes the sgRNA is essentially incapable of or is incapable of making modification and regulation to the non-targeted DNA region, and the modification and regulation comprises, but is not limited to, regulation of gene transcription level, DNA methylation regulation, DNA acetylation modification, histone acetylation modification, single-base editing, or chromatin imaging tracking.

Similarly, in the context of the present disclosure, the term “essentially” in the expression “the complex of the SlugCas9-HF protein and the sgRNA, or the other protein fused with the SlugCas9-HF protein, or the protein that specifically recognizes the sgRNA is essentially incapable of or is incapable of making modification and regulation to the non-targeted DNA region” means that there is little or no biological and/or statistical significance for the extent of modification and regulation, if any, of the complex of the SlugCas9-HF protein and sgRNA, or the other protein fused with the SlugCas9-HF protein, or the protein that specifically recognizes the sgRNA to the non-targeted DNA region.

In one embodiment, the single-base editing comprises, but is not limited to, conversion of adenine to guanine, or conversion of cytosine to thymine, or conversion of cytosine to uracil, or conversion between other bases.

The CRISPR/Cas9 gene editing system of the present disclosure has high editing activity and high specificity, and shows significant advantages compared with the existing CRISPR/Cas9 gene editing systems.

In the present disclosure, the editing efficiency and off-target rate of the CRISPR/Cas9 system are detected by technologies such as gene synthesis, molecular cloning, cell transfection, PCR product deep sequencing, flow cytometry analysis technology, bioinformatics analysis and the like.

The CRISPR/Cas9 gene editing system of the present disclosure is verified in a GFP-reporter cell line containing target sites, and it is found that the gene editing system can edit target genes with high specificity and a low off-target rate.

Thus, in the second aspect, the present disclosure provides a method for gene editing in cells with the CRISPR/Cas9 system for gene editing according to the first aspect of the present disclosure, wherein the method edits a target DNA sequence by recognizing and locating the target DNA sequence with the complex of a Cas9 protein and a sgRNA, and the method comprises the steps of:
(1) synthesizing a Cas9 gene sequence and cloning it into an expression vector such as pAAV2_ITR, to obtain an expression vector cloned with the Cas9 gene sequence, such as pAAV2_Cas9_ITR, wherein the Cas9 gene sequence:
   (a) has a nucleotide sequence encoding an amino acid sequence represented by any one of SEQ ID NOs: 1-10 and SEQ ID NO: 58;
   (b) has a nucleotide sequence encoding an amino acid sequence that is at least 80% identical to the amino acid sequence represented by any one of SEQ ID NOs: 1-10 and SEQ ID NO: 58; or
   (c) is a humanized Cas9 gene sequence, for example, the one having a nucleotide sequence represented by any one of SEQ ID NOs: 23-32 and SEQ ID NO: 112;
(2) synthesizing oligo single-stranded DNAs corresponding to the sgRNA, i.e., an oligo forward-strand sequence (Oligo-F) and an oligo reverse-strand sequence (Oligo-R), and annealing and ligating the oligo forward-strand sequence and the oligo reverse-strand sequence to a restriction site, such as the BsaI digestion site of plasmid pAAV2_Cas9_U6_BsaI, of the expression vector cloned with the Cas9 gene sequence to obtain an expression vector, such as pAAV2_Cas9-hU6-sgRNA, for expressing the Cas9 protein and the sgRNA; wherein the sgRNA has a nucleotide sequence represented by SEQ ID NO: 11, or a nucleotide sequence that is at least 80% identical to the nucleotide sequence represented by SEQ ID NO: 11, or a modification comprising, for example, phosphorylation, truncation, addition, sulfurization, methylation or hydroxylation, based on the nucleotide sequence represented by SEQ ID NO: 11; and
(3) delivering the expression vector expressing the Cas9 protein and the sgRNA into cells comprising a target site to edit the target site.

SEQ ID NOs: 23-32 and SEQ ID NO: 112 are as follows.

the humanized SauriCas9 gene sequence:
SEQ ID NO: 23
ATGCAGGAGAACCAGCAGAAGCAGAACTACATCCTGGGCCTGGACATCGG
AATCACCAGCGTCGGCTACGGACTGATCGATAGCAAGACAAGAGAAGTGA
TCGACGCCGGCGTTAGACTCTTTCCAGAAGCTGATAGCGAGAACAACTCC
AACCGCAGAAGCAAGCGGGGCGCCAGACGGTTAAAACGGAGAAGAATCCA
CCGGCTGAACCGGGTCAAAGACCTGCTCGCTGATTACCAGATGATCGATC
TTAACAATGTTCCTAAGAGCACCGACCCCTACACCATCAGAGTGAAGGGC
CTCCGGGAGCCTCTGACAAAAGAAGAATTCGCCATCGCCCTCCTGCATAT
CGCTAAGAGAAGAGGCCTGCACAACATCAGTGTGTCCATGGGCGACGAAG
AGCAGGACAATGAACTGAGCACCAAGCAGCAGCTGCAAAAGAATGCCCAG
CAACTGCAGGACAAGTATGTGTGCGAACTGCAGTTAGAACGGCTGACCAA
CATCAACAAGGTCAGAGGCGAGAAGAACAGATTTAAGACAGAGGACTTTG
TGAAAGAAGTGAAACAGCTGTGCGAAACCCAGAGACAGTACCACAACATC
GACGACCAATTCATCCAGCAGTACATCGACCTGGTGTCTACAAGACGGGA
GTACTTCGAGGGCCCCGGCAACGGCTCTCCATACGGCTGGGACGGCGACC
TGCTGAAGTGGTACGAGAAGCTGATGGGCAGATGCACCTATTTCCCCGAA
GAACTGAGGTCCGTGAAGTACGCCTACAGCGCCGACCTCTTCAACGCCCT
GAACGACCTGAACAACCTCGTTGTGACCAGGGATGACAATCCAAAGCTTG
AGTACTACGAGAAGTACCACATTATTGAGAACGTGTTCAAGCAAAAGAAG
AATCCCACACTCAAACAAATCGCCAAAGAGATCGGCGTGCAAGATTACGA
CATCCGGGGCTATAGAATCACAAAGAGCGGCAAACCTCAGTTCACCTCTT
TTAAGCTGTATCACGACCTGAAGAACATCTTCGAGCAGGCCAAATACCTG
GAAGATGTGGAAATGCTGGACGAGATCGCCAAGATCCTGACCATCTACCA
GGATGAGATTAGCATCAAGAAAGCCCTGGACCAGCTGCCCGAACTGCTGA
CAGAGAGCGAGAAATCTCAGATCGCACAGCTCACCGGCTATACAGGCACC
CACAGACTGAGCCTGAAGTGCATCCACATTGTGATCGACGAGCTGTGGGA
GAGCCCCGAGAACCAGATGGAAATCTTTACCAGACTGAATCTGAAACCTA
AGAAGGTGGAAATGAGCGAGATCGACAGCATACCCACCACCCTGGTCGAC
GAGTTCATCCTCTCACCTGTGGTGAAGCGGGCCTTCATCCAGAGCATCAA
GGTAATCAACGCAGTGATCAATCGGTTCGGCCTGCCAGAGGACATCATCA
TCGAGCTGGCCAGAGAAAAGAATAGCAAGGATCGGAGAAAGTTCATTAAC
AAGCTGCAGAAACAAAATGAGGCCACAAGAAAGAAAATCGAACAGCTGCT
GGCCAAGTACGGCAACACCAATGCCAAGTACATGATCGAGAAGATCAAGC
TGCACGACATGCAGGAGGGCAAGTGCCTGTACAGCCTGGAGGCTATTCCT
CTGGAAGACCTGCTGAGCAACCCGACACACTACGAAGTTGACCACATTAT
CCCCAGATCTGTGAGCTTTGACAACAGCCTGAACAACAAAGTGCTGGTGA
AACAAAGCGAAAACAGCAAGAAGGGCAATCGCACCCCTTACCAGTACCTG
AGCAGCAACGAGTCTAAGATTAGCTACAACCAGTTTAAGCAGCACATCCT
GAACCTGAGCAAGGCCAAGGACAGAATCAGCAAGAAAAAAAGAGATATGC
TGCTGGAAGAGAGAGATATCAACAAGTTCGAAGTGCAGAAGGAATTCATT
AACCGGAACCTGGTGGATACACGGTACGCCACCAGAGAACTGTCTAACCT
GCTGAAGACCTACTTCAGCACCCATGACTACGCCGTGAAGGTGAAGACCA
TCAACGGCGGCTTCACTAACCACCTGAGGAAGGTGTGGGATTTCAAGAAG
CACAGAAACCACGGCTACAAGCACCACGCCGAAGATGCCCTGGTGATCGC
CAACGCCGACTTCCTGTTTAAGACACATAAGGCCCTGCGGAGAACCGATA
AGATCCTGGAACAACCTGGCCTGGAAGTGAATGATACAACCGTGAAAGTG
GACACCGAGGAAAAATACCAGGAGCTGTTCGAGACACCTAAGCAAGTGAA
GAACATCAAGCAGTTCCGGGACTTCAAGTACAGCCACCGAGTGGACAAGA
AGCCTAACCGGCAGCTTATCAACGACACACTGTACTCCACCAGAGAGATT
GATGGCGAAACCTACGTGGTGCAGACCCTTAAGGATCTGTACGCCAAGGA
CAACGAGAAAGTGAAGAAGCTGTTCACCGAAAGACCTCAGAAGATCCTGA
TGTACCAGCACGACCCTAAGACCTTCGAGAAACTGATGACAATCCTGAAC
CAGTACGCTGAGGCCAAGAACCCTCTGGCTGCTTATTACGAGGACAAAGG
CGAGTACGTGACCAAGTACGCCAAGAAAGGCAATGGACCTGCCATCCACA
AGATCAAGTATATCGATAAGAAGCTTGGATCTTACCTGGATGTTAGCAAC
AAGTATCCTGAGACACAGAACAAGCTTGTGAAGCTGTCCCTGAAGAGCTT
TAGATTCGACATCTACAAGTGTGAACAGGGCTACAAGATGGTGTCCATCG
GATACCTGGACGTGCTGAAGAAAGATAACTACTACTACATCCCTAAGGAC
AAGTACGAGGCCGAGAAGCAGAAAAAGAAGATCAAGGAATCTGATCTTTT
TGTGGGCAGCTTCTACTACAACGACCTCATCATGTACGAGGATGAACTGT
TCAGAGTGATAGGAGTGAACAGCGACATCAACAATCTGGTTGAGCTAAAC
ATGGTCGACATTACCTACAAGGACTTCTGCGAGGTGAACAACGTGACAGG
CGAGAAAAGAATCAAAAAGACTATCGGCAAGCGCGTGGTCCTGATCGAGA
AGTACACCACAGATATTCTAGGCAACCTGTACAAGACTCCCCTGCCTAAG
AAGCCCCAGCTTATCTTCAAGCGGGGAGAACTG;
the humanized ShaCas9 gene sequence:
SEQ ID NO: 24
ATGAAGACAGATTACATCCTGGGCCTGGATATCGGCATCACCAGCGTGGG
CTATGGCATCATCAACTACAATGACAAGAGCATCATCGACGCCGGAGTGC
GGCTGTTCCCCGAGGCCAATGTTGAAAACAACGAGGGCAGAAGAAGCAAG
AGAGGCGCCAGAAGGCTGAAGAGACGGCGGATTCACCGGCTGGAAAGAGT
GAAGCAGCTTCTGCTGGACTACAAGCTGCTGGACAGCATCGACGTGATCC
CTCAGTCTACCAACCCCTACGAGATCCGGGTGCGGGGCCTGAGAGAGAAG
CTGACCAAAGACGAGCTGGTGATCGCGCTGCTCCATTTGGCTAAGCGCCG
TGGAATTCACAACATCGACGTGATCGACCAGGAGGAGGATGCCAGCAACG
AGCTGAGTACAAAGGAGCAGCTGAGCAAGAACAATCTGCTGCTGAGAGAT
AAGAGCATCTGTGAAGTCCTGCTGGAACGGTACAACGAGGGCAAGGTGCG
CGGAGAGAAAAACAGATTCAAAACATCTGATATCGTGAACGAGATCAGAC
AGATTCTGGAGACACAGAAGGAAGTGCACCACCTGGACGATAGCTTCATC
GACAAGTACATCGAACTGGTGGAAACCCGAAGAGAATATTACGAGGGCCC
TGGCGAAGGAAGTCCTTACGGCTGGGGAGCCGATCTGAAGAAGTGGTACG
AGCACCTGATGGGCCGGTGCACCTACTTCCCTGAGGAACTGAGATCCGTG
AAGTACGCCTACTCAGCCGACCTGTTCAATGCCCTGAACGACCTGAACAA
CCTGGTTATCCAGAGAGACGGCCTGAACAAGCTGGAATATCACGAGAAGT
ACCACATCATTGAAAACGTGTTCAAGCAGAAAAAGAAACCTACACTGAAG
CAGATCAGCAACGAGATCGGCGTGAACCCTGAAGACATCAAGGGCTACCG
GATCACCAAATCTGGCAAGGAGAATTTCACCGAGTTCAAGTTGTACCACG
ATCTCAAGAAGATCCTGAAAGACCAGAGCATCCTAGAAAATATCCAGCTG
CTGGACCAGATCGCCGAGATCATCACAATCTACCAGGACAAGGAGAGCAT
TAAAAAAGAACTGGAACAGCTCACCGAGCCTATAAATGAAATCGACAAGG
AATCTATTTCTGACCTGGCTGGATACAACGGCACCCACAGACTTTCTCTG
AAGTGCATCAACCTGGTGCTGGAAGAACTGTGGCACACCAGCAGAAATCA
AATGGAAATCTTCGCCTACCTGAACATCAAACCCAGAAAGATCGACCTGC
AGAAAGCTAATAAAATCCCCAAGGACATGATCGACGAATTCATCCTGAGC
CCTGTGGTGAAGCGAACATTCGGCCAGGCCATCAACGTGATTAACAAGGT
GATTGAGAAGTACGGCGTTCCTAAGGACATCATCATCGAGCTCGCCAGAG
AGAACAACACCAAGGACAAGCAGAAATTCATTAACGAACTCCAGAAGAAA
AACGAGAAAACCAGACAGCGGATCAATGAGATCATCGGAAAGACCGGCAA
CCAGAACGCCAAGAGACTGGTGGAAAAGATCAGACTGCACGACGAGCAGG
AGGGAAAATGCCTGTACAGCCTGGAGAGCATCCCACTGGAGGACCTCCTC
AACAACCCCAACCACTACGAGGTGGACCACATCATACCTAGAAGCGTGTC
TTTTGATAACAGTTATCAAAACAAGGTGCTCGTCAAGCAAACAGAAAACA
GCAAGAAGGGGAATAGAACACCTTACCAGTACCTGAACAGCGGCGAGGCT
AAGCTGAGCTACAACCAGTTCAAGCAACACGTGCTGAACCTGAGCAAGAG
CAAGGATAGAATTTCCAAGAAGAAGAAAGAGTACCTCCTGGAGGAACGGG
ACATTAACAAGTTCGAGGTGCAGAAGGAATTCATCAACAGAAACCTGGTC
GACACCCGATATGCCACCCGCGAGCTGACCAATTACCTGAAGGCCTATTT
CAGCGCTAACGATATGGACGTGAAGGTCAAGACCATCAATGGAAGCTTTA
CCAACCACCTGCGGAAGGTTTGGGACTTCAACAAGGAAAGAAACCACGGC
TACAAACACCACGCCGAGGATGCCCTGATCATCGCCAATGCCGACTTCCT
GTTTAAGGAGAACAAGAAGCTGAAGGCCGCCAACAAAGTGCTGGAACAGC
CTGAGAGAAAGGAAATTGAAACGAAGATCGAGGTGAATTCCGATGAGAAT
TACCAAGATCTCTTTGTGATCCCCCAGCAGGTGAAAGAAATCAAGGAGTT
TAGAGATTTTAAGTACAGCCACAGAGTGGACAAAAAACCTAATAGACAGC
TGATCAACGATACACTGTACTCCACAAGAGTGAAGGACGACGACCTGTAC
ATCATATCTCCTATCAAGAACATCTACAGCAAAGACAACACAGATCTGAA
GAAGCACTTCAACAAAAACCCAGAGAAGTTCCTGATGTACCAGAAAGACC
CCAAGACCTTCGAGAAACTGGAAGTTATTATGAAGCAGTACGCCAACGAG
AAGAATCCTCTGGCCAAATACCACGAGGAAACAGGCGAGTACCTGACGAA
GTACAGCAAGACCAACAACGGGCCAATCGTGAAAAGCATTAAGATGTTCG
GCAAGAAAGTGGGCAATCACCTGGATGTGACCAACCAGTACAACAATAGC
CGGAACAAGCTGGTGAAGCTGAGCTTCAAAAGCTACAGATTCGACGTGTA
CCTGACAGACAAGGGCTACAAGTTCGTGTCCATCACCTACCTGGACGTGC
TGAAGAAAGAGAATTACTACTACATCAGCGAGGCCAAGTACGACAAACTG
AAACTAAACAAGGGCATCGATGACAAGGCCAAGTTCATCGGCAGCTTCTA
CTACAACGACCTGATCGAGCTGGACGGCGAGGTGTACACCGTGATCGGCG
TGAATAATGACAAGAACAACGTCATCGAGCTCAATCTCCCCGAAATTAGA
TACAAGGAATACTGCGAGATTAACAACATCAAGGGCTCCGGCAGACTCCG
GATCACCATCGGCAAGAAGGTGAACTCCATTCGGAAGCTGTCCACCGACG
TGCTCGGCAACCGGTACTACCAGTCTTTCGCCAAAAAGCCTCAGCTCGTG
TTCAAGAAGGGAATA;
the humanized SlugCas9 gene sequence:
SEQ ID NO: 25
ATGAACCAAAAATTCATACTGGGACTGGACATCGGAATCACCAGCGTGGG
CTACGGCCTGATCGACTACGAGACAAAGAATATCATCGATGCCGGCGTTA
GACTGTTCCCCGAGGCCAACGTGGAAAACAACGAGGGAAGAAGGTCCAAA
CGTGGAAGCAGAAGACTGAAGCGACGCCGCATTCACAGACTTGAACGGGT
GAAGAAGCTGCTCGAGGATTATAATCTGCTGGATCAGTCCCAGATTCCTC
AGTCTACAAACCCCTACGCCATCCGCGTGAAGGGCCTGTCTGAAGCCCTG
AGCAAGGACGAACTCGTGATTGCCCTGCTCCATATCGCCAAGAGAAGAGG
CATCCACAAGATCGACGTGATCGACAGCAACGACGACGTGGGGAACGAGC
TCAGCACCAAGGAACAGCTGAATAAGAACAGCAAGCTGCTGAAAGACAAA
TTTGTGTGCCAGATCCAGCTGGAAAGAATGAATGAGGGCCAGGTGCGGGG
AGAGAAAAACCGGTTCAAGACCGCTGATATCATCAAGGAAATCATCCAGC
TGCTGAATGTGCAGAAGAACTTCCACCAGCTGGACGAGAACTTCATCAAC
AAGTACATCGAACTGGTTGAGATGAGACGGGAATACTTCGAGGGCCCCGG
CAAGGGCAGTCCATATGGCTGGGAAGGCGACCCTAAGGCTTGGTACGAGA
CACTGATGGGCCACTGCACCTACTTCCCAGATGAGCTGAGAAGCGTGAAA
TACGCCTACAGTGCCGACCTGTTCAACGCTCTGAACGACCTGAACAACCT
GGTCATCCAAAGAGATGGACTGTCTAAGCTCGAGTATCATGAGAAGTATC
ACATCATCGAGAACGTGTTCAAGCAGAAGAAGAAACCTACACTGAAGCAG
ATCGCCAATGAGATCAATGTCAACCCTGAAGATATCAAGGGCTACAGAAT
CACAAAGTCTGGCAAGCCCCAGTTTACCGAGTTTAAGCTCTACCACGACC
TGAAAAGCGTGCTGTTTGACCAGAGCATCCTGGAGAACGAAGACGTGCTG
GACCAGATCGCTGAGATCCTGACCATCTACCAGGACAAGGATAGCATCAA
ATCTAAGCTGACGGAACTGGACATCCTGCTGAACGAGGAAGATAAGGAAA
ACATCGCCCAGCTGACTGGCTACACCGGGACCCACCGGCTCAGCCTGAAA
TGCATCCGGCTGGTCCTGGAAGAGCAGTGGTATTCTAGCCGGAATCAGAT
GGAAATCTTCACACACCTGAACATTAAGCCTAAGAAGATCAACCTGACAG
CCGCCAACAAGATCCCGAAGGCTATGATCGACGAGTTCATCCTGAGCCCT
GTGGTGAAGAGGACCTTCGGCCAGGCCATTAACCTTATTAACAAGATCAT
AGAAAAGTACGGCGTGCCTGAAGATATCATCATCGAGCTGGCCAGAGAAA
ATAATAGCAAGGACAAGCAGAAGTTCATCAATGAGATGCAGAAAAAGAAC
GAGAACACCAGAAAGAGAATTAACGAAATCATCGGCAAGTATGGCAACCA
GAACGCCAAGAGACTGGTCGAGAAGATTAGACTGCACGACGAGCAGGAGG
GCAAGTGCCTGTACTCACTGGAAAGCATCCCTCTGGAGGACCTGCTGAAC
AACCCCAACCACTACGAGGTGGACCACATCATTCCAAGATCTGTGTCCTT
CGACAACTCTTACCACAACAAAGTGCTCGTGAAGCAGAGCGAGAACTCCA
AAAAATCCAACCTGACCCCTTACCAGTACTTTAACAGCGGCAAGTCCAAG
CTCTCTTACAACCAGTTTAAACAACACATCCTGAACCTGAGCAAGTCCCA
GGATAGAATCAGCAAAAAAAAGAAAGAGTATCTGCTGGAAGAACGGGACA
TCAACAAGTTCGAGGTGCAAAAAGAGTTCATCAATAGAAACCTGGTGGAT
ACCCGGTACGCCACAAGAGAGCTGACAAACTACCTGAAGGCCTACTTCAG
CGCCAACAATATGAACGTGAAGGTGAAAACGATCAACGGCAGCTTCACCG
ATTACCTGCGGAAAGTGTGGAAGTTTAAGAAGGAACGGAACCACGGCTAC
AAGCACCACGCCGAGGACGCCCTGATTATCGCTAATGCCGATTTCCTGTT
CAAAGAGAACAAGAAGCTGAAAGCCGTGAACTCTGTGCTGGAAAAACCTG
AGATCGAGAGCAAGCAGCTGGATATCCAGGTGGATAGCGAGGATAACTAC
AGCGAAATGTTCATCATCCCTAAGCAGGTCCAGGACATCAAGGACTTCAG
AAACTTCAAGTACAGCCACAGAGTGGACAAGAAGCCTAACAGACAGCTGA
TCAACGATACACTGTACAGCACCCGGAAGAAGGACAACTCCACCTACATC
GTGCAGACCATCAAAGATATCTATGCCAAAGATAATACCACCCTGAAGAA
GCAGTTTGACAAGTCACCCGAGAAGTTCCTCATGTACCAACACGATCCGC
GGACCTTCGAGAAGTTGGAAGTGATCATGAAGCAGTACGCTAATGAGAAG
AATCCTCTGGCCAAGTACCACGAGGAAACAGGCGAGTACCTGACCAAATA
CAGCAAAAAAAACAACGGCCCTATCGTGAAAAGCCTGAAGTACATTGGAA
ACAAGCTGGGCAGCCACCTAGATGTGACCCACCAGTTCAAGAGCAGCACC
AAGAAGTTGGTGAAGCTGAGCATCAAGCCTTATAGATTCGACGTCTACCT
GACCGACAAGGGATATAAGTTCATCACCATCAGCTACCTGGACGTGCTGA
AGAAAGACAATTACTACTACATACCCGAACAGAAGTACGACAAGCTCAAA
CTGGGCAAGGCCATCGACAAAAACGCCAAGTTTATCGCTAGCTTCTACAA
GAATGATCTGATCAAGCTGGACGGCGAGATCTACAAGATCATCGGCGTGA
ATAGCGACACCAGAAACATGATCGAACTGGATCTGCCTGACATCAGATAC
AAAGAATACTGCGAGCTGAACAATATCAAGGGCGAACCTAGAATCAAAAA
GACCATCGGCAAAAAGGTGAATAGCATCGAAAAACTGACAACCGACGTGC
TGGGCAACGTGTTCACCAACACCCAGTACACAAAACCTCAGCTGCTGTTC
AAGCGAGGAAAT;
the humanized SlutCas9 gene sequence:
SEQ ID NO: 26
ATGAGAAACAGCTACATCCTGGGCCTGGACATCGGAATCACCAGCGTGGG
ATATGGCATCATCGACAGAGTCACCAGAGAGGTGATCGACGCCGGCGTGC
GGCTTTTCCCCGAGGCCAACGTGGAGAACAACGAGGGCAGACGGAGCAAG
AGAGGAGCCCGGCGGCTGAAAAGAAGGCGGATCCACCGGCTGAATAGAAT
CAAGCAGCTGCTGAAAAACGCCGGCCTGCTGGAGGGAGATGTGCTGCCTA
AGTCTACCAACCCCTATGAGATCAGAGTGCGGGGCCTCCGAAGCCCTCTG
ACCAAAGATGAACTGGTGATCGCCCTGCTCCACATCGCCAAAAGAAGAGG
CATCCACAACATCAACATCGTGGGAGATGACGAAGAAACGGACAGCACAC
TGAGTACCACAGCCCAGCTGAAGAAGAACGAGAAGGCTCTCAAGGGACAG
TTTGTTTGTGAACTGCAACTGGACAGACTGGCTAATGCCCACCAAGTGCG
GGGCGAGAAAAATCGATTTAAGACAGAGGACATTGTGAAGGAAGTCAGAG
CCCTGCTTCAGCAACAGCAGAACTTCCACAACATCGATAATTCTTTCGTG
GAACAGTACATTGCCCTGCTGGAGAGCCGGAGGACCTACTACGAGGGCCC
TGGCGAAGGCTCTCCTTACGGCTGGGACGGCGACATTAAGAAGTGGTACG
AGATGCTGATGGGCTATTGCACCTACTTCCCTGAAGAGCTGAGAAGCGTG
AAGTACGCCTACACCGCCGATCTGTTCAACGCCCTGAATGACCTGAACAA
CCTGGTGATCACCCGGGACGACAACAGCAAATTGACATACGCCGAGAAGT
ACCATATCATCGAGAACGTGTTCAAACAGAAGAAAGTACCTACACTGAAG
CAGATCGCCAAGGAAATCGGAGTGAACGAGAGCGATATTAAGGGCTACAG
AATCAACAAATCTGAGAAACCTCTGTTCACCAGCTTCAAACTGTACCACG
ATCTGAAGAGCGTGTTCAGCGACCCTACAAAACTGGAAGATATCGACTTG
CTGGACCGAATCGCCGTGGTGCTGACCATGTACCAAGATGCCGAATCCAT
GAAAGCCGCCCTGAACACCTTCCCTGAAGTGTTCAGCGAAGCAGAGAAAG
AGAAGCTGAGCGCCCTCACAGGCTACGCTGGCACCCATAGACTGTCTCTG
AAGTGCATGAACCTGCTGATCCCTGATCTGTGGCAGACAAGCCTGAACCA
GATGGAACTGTTCGTGAAGCTGAATCTGAAACCACAGAAGCTGGACCTGA
GCCAGTGCCACCAGATTCCTACCCAGCTGGTGGACGAGTTCATCCTGTCT
CCTGTGGTGAAAAGAGCCTTCACCCAAAGCATCAAGGTGATCAACGCCAT
CATCCAGAAATACGGCCTGCCCGACGACATCATAATCGAGCTGGCCAGGG
AAAAAAACAGCGCCGATAAGCGGAAGTTCCTGAATCAGCTGCAGAAGAAG
AACGAGAAGGCCCGGCACGAGATCAATACCCTGGTGGCCCAGTACGGCCA
GCCAAATGCTAAGCGGCTGGTGGAAAAGATCACACTGCACCAGCAGCAGG
AGGGCAAGTGTCTGTACTCCCTGAAGGATATCCCCCTGGAGCAGCTGCTG
AAGCAGCCCTACCTGTACGAGGTGGACCACATCATCCCCAGAAGCGTTTC
TTTCGACAACAGCATGCAGAACAAGGTGCTGGTCCTGGCCGAAGAAAACG
CCAAAAAGGGCAACCAGACCCCTTACCAGTACCTGAATAGCAGAGAGGCC
AGCATGACCTACCCCGAATTCAAACAGCACATCCTGAATCTGAGCAAGGC
CAAGGACCGGATCAGCAAGAAGAAGCGGAACTACCTGCTCGAGGAAAGAG
ATATCAACAAGTTCGACGTGCAAAAGGACTTCATCAACAGAAACCTGGTT
GACACCAGATACGCCACCAGAGAGCTCGCCTCTCTGCTGAAGGCTTATTT
CAAGACACATGAGCTCCCTGTGAAAGTGAAGACCATCAACGGCGGATTTA
CCCACTACCTGAGAAAGGTGTGGAAGTTTGACAAAGATAGAAACAAGGGC
TACAAGCACCACGCCGAGGATGCACTGATCATCGCCAACGCCGACTTTCT
GTTTAAGAACCAGACTCTGAACAAAATCGAGGCTATCCTGAACGAGCCCG
GCAGAGAGGTGGAATCTGACACAGTGAAGGTGCAGAGCGAGGACAATTAC
CAGGACCTGTTTGAGAACACCAAGAAGGCTTTCGCCATCAAGAATTTCAA
GGATTTCAAGTTTAGCCACAGAGTGGACCAGAAGCCTAACCGGCAGCTCG
TGAACGACACCCTGTACAGCACCAGAGAGGTGAACGAAGATCTGTACGTG
GTGCAGACCCTGAAGGACATCTACAGCAAGGACAACAAAGACGTGAAGCG
GCTCTTCGACAAGCAACCCGAGAAGTTCCTGATGTTCCAGCACGACCCCG
AGACATTCAAGAAATTCGAGCTGGCTATGAAGCAATATGCCGAAGAGAAG
AACCCTCTGGCTAGATACTACGAGGAACAGGGCTACATCACCAAGTACGC
CAAGAAAGGAGACGGCCCACCTGTGAAAAGCCTCAAATACATCGGCAAGA
AGGTGGGAAAACACCTGGATGTGACCGGCGACTACGAGGATAGCAACCGG
AAGCTGGTGAAGCTGAGCCTGAAGTCCTTTAGATTCGATATCTACCACAC
AGACAAGGGCTACAAGATGGTTCCAATCACTTACCTGGATGTGCAGAAAA
AAGAGAAGTACTACTACATCCCCACCGAGAAATACGAAGCCCTGAAGCAG
GAGAAGGGCATCAACCAGAATGCTCAGTTCATTGGCAGCTTCTACTACAA
CGACCTGATCGAGTTCGATGGCGAGCTGTATAGAGTGATCGGCATCAACA
ACGGCGACAAGAATCTCGTTGAACTCGACATGGTGGACATTAGATATAAG
GAATACTGCGAGCTGAACTCCATCACCACCACACCTAGAATCGTTAAGAC
CATCAGCCCCAAGACCCAGAGCATCGAGAAGTACACAACAGATATCCTGG
GCAACCTGTACAAAGCCCAGCCTGGCAAGAAGCCTCAGTTCATCTTCAAC
AAAGACGAGGAT;
the humanized Sa-SauriCas9 gene sequence:
SEQ ID NO: 27
ATGAAGCGGAACTACATCCTGGGCCTGGACATCGGCATCACCAGCGTGGG
CTACGGCATCATCGACTACGAGACACGGGACGTGATCGATGCCGGCGTGC
GGCTGTTCAAAGAGGCCAACGTGGAAAACAACGAGGGCAGGCGGAGCAAG
AGAGGCGCCAGAAGGCTGAAGCGGCGGAGGCGGCATAGAATCCAGAGAGT
GAAGAAGCTGCTGTTCGACTACAACCTGCTGACCGACCACAGCGAGCTGA
GCGGCATCAACCCCTACGAGGCCAGAGTGAAGGGCCTGAGCCAGAAGCTG
AGCGAGGAAGAGTTCTCTGCCGCCCTGCTGCACCTGGCCAAGAGAAGAGG
CGTGCACAACGTGAACGAGGTGGAAGAGGACACCGGCAACGAGCTGTCCA
CCAAAGAGCAGATCAGCCGGAACAGCAAGGCCCTGGAAGAGAAATACGTG
GCCGAACTGCAGCTGGAACGGCTGAAGAAAGACGGCGAAGTGCGGGGCAG
CATCAACAGATTCAAGACCAGCGACTACGTGAAAGAAGCCAAACAGCTGC
TGAAGGTGCAGAAGGCCTACCACCAGCTGGACCAGAGCTTCATCGACACC
TACATCGACCTGCTGGAAACCCGGCGGACCTACTATGAGGGACCTGGCGA
GGGCAGCCCCTTCGGCTGGAAGGACATCAAAGAATGGTACGAGATGCTGA
TGGGCCACTGCACCTACTTCCCCGAGGAACTGCGGAGCGTGAAGTACGCC
TACAACGCCGACCTGTACAACGCCCTGAACGACCTGAACAATCTCGTGAT
CACCAGGGACGAGAACGAGAAGCTGGAATATTACGAGAAGTTCCAGATCA
TCGAGAACGTGTTCAAGCAGAAGAAGAAGCCCACCCTGAAGCAGATCGCC
AAAGAAATCCTCGTGAACGAAGAGGATATTAAGGGCTACAGAGTGACCAG
CACCGGCAAGCCCGAGTTCACCAACCTGAAGGTGTACCACGACATCAAGG
ACATTACCGCCCGGAAAGAGATTATTGAGAACGCCGAGCTGCTGGATCAG
ATTGCCAAGATCCTGACCATCTACCAGAGCAGCGAGGACATCCAGGAAGA
ACTGACCAATCTGAACTCCGAGCTGACCCAGGAAGAGATCGAGCAGATCT
CTAATCTGAAGGGCTATACCGGCACCCACAACCTGAGCCTGAAGGCCATC
AACCTGATCCTGGACGAGCTGTGGCACACCAACGACAACCAGATCGCTAT
CTTCAACCGGCTGAAGCTGGTGCCCAAGAAGGTGGACCTGTCCCAGCAGA
AAGAGATCCCCACCACCCTGGTGGACGACTTCATCCTGAGCCCCGTCGTG
AAGAGAAGCTTCATCCAGAGCATCAAAGTGATCAACGCCATCATCAAGAA
GTACGGCCTGCCCAACGACATCATTATCGAGCTGGCCCGCGAGAAGAACT
CCAAGGACGCCCAGAAAATGATCAACGAGATGCAGAAGCGGAACGCCGCC
ACCAACGAGCGGATCGAGGAAATCATCCGGACCACCGGCAAAGAGAACGC
CAAGTACCTGATCGAGAAGATCAAGCTGCACGACATGCAGGAAGGCAAGT
GCCTGTACAGCCTGGAAGCCATCCCTCTGGAAGATCTGCTGAACAACCCC
TTCAACTATGAGGTGGACCACATCATCCCCAGAAGCGTGTCCTTCGACAA
CAGCTTCAACAACAAGGTGCTCGTGAAGCAGGAAGAAAACAGCAAGAAGG
GCAACCGGACCCCATTCCAGTACCTGAGCAGCAGCGACAGCAAGATCAGC
TACGAAACCTTCAAGAAGCACATCCTGAATCTGGCCAAGGGCAAGGGCAG
AATCAGCAAGACCAAGAAAGAGTATCTGCTGGAAGAACGGGACATCAACA
GGTTCTCCGTGCAGAAAGACTTCATCAACCGGAACCTGGTGGATACCAGA
TACGCCACCGCCGCCCTGATGAACCTGCTGCGGAGCTACTTCAGAGTGAA
CAACCTGGACGTGAAAGTGAAGTCCATCAATGGCGGCTTCACCAGCTTTC
TGCGGCGGAAGTGGAAGTTTAAGAAAGAGCGGAACAAGGGGTACAAGCAC
CACGCCGAGGACGCCCTGATCATTGCCAACGCCGATTTCATCTTCAAAGA
GTGGAAGAAACTGGACAAGGCCAAAAAAGTGATGGAAAACCAGATGTTCG
AGGAAAAGCAGGCCGAGAGCATGCCCGAGATCGAAACCGAGCAGGAGTAC
AAAGAGATCTTCATCACCCCCCACCAGATCAAGCACATTAAGGACTTCAA
GGACTACAAGTACAGCCACCGGGTGGACAAGAAGCCTAATAGAGAGCTGA
TTAACGACACCCTGTACTCCACCCGGAAGGACGACAAGGGCAACACCCTG
ATCGTGAACAATCTGAACGGCCTGTACGACAAGGACAATGACAAGCTGAA
AAAGCTGATCAACAAGAGCCCCGAAAAGCTGCTGATGTACCACCACGACC
CCCAGACCTACCAGAAACTGAAGCTGATTATGGAACAGTACGGCGACGAG
AAGAATCCCCTGTACAAGTACTACGAGGAAACCGGGAACTACCTGACCAA
GTACTCCAAAAAGGACAACGGCCCCGTGATCAAGAAGATTAAGTATTACG
GCAACAAACTGAACGCCCATCTGGACATCACCGACGACTACCCCAACAGC
AGAAACAAGGTCGTGAAGCTGTCCCTGAAGAGCTTCCGCTTCGACATCTA
CAAGTGCGAGCAGGGCTACAAGATGGTGAGCATCGGCTACCTGGACGTGC
TGAAGAAGGACAACTACTACTACATCCCCAAGGACAAGTACGAGGCCGAG
AAGCAGAAGAAGAAGATAAAGGAGAGCGACCTGTTCGTGGGCAGCTTCTA
CTACAACGACCTGATAATGTACGAGGACGAGCTGTTCCGCGTGATAGGCG
TGAACAGCGACATCAACAACCTGGTGGAGCTGAACATGGTGGACATCACC
TACAAGGACTTCTGCGAGGTGAACAACGTGACCGGCGAGAAGCGCATCAA
GAAGACCATCGGCAAGCGCGTGGTGCTGATAGAGAAGTACACCACCGACA
TCCTGGGCAACCTGTACAAGACCCCCCTGCCCAAGAAGCCCCAGCTGATA
TTCAAGCGCGGCGAGCTG;
the humanized Sa-SepCas9 gene sequence:
SEQ ID NO: 28
ATGAAGCGGAACTACATCCTGGGCCTGGACATCGGCATCACCAGCGTGGG
CTACGGCATCATCGACTACGAGACACGGGACGTGATCGATGCCGGCGTGC
GGCTGTTCAAAGAGGCCAACGTGGAAAACAACGAGGGCAGGCGGAGCAAG
AGAGGCGCCAGAAGGCTGAAGCGGCGGAGGCGGCATAGAATCCAGAGAGT
GAAGAAGCTGCTGTTCGACTACAACCTGCTGACCGACCACAGCGAGCTGA
GCGGCATCAACCCCTACGAGGCCAGAGTGAAGGGCCTGAGCCAGAAGCTG
AGCGAGGAAGAGTTCTCTGCCGCCCTGCTGCACCTGGCCAAGAGAAGAGG
CGTGCACAACGTGAACGAGGTGGAAGAGGACACCGGCAACGAGCTGTCCA
CCAAAGAGCAGATCAGCCGGAACAGCAAGGCCCTGGAAGAGAAATACGTG
GCCGAACTGCAGCTGGAACGGCTGAAGAAAGACGGCGAAGTGCGGGGCAG
CATCAACAGATTCAAGACCAGCGACTACGTGAAAGAAGCCAAACAGCTGC
TGAAGGTGCAGAAGGCCTACCACCAGCTGGACCAGAGCTTCATCGACACC
TACATCGACCTGCTGGAAACCCGGCGGACCTACTATGAGGGACCTGGCGA
GGGCAGCCCCTTCGGCTGGAAGGACATCAAAGAATGGTACGAGATGCTGA
TGGGCCACTGCACCTACTTCCCCGAGGAACTGCGGAGCGTGAAGTACGCC
TACAACGCCGACCTGTACAACGCCCTGAACGACCTGAACAATCTCGTGAT
CACCAGGGACGAGAACGAGAAGCTGGAATATTACGAGAAGTTCCAGATCA
TCGAGAACGTGTTCAAGCAGAAGAAGAAGCCCACCCTGAAGCAGATCGCC
AAAGAAATCCTCGTGAACGAAGAGGATATTAAGGGCTACAGAGTGACCAG
CACCGGCAAGCCCGAGTTCACCAACCTGAAGGTGTACCACGACATCAAGG
ACATTACCGCCCGGAAAGAGATTATTGAGAACGCCGAGCTGCTGGATCAG
ATTGCCAAGATCCTGACCATCTACCAGAGCAGCGAGGACATCCAGGAAGA
ACTGACCAATCTGAACTCCGAGCTGACCCAGGAAGAGATCGAGCAGATCT
CTAATCTGAAGGGCTATACCGGCACCCACAACCTGAGCCTGAAGGCCATC
AACCTGATCCTGGACGAGCTGTGGCACACCAACGACAACCAGATCGCTAT
CTTCAACCGGCTGAAGCTGGTGCCCAAGAAGGTGGACCTGTCCCAGCAGA
AAGAGATCCCCACCACCCTGGTGGACGACTTCATCCTGAGCCCCGTCGTG
AAGAGAAGCTTCATCCAGAGCATCAAAGTGATCAACGCCATCATCAAGAA
GTACGGCCTGCCCAACGACATCATTATCGAGCTGGCCCGCGAGAAGAACT
CCAAGGACGCCCAGAAAATGATCAACGAGATGCAGAAGCGGAACGCCGCC
ACCAACGAGCGGATCGAGGAAATCATCCGGACCACCGGCAAAGAGAACGC
CAAGTACCTGATCGAGAAGATCAAGCTGCACGACATGCAGGAAGGCAAGT
GCCTGTACAGCCTGGAAGCCATCCCTCTGGAAGATCTGCTGAACAACCCC
TTCAACTATGAGGTGGACCACATCATCCCCAGAAGCGTGTCCTTCGACAA
CAGCTTCAACAACAAGGTGCTCGTGAAGCAGGAAGAAAACAGCAAGAAGG
GCAACCGGACCCCATTCCAGTACCTGAGCAGCAGCGACAGCAAGATCAGC
TACGAAACCTTCAAGAAGCACATCCTGAATCTGGCCAAGGGCAAGGGCAG
AATCAGCAAGACCAAGAAAGAGTATCTGCTGGAAGAACGGGACATCAACA
GGTTCTCCGTGCAGAAAGACTTCATCAACCGGAACCTGGTGGATACCAGA
TACGCCACCGCCGCCCTGATGAACCTGCTGCGGAGCTACTTCAGAGTGAA
CAACCTGGACGTGAAAGTGAAGTCCATCAATGGCGGCTTCACCAGCTTTC
TGCGGCGGAAGTGGAAGTTTAAGAAAGAGCGGAACAAGGGGTACAAGCAC
CACGCCGAGGACGCCCTGATCATTGCCAACGCCGATTTCATCTTCAAAGA
GTGGAAGAAACTGGACAAGGCCAAAAAAGTGATGGAAAACCAGATGTTCG
AGGAAAAGCAGGCCGAGAGCATGCCCGAGATCGAAACCGAGCAGGAGTAC
AAAGAGATCTTCATCACCCCCCACCAGATCAAGCACATTAAGGACTTCAA
GGACTACAAGTACAGCCACCGGGTGGACAAGAAGCCTAATAGAGAGCTGA
TTAACGACACCCTGTACTCCACCCGGAAGGACGACAAGGGCAACACCCTG
ATCGTGAACAATCTGAACGGCCTGTACGACAAGGACAATGACAAGCTGAA
AAAGCTGATCAACAAGAGCCCCGAAAAGCTGCTGATGTACCACCACGACC
CCCAGACCTACCAGAAACTGAAGCTGATTATGGAACAGTACGGCGACGAG
AAGAATCCCCTGTACAAGTACTACGAGGAAACCGGGAACTACCTGACCAA
GTACTCCAAAAAGGACAACGGCCCCGTGATCAAGAAGATTAAGTATTACG
GCAACAAACTGAACGCCCATCTGGACATCACCGACGACTACCCCAACAGC
AGAAACAAGGTCGTGAAGCTGTCCCTGAAGAACTACCGCTTCGACGTGTA
CCTGACCGAGAAGGGCTACAAGTTCGTGACCATCGCCTACCTGAACGTGT
TCAAGAAGGACAACTACTACTACATCCCCAAGGACATGTACCAGGAGCTG
AAGGCCAAGAAGAAGATAAAGGACACCGACCAGTTCATCGCCAGCTTCTA
CAAGAACGACCTGATAAAGCTGAACGGCGACCTGTACAAGATAATCGGCG
TGAACAGCGACGACCGCAACATCATCGAGCTGGACTACTACGACATCAAG
TACAAGGACTACTGCGAGATAAACAACATCAAGGGCGAGCCCCGCATCAA
GAAGACCATCGGCAAGAAGACCGAGAGCATCGAGAAGCTGACCACCGACG
TGCTGGGCAACCTGTACCTGCACAGCACCGAGAAGGCCCCCCAGCTGATA
TTCAAGCGCGGCCTG;
the humanized Sa-SeqCas9 gene sequence:
SEQ ID NO: 29
ATGAAGCGGAACTACATCCTGGGCCTGGACATCGGCATCACCAGCGTGGG
CTACGGCATCATCGACTACGAGACACGGGACGTGATCGATGCCGGCGTGC
GGCTGTTCAAAGAGGCCAACGTGGAAAACAACGAGGGCAGGCGGAGCAAG
AGAGGCGCCAGAAGGCTGAAGCGGCGGAGGCGGCATAGAATCCAGAGAGT
GAAGAAGCTGCTGTTCGACTACAACCTGCTGACCGACCACAGCGAGCTGA
GCGGCATCAACCCCTACGAGGCCAGAGTGAAGGGCCTGAGCCAGAAGCTG
AGCGAGGAAGAGTTCTCTGCCGCCCTGCTGCACCTGGCCAAGAGAAGAGG
CGTGCACAACGTGAACGAGGTGGAAGAGGACACCGGCAACGAGCTGTCCA
CCAAAGAGCAGATCAGCCGGAACAGCAAGGCCCTGGAAGAGAAATACGTG
GCCGAACTGCAGCTGGAACGGCTGAAGAAAGACGGCGAAGTGCGGGGCAG
CATCAACAGATTCAAGACCAGCGACTACGTGAAAGAAGCCAAACAGCTGC
TGAAGGTGCAGAAGGCCTACCACCAGCTGGACCAGAGCTTCATCGACACC
TACATCGACCTGCTGGAAACCCGGCGGACCTACTATGAGGGACCTGGCGA
GGGCAGCCCCTTCGGCTGGAAGGACATCAAAGAATGGTACGAGATGCTGA
TGGGCCACTGCACCTACTTCCCCGAGGAACTGCGGAGCGTGAAGTACGCC
TACAACGCCGACCTGTACAACGCCCTGAACGACCTGAACAATCTCGTGAT
CACCAGGGACGAGAACGAGAAGCTGGAATATTACGAGAAGTTCCAGATCA
TCGAGAACGTGTTCAAGCAGAAGAAGAAGCCCACCCTGAAGCAGATCGCC
AAAGAAATCCTCGTGAACGAAGAGGATATTAAGGGCTACAGAGTGACCAG
CACCGGCAAGCCCGAGTTCACCAACCTGAAGGTGTACCACGACATCAAGG
ACATTACCGCCCGGAAAGAGATTATTGAGAACGCCGAGCTGCTGGATCAG
ATTGCCAAGATCCTGACCATCTACCAGAGCAGCGAGGACATCCAGGAAGA
ACTGACCAATCTGAACTCCGAGCTGACCCAGGAAGAGATCGAGCAGATCT
CTAATCTGAAGGGCTATACCGGCACCCACAACCTGAGCCTGAAGGCCATC
AACCTGATCCTGGACGAGCTGTGGCACACCAACGACAACCAGATCGCTAT
CTTCAACCGGCTGAAGCTGGTGCCCAAGAAGGTGGACCTGTCCCAGCAGA
AAGAGATCCCCACCACCCTGGTGGACGACTTCATCCTGAGCCCCGTCGTG
AAGAGAAGCTTCATCCAGAGCATCAAAGTGATCAACGCCATCATCAAGAA
GTACGGCCTGCCCAACGACATCATTATCGAGCTGGCCCGCGAGAAGAACT
CCAAGGACGCCCAGAAAATGATCAACGAGATGCAGAAGCGGAACGCCGCC
ACCAACGAGCGGATCGAGGAAATCATCCGGACCACCGGCAAAGAGAACGC
CAAGTACCTGATCGAGAAGATCAAGCTGCACGACATGCAGGAAGGCAAGT
GCCTGTACAGCCTGGAAGCCATCCCTCTGGAAGATCTGCTGAACAACCCC
TTCAACTATGAGGTGGACCACATCATCCCCAGAAGCGTGTCCTTCGACAA
CAGCTTCAACAACAAGGTGCTCGTGAAGCAGGAAGAAAACAGCAAGAAGG
GCAACCGGACCCCATTCCAGTACCTGAGCAGCAGCGACAGCAAGATCAGC
TACGAAACCTTCAAGAAGCACATCCTGAATCTGGCCAAGGGCAAGGGCAG
AATCAGCAAGACCAAGAAAGAGTATCTGCTGGAAGAACGGGACATCAACA
GGTTCTCCGTGCAGAAAGACTTCATCAACCGGAACCTGGTGGATACCAGA
TACGCCACCGCCGCCCTGATGAACCTGCTGCGGAGCTACTTCAGAGTGAA
CAACCTGGACGTGAAAGTGAAGTCCATCAATGGCGGCTTCACCAGCTTTC
TGCGGCGGAAGTGGAAGTTTAAGAAAGAGCGGAACAAGGGGTACAAGCAC
CACGCCGAGGACGCCCTGATCATTGCCAACGCCGATTTCATCTTCAAAGA
GTGGAAGAAACTGGACAAGGCCAAAAAAGTGATGGAAAACCAGATGTTCG
AGGAAAAGCAGGCCGAGAGCATGCCCGAGATCGAAACCGAGCAGGAGTAC
AAAGAGATCTTCATCACCCCCCACCAGATCAAGCACATTAAGGACTTCAA
GGACTACAAGTACAGCCACCGGGTGGACAAGAAGCCTAATAGAGAGCTGA
TTAACGACACCCTGTACTCCACCCGGAAGGACGACAAGGGCAACACCCTG
ATCGTGAACAATCTGAACGGCCTGTACGACAAGGACAATGACAAGCTGAA
AAAGCTGATCAACAAGAGCCCCGAAAAGCTGCTGATGTACCACCACGACC
CCCAGACCTACCAGAAACTGAAGCTGATTATGGAACAGTACGGCGACGAG
AAGAATCCCCTGTACAAGTACTACGAGGAAACCGGGAACTACCTGACCAA
GTACTCCAAAAAGGACAACGGCCCCGTGATCAAGAAGATTAAGTATTACG
GCAACAAACTGAACGCCCATCTGGACATCACCGACGACTACCCCAACAGC
AGAAACAAGGTCGTGAAGCTGTCCCTGAAGCCCTACCGCTTCGACGTGTA
CCTGGACAACGGCGTGTACAAGTTCGTGACCGTGAAGAACCTGAACGTGA
TAAAGAAGGAGAACTACTACGAGGTGAACAGCAAGTGCTACGAGAAGGCC
AAGAAGCTGAAGAAGATAAGCGACCAGGCCGAGTTCATCGCCAGCTTCTA
CAACAACGACCTGATAAAGATAGACGGCGAGCTGTACCGCGTGATAGGCG
TGAACACCGACCTGATAAACCGCATCGAGGTGAACATGGTGGACATCACC
TACCGCGAGTACCTGGAGAACATGAACGACAAGCGCAGCCCCCGCATCTT
CAAGACCATCGCCAGCAAGACCCAGAGCATCAAGAAGTACAGCACCGACA
TCCTGGGCACCCTGTACGAGGTGAACAGCAAGAAGCACCCCCAGATGATA
ATGAAGGGC;
the humanized Sa-ShaCas9 gene sequence:
SEQ ID NO: 30
ATGAAGCGGAACTACATCCTGGGCCTGGACATCGGCATCACCAGCGTGGG
CTACGGCATCATCGACTACGAGACACGGGACGTGATCGATGCCGGCGTGC
GGCTGTTCAAAGAGGCCAACGTGGAAAACAACGAGGGCAGGCGGAGCAAG
AGAGGCGCCAGAAGGCTGAAGCGGCGGAGGCGGCATAGAATCCAGAGAGT
GAAGAAGCTGCTGTTCGACTACAACCTGCTGACCGACCACAGCGAGCTGA
GCGGCATCAACCCCTACGAGGCCAGAGTGAAGGGCCTGAGCCAGAAGCTG
AGCGAGGAAGAGTTCTCTGCCGCCCTGCTGCACCTGGCCAAGAGAAGAGG
CGTGCACAACGTGAACGAGGTGGAAGAGGACACCGGCAACGAGCTGTCCA
CCAAAGAGCAGATCAGCCGGAACAGCAAGGCCCTGGAAGAGAAATACGTG
GCCGAACTGCAGCTGGAACGGCTGAAGAAAGACGGCGAAGTGCGGGGCAG
CATCAACAGATTCAAGACCAGCGACTACGTGAAAGAAGCCAAACAGCTGC
TGAAGGTGCAGAAGGCCTACCACCAGCTGGACCAGAGCTTCATCGACACC
TACATCGACCTGCTGGAAACCCGGCGGACCTACTATGAGGGACCTGGCGA
GGGCAGCCCCTTCGGCTGGAAGGACATCAAAGAATGGTACGAGATGCTGA
TGGGCCACTGCACCTACTTCCCCGAGGAACTGCGGAGCGTGAAGTACGCC
TACAACGCCGACCTGTACAACGCCCTGAACGACCTGAACAATCTCGTGAT
CACCAGGGACGAGAACGAGAAGCTGGAATATTACGAGAAGTTCCAGATCA
TCGAGAACGTGTTCAAGCAGAAGAAGAAGCCCACCCTGAAGCAGATCGCC
AAAGAAATCCTCGTGAACGAAGAGGATATTAAGGGCTACAGAGTGACCAG
CACCGGCAAGCCCGAGTTCACCAACCTGAAGGTGTACCACGACATCAAGG
ACATTACCGCCCGGAAAGAGATTATTGAGAACGCCGAGCTGCTGGATCAG
ATTGCCAAGATCCTGACCATCTACCAGAGCAGCGAGGACATCCAGGAAGA
ACTGACCAATCTGAACTCCGAGCTGACCCAGGAAGAGATCGAGCAGATCT
CTAATCTGAAGGGCTATACCGGCACCCACAACCTGAGCCTGAAGGCCATC
AACCTGATCCTGGACGAGCTGTGGCACACCAACGACAACCAGATCGCTAT
CTTCAACCGGCTGAAGCTGGTGCCCAAGAAGGTGGACCTGTCCCAGCAGA
AAGAGATCCCCACCACCCTGGTGGACGACTTCATCCTGAGCCCCGTCGTG
AAGAGAAGCTTCATCCAGAGCATCAAAGTGATCAACGCCATCATCAAGAA
GTACGGCCTGCCCAACGACATCATTATCGAGCTGGCCCGCGAGAAGAACT
CCAAGGACGCCCAGAAAATGATCAACGAGATGCAGAAGCGGAACGCCGCC
ACCAACGAGCGGATCGAGGAAATCATCCGGACCACCGGCAAAGAGAACGC
CAAGTACCTGATCGAGAAGATCAAGCTGCACGACATGCAGGAAGGCAAGT
GCCTGTACAGCCTGGAAGCCATCCCTCTGGAAGATCTGCTGAACAACCCC
TTCAACTATGAGGTGGACCACATCATCCCCAGAAGCGTGTCCTTCGACAA
CAGCTTCAACAACAAGGTGCTCGTGAAGCAGGAAGAAAACAGCAAGAAGG
GCAACCGGACCCCATTCCAGTACCTGAGCAGCAGCGACAGCAAGATCAGC
TACGAAACCTTCAAGAAGCACATCCTGAATCTGGCCAAGGGCAAGGGCAG
AATCAGCAAGACCAAGAAAGAGTATCTGCTGGAAGAACGGGACATCAACA
GGTTCTCCGTGCAGAAAGACTTCATCAACCGGAACCTGGTGGATACCAGA
TACGCCACCGCCGCCCTGATGAACCTGCTGCGGAGCTACTTCAGAGTGAA
CAACCTGGACGTGAAAGTGAAGTCCATCAATGGCGGCTTCACCAGCTTTC
TGCGGCGGAAGTGGAAGTTTAAGAAAGAGCGGAACAAGGGGTACAAGCAC
CACGCCGAGGACGCCCTGATCATTGCCAACGCCGATTTCATCTTCAAAGA
GTGGAAGAAACTGGACAAGGCCAAAAAAGTGATGGAAAACCAGATGTTCG
AGGAAAAGCAGGCCGAGAGCATGCCCGAGATCGAAACCGAGCAGGAGTAC
AAAGAGATCTTCATCACCCCCCACCAGATCAAGCACATTAAGGACTTCAA
GGACTACAAGTACAGCCACCGGGTGGACAAGAAGCCTAATAGAGAGCTGA
TTAACGACACCCTGTACTCCACCCGGAAGGACGACAAGGGCAACACCCTG
ATCGTGAACAATCTGAACGGCCTGTACGACAAGGACAATGACAAGCTGAA
AAAGCTGATCAACAAGAGCCCCGAAAAGCTGCTGATGTACCACCACGACC
CCCAGACCTACCAGAAACTGAAGCTGATTATGGAACAGTACGGCGACGAG
AAGAATCCCCTGTACAAGTACTACGAGGAAACCGGGAACTACCTGACCAA
GTACTCCAAAAAGGACAACGGCCCCGTGATCAAGAAGATTAAGTATTACG
GCAACAAACTGAACGCCCATCTGGACATCACCGACGACTACCCCAACAGC
AGAAACAAGGTCGTGAAGCTGTCCCTGAAGAGCTACCGCTTCGACGTGTA
CCTGACCGACAAGGGCTACAAGTTCGTGAGCATCACCTACCTGGACGTGC
TGAAGAAGGAGAACTACTACTACATCAGCGAGGCCAAGTACGACAAGCTG
AAGCTGAACAAGGGCATCGACGACAAGGCCAAGTTCATCGGCAGCTTCTA
CTACAACGACCTGATAGAGCTGGACGGCGAGGTGTACACCGTGATAGGCG
TGAACAACGACAAGAACAACGTGATAGAGCTGAACCTGCCCGAGATACGC
TACAAGGAGTACTGCGAGATAAACAACATCAAGGGCAGCGGCAGGCTGCG
CATCACCATCGGCAAGAAGGTGAACAGCATCCGCAAGCTGAGCACCGACG
TGCTGGGCAACCGCTACTACCAGAGCTTCGCCAAGAAGCCCCAGCTGGTG
TTCAAGAAGGGCATC;
the humanized Sa-SlugCas9 gene sequence:
SEQ ID NO: 31
ATGAAGCGGAACTACATCCTGGGCCTGGACATCGGCATCACCAGCGTGGG
CTACGGCATCATCGACTACGAGACACGGGACGTGATCGATGCCGGCGTGC
GGCTGTTCAAAGAGGCCAACGTGGAAAACAACGAGGGCAGGCGGAGCAAG
AGAGGCGCCAGAAGGCTGAAGCGGCGGAGGCGGCATAGAATCCAGAGAGT
GAAGAAGCTGCTGTTCGACTACAACCTGCTGACCGACCACAGCGAGCTGA
GCGGCATCAACCCCTACGAGGCCAGAGTGAAGGGCCTGAGCCAGAAGCTG
AGCGAGGAAGAGTTCTCTGCCGCCCTGCTGCACCTGGCCAAGAGAAGAGG
CGTGCACAACGTGAACGAGGTGGAAGAGGACACCGGCAACGAGCTGTCCA
CCAAAGAGCAGATCAGCCGGAACAGCAAGGCCCTGGAAGAGAAATACGTG
GCCGAACTGCAGCTGGAACGGCTGAAGAAAGACGGCGAAGTGCGGGGCAG
CATCAACAGATTCAAGACCAGCGACTACGTGAAAGAAGCCAAACAGCTGC
TGAAGGTGCAGAAGGCCTACCACCAGCTGGACCAGAGCTTCATCGACACC
TACATCGACCTGCTGGAAACCCGGCGGACCTACTATGAGGGACCTGGCGA
GGGCAGCCCCTTCGGCTGGAAGGACATCAAAGAATGGTACGAGATGCTGA
TGGGCCACTGCACCTACTTCCCCGAGGAACTGCGGAGCGTGAAGTACGCC
TACAACGCCGACCTGTACAACGCCCTGAACGACCTGAACAATCTCGTGAT
CACCAGGGACGAGAACGAGAAGCTGGAATATTACGAGAAGTTCCAGATCA
TCGAGAACGTGTTCAAGCAGAAGAAGAAGCCCACCCTGAAGCAGATCGCC
AAAGAAATCCTCGTGAACGAAGAGGATATTAAGGGCTACAGAGTGACCAG
CACCGGCAAGCCCGAGTTCACCAACCTGAAGGTGTACCACGACATCAAGG
ACATTACCGCCCGGAAAGAGATTATTGAGAACGCCGAGCTGCTGGATCAG
ATTGCCAAGATCCTGACCATCTACCAGAGCAGCGAGGACATCCAGGAAGA
ACTGACCAATCTGAACTCCGAGCTGACCCAGGAAGAGATCGAGCAGATCT
CTAATCTGAAGGGCTATACCGGCACCCACAACCTGAGCCTGAAGGCCATC
AACCTGATCCTGGACGAGCTGTGGCACACCAACGACAACCAGATCGCTAT
CTTCAACCGGCTGAAGCTGGTGCCCAAGAAGGTGGACCTGTCCCAGCAGA
AAGAGATCCCCACCACCCTGGTGGACGACTTCATCCTGAGCCCCGTCGTG
AAGAGAAGCTTCATCCAGAGCATCAAAGTGATCAACGCCATCATCAAGAA
GTACGGCCTGCCCAACGACATCATTATCGAGCTGGCCCGCGAGAAGAACT
CCAAGGACGCCCAGAAAATGATCAACGAGATGCAGAAGCGGAACGCCGCC
ACCAACGAGCGGATCGAGGAAATCATCCGGACCACCGGCAAAGAGAACGC
CAAGTACCTGATCGAGAAGATCAAGCTGCACGACATGCAGGAAGGCAAGT
GCCTGTACAGCCTGGAAGCCATCCCTCTGGAAGATCTGCTGAACAACCCC
TTCAACTATGAGGTGGACCACATCATCCCCAGAAGCGTGTCCTTCGACAA
CAGCTTCAACAACAAGGTGCTCGTGAAGCAGGAAGAAAACAGCAAGAAGG
GCAACCGGACCCCATTCCAGTACCTGAGCAGCAGCGACAGCAAGATCAGC
TACGAAACCTTCAAGAAGCACATCCTGAATCTGGCCAAGGGCAAGGGCAG
AATCAGCAAGACCAAGAAAGAGTATCTGCTGGAAGAACGGGACATCAACA
GGTTCTCCGTGCAGAAAGACTTCATCAACCGGAACCTGGTGGATACCAGA
TACGCCACCGCCGCCCTGATGAACCTGCTGCGGAGCTACTTCAGAGTGAA
CAACCTGGACGTGAAAGTGAAGTCCATCAATGGCGGCTTCACCAGCTTTC
TGCGGCGGAAGTGGAAGTTTAAGAAAGAGCGGAACAAGGGGTACAAGCAC
CACGCCGAGGACGCCCTGATCATTGCCAACGCCGATTTCATCTTCAAAGA
GTGGAAGAAACTGGACAAGGCCAAAAAAGTGATGGAAAACCAGATGTTCG
AGGAAAAGCAGGCCGAGAGCATGCCCGAGATCGAAACCGAGCAGGAGTAC
AAAGAGATCTTCATCACCCCCCACCAGATCAAGCACATTAAGGACTTCAA
GGACTACAAGTACAGCCACCGGGTGGACAAGAAGCCTAATAGAGAGCTGA
TTAACGACACCCTGTACTCCACCCGGAAGGACGACAAGGGCAACACCCTG
ATCGTGAACAATCTGAACGGCCTGTACGACAAGGACAATGACAAGCTGAA
AAAGCTGATCAACAAGAGCCCCGAAAAGCTGCTGATGTACCACCACGACC
CCCAGACCTACCAGAAACTGAAGCTGATTATGGAACAGTACGGCGACGAG
AAGAATCCCCTGTACAAGTACTACGAGGAAACCGGGAACTACCTGACCAA
GTACTCCAAAAAGGACAACGGCCCCGTGATCAAGAAGATTAAGTATTACG
GCAACAAACTGAACGCCCATCTGGACATCACCGACGACTACCCCAACAGC
AGAAACAAGGTCGTGAAGCTGTCCCTGAAGCCCTACCGCTTCGACGTGTA
CCTGACCGACAAGGGCTACAAGTTCATCACCATCAGCTACCTGGACGTGC
TGAAGAAGGACAACTACTACTACATCCCCGAGCAGAAGTACGACAAGCTG
AAGCTGGGCAAGGCCATCGACAAGAACGCCAAGTTCATCGCCAGCTTCTA
CAAGAACGACCTGATAAAGCTGGACGGCGAGATATACAAGATAATCGGCG
TGAACAGCGACACCCGCAACATGATAGAGCTGGACCTGCCCGACATCCGC
TACAAGGAGTACTGCGAGCTGAACAACATCAAGGGCGAGCCCCGCATCAA
GAAGACCATCGGCAAGAAGGTGAACAGCATCGAGAAGCTGACCACCGACG
TGCTGGGCAACGTGTTCACCAACACCCAGTACACCAAGCCCCAGCTGCTG
TTCAAGCGCGGCAAC;
the humanized Sa-SlutCas9 gene sequence:
SEQ ID NO: 32
ATGAAGCGGAACTACATCCTGGGCCTGGACATCGGCATCACCAGCGTGGG
CTACGGCATCATCGACTACGAGACACGGGACGTGATCGATGCCGGCGTGC
GGCTGTTCAAAGAGGCCAACGTGGAAAACAACGAGGGCAGGCGGAGCAAG
AGAGGCGCCAGAAGGCTGAAGCGGCGGAGGCGGCATAGAATCCAGAGAGT
GAAGAAGCTGCTGTTCGACTACAACCTGCTGACCGACCACAGCGAGCTGA
GCGGCATCAACCCCTACGAGGCCAGAGTGAAGGGCCTGAGCCAGAAGCTG
AGCGAGGAAGAGTTCTCTGCCGCCCTGCTGCACCTGGCCAAGAGAAGAGG
CGTGCACAACGTGAACGAGGTGGAAGAGGACACCGGCAACGAGCTGTCCA
CCAAAGAGCAGATCAGCCGGAACAGCAAGGCCCTGGAAGAGAAATACGTG
GCCGAACTGCAGCTGGAACGGCTGAAGAAAGACGGCGAAGTGCGGGGCAG
CATCAACAGATTCAAGACCAGCGACTACGTGAAAGAAGCCAAACAGCTGC
TGAAGGTGCAGAAGGCCTACCACCAGCTGGACCAGAGCTTCATCGACACC
TACATCGACCTGCTGGAAACCCGGCGGACCTACTATGAGGGACCTGGCGA
GGGCAGCCCCTTCGGCTGGAAGGACATCAAAGAATGGTACGAGATGCTGA
TGGGCCACTGCACCTACTTCCCCGAGGAACTGCGGAGCGTGAAGTACGCC
TACAACGCCGACCTGTACAACGCCCTGAACGACCTGAACAATCTCGTGAT
CACCAGGGACGAGAACGAGAAGCTGGAATATTACGAGAAGTTCCAGATCA
TCGAGAACGTGTTCAAGCAGAAGAAGAAGCCCACCCTGAAGCAGATCGCC
AAAGAAATCCTCGTGAACGAAGAGGATATTAAGGGCTACAGAGTGACCAG
CACCGGCAAGCCCGAGTTCACCAACCTGAAGGTGTACCACGACATCAAGG
ACATTACCGCCCGGAAAGAGATTATTGAGAACGCCGAGCTGCTGGATCAG
ATTGCCAAGATCCTGACCATCTACCAGAGCAGCGAGGACATCCAGGAAGA
ACTGACCAATCTGAACTCCGAGCTGACCCAGGAAGAGATCGAGCAGATCT
CTAATCTGAAGGGCTATACCGGCACCCACAACCTGAGCCTGAAGGCCATC
AACCTGATCCTGGACGAGCTGTGGCACACCAACGACAACCAGATCGCTAT
CTTCAACCGGCTGAAGCTGGTGCCCAAGAAGGTGGACCTGTCCCAGCAGA
AAGAGATCCCCACCACCCTGGTGGACGACTTCATCCTGAGCCCCGTCGTG
AAGAGAAGCTTCATCCAGAGCATCAAAGTGATCAACGCCATCATCAAGAA
GTACGGCCTGCCCAACGACATCATTATCGAGCTGGCCCGCGAGAAGAACT
CCAAGGACGCCCAGAAAATGATCAACGAGATGCAGAAGCGGAACGCCGCC
ACCAACGAGCGGATCGAGGAAATCATCCGGACCACCGGCAAAGAGAACGC
CAAGTACCTGATCGAGAAGATCAAGCTGCACGACATGCAGGAAGGCAAGT
GCCTGTACAGCCTGGAAGCCATCCCTCTGGAAGATCTGCTGAACAACCCC
TTCAACTATGAGGTGGACCACATCATCCCCAGAAGCGTGTCCTTCGACAA
CAGCTTCAACAACAAGGTGCTCGTGAAGCAGGAAGAAAACAGCAAGAAGG
GCAACCGGACCCCATTCCAGTACCTGAGCAGCAGCGACAGCAAGATCAGC
TACGAAACCTTCAAGAAGCACATCCTGAATCTGGCCAAGGGCAAGGGCAG
AATCAGCAAGACCAAGAAAGAGTATCTGCTGGAAGAACGGGACATCAACA
GGTTCTCCGTGCAGAAAGACTTCATCAACCGGAACCTGGTGGATACCAGA
TACGCCACCGCCGCCCTGATGAACCTGCTGCGGAGCTACTTCAGAGTGAA
CAACCTGGACGTGAAAGTGAAGTCCATCAATGGCGGCTTCACCAGCTTTC
TGCGGCGGAAGTGGAAGTTTAAGAAAGAGCGGAACAAGGGGTACAAGCAC
CACGCCGAGGACGCCCTGATCATTGCCAACGCCGATTTCATCTTCAAAGA
GTGGAAGAAACTGGACAAGGCCAAAAAAGTGATGGAAAACCAGATGTTCG
AGGAAAAGCAGGCCGAGAGCATGCCCGAGATCGAAACCGAGCAGGAGTAC
AAAGAGATCTTCATCACCCCCCACCAGATCAAGCACATTAAGGACTTCAA
GGACTACAAGTACAGCCACCGGGTGGACAAGAAGCCTAATAGAGAGCTGA
TTAACGACACCCTGTACTCCACCCGGAAGGACGACAAGGGCAACACCCTG
ATCGTGAACAATCTGAACGGCCTGTACGACAAGGACAATGACAAGCTGAA
AAAGCTGATCAACAAGAGCCCCGAAAAGCTGCTGATGTACCACCACGACC
CCCAGACCTACCAGAAACTGAAGCTGATTATGGAACAGTACGGCGACGAG
AAGAATCCCCTGTACAAGTACTACGAGGAAACCGGGAACTACCTGACCAA
GTACTCCAAAAAGGACAACGGCCCCGTGATCAAGAAGATTAAGTATTACG
GCAACAAACTGAACGCCCATCTGGACATCACCGACGACTACCCCAACAGC
AGAAACAAGGTCGTGAAGCTGTCCCTGAAGAGCTTCCGCTTCGACATCTA
CCACACCGACAAGGGCTACAAGATGGTGCCCATCACCTACCTGGACGTGC
AGAAGAAGGAGAAGTACTACTACATCCCCACCGAGAAGTACGAGGCCCTG
AAGCAGGAGAAGGGCATCAACCAGAACGCCCAGTTCATCGGCAGCTTCTA
CTACAACGACCTGATAGAGTTCGACGGCGAGCTGTACCGCGTGATAGGCA
TCAACAACGGCGACAAGAACCTGGTGGAGCTGGACATGGTGGACATCCGC
TACAAGGAGTACTGCGAGCTGAACAGCATCACCACCACCCCCCGCATCGT
GAAGACCATCAGCCCCAAGACCCAGAGCATCGAGAAGTACACCACCGACA
TCCTGGGCAACCTGTACAAGGCCCAGCCCGGCAAGAAGCCCCAGTTCATC
TTCAACAAGGACGAGGAC;
the humanized SlugCas9-HF gene sequence:
SEQ ID NO: 112
ATGAACCAAAAATTCATACTGGGACTGGACATCGGAATCACCAGCGTGGG
CTACGGCCTGATCGACTACGAGACAAAGAATATCATCGATGCCGGCGTTA
GACTGTTCCCCGAGGCCAACGTGGAAAACAACGAGGGAAGAAGGTCCAAA
CGTGGAAGCAGAAGACTGAAGCGACGCCGCATTCACAGACTTGAACGGGT
GAAGAAGCTGCTCGAGGATTATAATCTGCTGGATCAGTCCCAGATTCCTC
AGTCTACAAACCCCTACGCCATCCGCGTGAAGGGCCTGTCTGAAGCCCTG
AGCAAGGACGAACTCGTGATTGCCCTGCTCCATATCGCCAAGAGAAGAGG
CATCCACAAGATCGACGTGATCGACAGCAACGACGACGTGGGGAACGAGC
TCAGCACCAAGGAACAGCTGAATAAGAACAGCAAGCTGCTGAAAGACAAA
TTTGTGTGCCAGATCCAGCTGGAAAGAATGAATGAGGGCCAGGTGCGGGG
AGAGAAAAACCGGTTCAAGACCGCTGATATCATCAAGGAAATCATCCAGC
TGCTGAATGTGCAGAAGAACTTCCACCAGCTGGACGAGAACTTCATCAAC
AAGTACATCGAACTGGTTGAGATGAGACGGGAATACTTCGAGGGCCCCGG
CAAGGGCAGTCCATATGGCTGGGAAGGCGACCCTAAGGCTTGGTACGAGA
CACTGATGGGCCACTGCACCTACTTCCCAGATGAGCTGgctAGCGTGAAA
TACGCCTACAGTGCCGACCTGTTCAACGCTCTGAACGACCTGAACAACCT
GGTCATCCAAAGAGATGGACTGTCTAAGCTCGAGTATCATGAGAAGTATC
ACATCATCGAGAACGTGTTCAAGCAGAAGAAGAAACCTACACTGAAGCAG
ATCGCCAATGAGATCAATGTCAACCCTGAAGATATCAAGGGCTACAGAAT
CACAAAGTCTGGCAAGCCCCAGTTTACCGAGTTTAAGCTCTACCACGACC
TGAAAAGCGTGCTGTTTGACCAGAGCATCCTGGAGAACGAAGACGTGCTG
GACCAGATCGCTGAGATCCTGACCATCTACCAGGACAAGGATAGCATCAA
ATCTAAGCTGACGGAACTGGACATCCTGCTGAACGAGGAAGATAAGGAAA
ACATCGCCCAGCTGACTGGCTACACCGGGACCCACCGGCTCAGCCTGAAA
TGCATCCGGCTGGTCCTGGAAGAGCAGTGGTATTCTAGCCGGgcTCAGAT
GGAAATCTTCgccCACCTGAACATTAAGCCTAAGAAGATCAACCTGACAG
CCGCCAACAAGATCCCGAAGGCTATGATCGACGAGTTCATCCTGAGCCCT
GTGGTGAAGAGGACCTTCGGCCAGGCCATTAACCTTATTAACAAGATCAT
AGAAAAGTACGGCGTGCCTGAAGATATCATCATCGAGCTGGCCAGAGAAA
ATAATAGCAAGGACAAGCAGAAGTTCATCAATGAGATGCAGAAAAAGAAC
GAGAACACCAGAAAGAGAATTAACGAAATCATCGGCAAGTATGGCAACCA
GAACGCCAAGAGACTGGTCGAGAAGATTAGACTGCACGACGAGCAGGAGG
GCAAGTGCCTGTACTCACTGGAAAGCATCCCTCTGGAGGACCTGCTGAAC
AACCCCAACCACTACGAGGTGGACCACATCATTCCAAGATCTGTGTCCTT
CGACAACTCTTACCACAACAAAGTGCTCGTGAAGCAGAGCGAGAACTCCA
AAAAATCCAACCTGACCCCTTACCAGTACTTTAACAGCGGCAAGTCCAAG
CTCTCTTACAACCAGTTTAAACAACACATCCTGAACCTGAGCAAGTCCCA
GGATAGAATCAGCAAAAAAAAGAAAGAGTATCTGCTGGAAGAACGGGACA
TCAACAAGTTCGAGGTGCAAAAAGAGTTCATCAATAGAAACCTGGTGGAT
ACCCGGTACGCCACAgccGAGCTGACAAACTACCTGAAGGCCTACTTCAG
CGCCAACAATATGAACGTGAAGGTGAAAACGATCAACGGCAGCTTCACCG
ATTACCTGCGGAAAGTGTGGAAGTTTAAGAAGGAACGGAACCACGGCTAC
AAGCACCACGCCGAGGACGCCCTGATTATCGCTAATGCCGATTTCCTGTT
CAAAGAGAACAAGAAGCTGAAAGCCGTGAACTCTGTGCTGGAAAAACCTG
AGATCGAGAGCAAGCAGCTGGATATCCAGGTGGATAGCGAGGATAACTAC
AGCGAAATGTTCATCATCCCTAAGCAGGTCCAGGACATCAAGGACTTCAG
AAACTTCAAGTACAGCCACAGAGTGGACAAGAAGCCTAACAGACAGCTGA
TCAACGATACACTGTACAGCACCCGGAAGAAGGACAACTCCACCTACATC
GTGCAGACCATCAAAGATATCTATGCCAAAGATAATACCACCCTGAAGAA
GCAGTTTGACAAGTCACCCGAGAAGTTCCTCATGTACCAACACGATCCGC
GGACCTTCGAGAAGTTGGAAGTGATCATGAAGCAGTACGCTAATGAGAAG
AATCCTCTGGCCAAGTACCACGAGGAAACAGGCGAGTACCTGACCAAATA
CAGCAAAAAAAACAACGGCCCTATCGTGAAAAGCCTGAAGTACATTGGAA
ACAAGCTGGGCAGCCACCTAGATGTGACCCACCAGTTCAAGAGCAGCACC
AAGAAGITGGTGAAGCTGAGCATCAAGCCTTATAGATTCGACGTCTACCT
GACCGACAAGGGATATAAGTTCATCACCATCAGCTACCTGGACGTGCTGA
AGAAAGACAATTACTACTACATACCCGAACAGAAGTACGACAAGCTCAAA
CTGGGCAAGGCCATCGACAAAAACGCCAAGTTTATCGCTAGCTTCTACAA
GAATGATCTGATCAAGCTGGACGGCGAGATCTACAAGATCATCGGCGTGA
ATAGCGACACCAGAAACATGATCGAACTGGATCTGCCTGACATCAGATAC
AAAGAATACTGCGAGCTGAACAATATCAAGGGCGAACCTAGAATCAAAAA
GACCATCGGCAAAAAGGTGAATAGCATCGAAAAACTGACAACCGACGTGC
TGGGCAACGTGTTCACCAACACCCAGTACACAAAACCTCAGCTGCTGTTC
AAGCGAGGAAAT.

In one embodiment, the expression vector can be a plasmid vector, a retroviral vector, an adenovirus vector, an adeno-associated virus vector such as pAAV2_ITR, and the like. However, it can be understood that any other suitable expression vectors are also feasible.

In the present disclosure, any sgRNA for targeting can be designed for the DNA sequence to be edited according to actual needs, and the modification as known in the art can be made to the sgRNA to a certain extent. Therefore, in one embodiment, the modification to the sgRNA comprises, but is not limited to, phosphorylation, truncation, addition, sulfurization, methylation, and hydroxylation.

In the present disclosure, any mismatch sgRNA can be designed for the DNA sequence to be edited according to actual needs, and the modification as known in the art can be made to the sgRNA to a certain extent. The modification comprises, but is not limited to phosphorylation, truncation, addition, sulfurization, methylation, and hydroxylation.

In one embodiment, in step (3), the CRISPR/Cas9 system delivered to the cell containing the target site can comprises, but is not limited to, a plasmid, a retrovirus, an adenovirus, or an adeno-associated virus vector expressing the Cas9 protein and sgRNA of the present disclosure, or the sgRNA and the protein per se, depending on the actual needs.

In one further embodiment, in step (3), the delivery means comprise, but are not limited to, liposomes, cationic polymers, nanoparticles, multifunctional envelope-type nanoparticles, and viral vectors.

In one further embodiment, in step (3), the cell comprises, but is not limited to, eukaryotic cells and prokaryotic cells. The eukaryotic cells comprise, for example, mammalian cells and plant cells. The mammalian cells comprise, for example, animal cells, such as Chinese hamster ovary cells, baby hamster kidney cells, mouse Sertoli cells, mouse breast tumor cells, buffalo rat hepatic cells, rat hepatoma cells, SV40-transformed monkey kidney CVI lines, monkey kidney cells, canine kidney cells; and human cells, such as human cervical cancer cells, human lung cells, human liver cells, HIH/3T3 cells, human U2-OS osteosarcoma cells, human A549 cells, human K562 cells, human HEK293 cells, human HEK293T cells, human HCT116 cells, or human MCF-7 cells or TRI cells.

In one further embodiment, the modification in step (2) comprises, but is not limited to, phosphorylation, truncation, addition, sulfurization, or methylation.

In one particular embodiment, for other Cas9 genes than the SlugCas9-HF gene, the Oligo-F is SEQ ID NO: 16 and the Oligo-R is SEQ ID NO: 17; and for the SlugCas9-HF gene, the Oligo-F and the Oligo-R comprise a first oligo forward-strand sequence (Oligo-F1) and a first oligo reverse-strand sequence (Oligo-R1) represented by SEQ ID NO: 59 and SEQ ID NO: 60, respectively, and a second oligo forward-strand sequence (Oligo-F2) and a second oligo reverse-strand sequence (Oligo-R2) represented by SEQ ID NO: 61 and SEQ ID NO: 62, respectively.

As can be understood by those skilled in the art, the Oligo-F sequence and the Oligo-R sequence need to be annealed to become a double-stranded DNA. Therefore, in one embodiment, the annealing reaction comprises: 1 μL of 100 μM the oligo-F sequence, 1 μL of 100 μM the oligo-R sequence, and 28 μL of water. After shaking and mixing, the annealing reaction is placed in a PCR amplifier to run the annealing program as follows: 95ºC for 5 min, 85° ° C. for 1 min, 75° C. for 1 min, 65° C. for 1 min, 55° C. for 1 min, 45° ° C. for 1 min, 35° C. for 1 min, 25° C. for 1 min, and 4° C. for ever, with a cooling rate of 0.3° C./s.

In one embodiment, the expression vector cloned with Cas9, such as the plasmid pAAV2_Cas9_ITR, needs to be linearized with restriction endonuclease such as BsaI.

In one particular embodiment, the annealed product of the Oligo-F sequence and the Oligo-R sequence is ligated with the linearized expression vector cloned with Cas9, such as the pAAV2_Cas9_ITR backbone vector, via DNA ligase. In this way, an expression vector cloned with both Cas9 and sgRNA, such as pAAV2_Cas9-hU6-sgRNA, is obtained. In one more particular embodiment, the pAAV2_Cas9-hU6-sgRNA is an adeno-associated virus backbone plasmid, comprising AAV2 ITR, a CMV enhancer, a CMV promoter, SV40 NLS, Cas9, nucleoplasmin NLS, 3×HA, bGH poly(A), a human U6 promoter, a BsaI endonuclease site, and a sgRNA scaffold sequence.

In one particular embodiment, the ligation product is transformed into competent cells, and then subjected to Sanger sequencing for correct clone verification. Then the plasmid is extracted for use.

In one particular embodiment, for other Cas9 genes than the SlugCas9-HF gene, the cells in step (3) are HEK293T cells which contain a target site having the nucleotide sequence represented by SEQ ID NO: 18; and for the SlugCas9-HF gene, the target sites in the cell in step (3) have nucleotide sequences represented by SEQ ID NO: 63 and SEQ ID NO: 64, respectively.

In one particular embodiment, the delivery means in step (3) is a liposome, comprising, for example, Lipofectamine® 2000 or PEI.

In one optional embodiment, the method further comprises step (4) of detecting the editing efficiency for the edited target site, for example, by PCR amplification of the edited target site, followed by T7EI digestion or next-generation sequencing.

In one more particular embodiment, the template for PCR amplification in step (4) is edited genome DNA in HEK293T cells.

In one particular embodiment, in step (4), for other Cas9 genes than the SlugCas9-HF gene, the primer sequences for PCR amplification are represented by SEQ ID NO: 19, SEQ ID NO: 20, SEQ ID NO: 21, and SEQ ID NO: 22; and for the SlugCas9-HF gene, the primer sequences for PCR amplification are represented by SEQ ID NO: 65, SEQ ID NO: 66, SEQ ID NO: 21, SEQ ID NO: 22, and SEQ ID NO: 67.

In the third aspect, the present disclosure further provides a kit of a CRISPR/Cas9 gene editing system for gene editing, the kit comprises:
(1) a Cas9 protein and a sgRNA, wherein the Cas9 protein is:
a SauriCas9 protein having an amino acid sequence represented by SEQ ID NO: 1,
a ShaCas9 protein having an amino acid sequence represented by SEQ ID NO: 2,
a SlugCas9 protein having an amino acid sequence represented by SEQ ID NO: 3,
a SlutCas9 protein having an amino acid sequence represented by SEQ ID NO: 4,
a Sa-SauriCas9 protein having an amino acid sequence represented by SEQ ID NO: 5,
a Sa-SepCas9 protein having an amino acid sequence represented by SEQ ID NO: 6,
a Sa-SeqCas9 protein having an amino acid sequence represented by SEQ ID NO: 7,
a Sa-ShaCas9 protein having an amino acid sequence represented by SEQ ID NO: 8,
a Sa-SlugCas9 protein having an amino acid sequence represented by SEQ ID NO: 9,
a Sa-SlutCas9 protein having an amino acid sequence represented by SEQ ID NO: 10,
a SlugCas9-HF protein having an amino acid sequence represented by SEQ ID NO: 58, or
the Cas9 protein having an amino acid sequence that is at least 80% identical to the amino acid sequence represented by any one of SEQ ID NOs: 1-10 and SEQ ID NO: 58; and
   the sgRNA has a nucleotide sequence represented by SEQ ID NO: 11, or is a modified sgRNA sequence based on SEQ ID NO: 11; or
(2) an expression vector cloned with a Cas9 gene sequence and an oligo single-stranded DNA corresponding to the sgRNA, wherein
the Cas9 gene sequence:
   (a) has a nucleotide sequence encoding an amino acid sequence represented by any one of SEQ ID NO: 1-10 and SEQ ID NO: 58;
   (b) has a nucleotide sequence encoding an amino acid sequence that is at least 80% identical to the amino acid sequence represented by any one of SEQ ID NO: 1-10 and SEQ ID NO: 58; or
   (c) is a humanized Cas9 gene sequence, for example, a nucleotide sequence represented by any one of SEQ ID NOs: 23-32 and SEQ ID NO: 112; and
the sgRNA has a nucleotide sequence represented by SEQ ID NO: 11, or a modified sgRNA sequence based on SEQ ID NO: 11.

In the fourth aspect, the present disclosure provides use of the CRISPR/Cas9 system for gene editing according to the first aspect of the present disclosure in gene knockout, site-directed base change, site-directed insertion, regulation of gene transcription levels, regulation of DNA methylation, modification of DNA acetylation, modification of histone acetylation, single-base edition, or chromatin imaging tracking.

Compared with the existing CRISPR/Cas9 gene editing system in the prior art, the CRISPR/Cas9 gene editing system of the present disclosure comprises a smaller Cas9 protein with less amino acids than the prior art, and thus can be effectively packaged. Furthermore, the CRISPR/Cas9 gene editing system of the present disclosure can recognize a relatively simple PAM sequence, and thus can target more DNA sequences in the genome and has higher editing efficiency.

Hereinafter, the present disclosure will be described in more detail through the following examples with reference to the accompanying drawings. It should be understood that, unless otherwise specified, the reagents, methods, and devices used in the present disclosure are all conventional reagents, methods, and devices in the technical field. Unless otherwise specified, the reagents and materials used in the following examples are all commercially available. Experimental methods for which specific conditions are not indicated herein are usually carried out under the conditions conventional or recommended by the manufacturer(s).

### Example 1. Construction of Plasmid pAAV2_Cas9-ITR

Step (1): The amino acid sequences were downloaded according to the accession numbers of the Cas9 genes on UniProt.

In the present disclosure, the amino acid sequences for SauriCas9 gene, ShaCas9 gene, SlugCas9 gene, SlutCas9 gene, Sa-SauriCas9 gene, Sa-SepCas9 gene, Sa-SeqCas9 gene, Sa-ShaCas9 gene, Sa-SlugCas9 gene and Sa-SlutCas9 gene were downloaded according to the accession numbers of these genes on UniProt. The accession numbers of the Cas9 genes on UniProt and the amino acid sequences thereof are as follows.

| Cas9 gene | accession number | amino acid sequence number |
|---|---|---|
| SauriCas9 | A0A2T4M4R5 | SEQ ID NO: 1 |
| ShaCas9 | A0A2T4SLN6 | SEQ ID NO: 2 |
| SlugCas9 | A0A133QCR3 | SEQ ID NO: 3 |
| SlutCas9 | A0A1W6BMI2 | SEQ ID NO: 4 |
| Sa-SauriCas9 | A0A2T4M4R5 | SEQ ID NO: 5 |
| Sa-SepCas9 | A0A1Q9MLU4 | SEQ ID NO: 6 |
| Sa-SeqCas9 | A0A1E5TL62 | SEQ ID NO: 7 |
| Sa-ShaCas9 | A0A2T4SLN6 | SEQ ID NO: 8 |
| Sa-SlugCas9 | A0A133QCR3 | SEQ ID NO: 9 |
| Sa-SlutCas9 | A0A1W6BMI2 | SEQ ID NO: 10 |
| SlugCas9-HF | A0A133QCR3 | SEQ ID NO: 58* |

*Compared with SEQ ID NO: 3, R247A, N415A, T421A, and R656A mutations were introduced in SEQ ID NO: 58.

Step (2): The nucleotide sequences encoding the Cas9 proteins as specified above were subjected to codon optimization to obtain the coding sequences that highly express the Cas9 proteins in human cells. The coding sequences that highly express SauriCas9 protein, ShaCas9 protein, SlugCas9 protein, SlutCas9 protein, Sa-SauriCas9 protein Sa-SepCas9 protein, Sa-SeqCas9 protein, Sa-ShaCas9 protein, Sa-SlugCas9 protein and Sa-SlutCas9 protein in human cells are represented by SEQ ID NOs: 23-32 and SEQ ID NO: 112, respectively.

Step (3): The Cas9-coding sequences obtained in step (2) were subjected to gene synthesis and constructed into the pAAV2_ITR backbone plasmid to obtain the plasmids pAAV2_Cas9_ITR, as shown in FIG. 2.

### Example 2. Preparation of Linearized Plasmids pAAV2_Cas9-ITR

Step (1): The plasmids pAAV2_Cas9_ITR obtained in Example 1 were linearized by digestion with BsaI restriction endonuclease. The digestion mixtures each comprised 1 μg a plasmid pAAV2_Cas9_ITR, 5 μL 10× CutSmart buffer, 1 μL BsaI endonuclease, ddH₂O to 50 HL. The digestion mixtures were allowed to react at 37ºC for 1 hour.

Step (2): The digested products were subjected to electrophoresis on 1% agarose gel at 120 V for 30 minutes.

Step (3): The DNA fragments were cut, recovered by using a kit for gel recovery according to the instructions provided by the manufacturer, and finally eluted with ddH₂O. The DNA fragments as recovered were exactly the linearized plasmids pAAV2_Cas9_ITR comprising SauriCas9, ShaCas9, SlugCas9, SlutCas9, Sa-Sauri, Sa-SepCas9. Sa-SeqCas9, Sa-ShaCas9, Sa-SlugCas9, Sa-SlutCas9 and SlugCas9-HF with a size of 7447 bp, 7430 bp, 7427 bp, 7437 bp, 7433 bp, 7430 bp, 7423 bp, 7430 bp, 7430 bp, 7433 bp and 7427 bp, respectively.

Step (4): The recovered linearized plasmids pAAV2_Cas9_ITR were measured for the DNA concentration by using NanoDrop, and were stored for further use or were placed at −20° ° C. for long-term storage.

### Example 3. Construction of Plasmids pAAV2_Cas9_hU6_sgRNA

Step (1): The sgRNA sequence was designed.

Step (2): The sticky end sequences corresponding to both ends of the linearized plasmids pAAV2_Cas9_ITR were added to the sense strand and antisense strand corresponding to the designed sgRNA sequence, and oligo single-stranded DNAs were synthesized.

For genes other than SlugCas9-HF, the particular sequences of the oligo single-stranded DNAs were as follows:

| | Oligo-F: |
|---|---|
| (SEQ ID NO: 16) | |
| | CACCGCTCGGAGATCATCATTGCG; |
| | and |
| | Oligo-R: |
| (SEQ ID NO: 17) | |
| | AAACCGCAATGATGATCTCCGAGC. |

Additionally, for SlugCas9-HF, the particular sequences of the oligo single-stranded DNAs were as follows:

| | Oligo-F1: |
|---|---|
| (SEQ ID NO: 59) | |
| | CACCAGAGTAGGCTGGTAGATGGAG; |
| | Oligo-R1: |
| (SEQ ID NO: 60) | |
| | AAACCTCCATCTACCAGCCTACTCT; |
| | Oligo-F2: |
| (SEQ ID NO: 61) | |
| | CACCGTCAGACATGAGATCACAGAT; |
| | Oligo-R2: |
| (SEQ ID NO: 62) | |
| | AAACATCTGTGATCTCATGTCTGAC. |

Step (3): The oligo single-stranded DNAs were annealed to become double-stranded DNAs. The annealing reactions each comprised: 1 μL of 100 μM oligo-F, 1 μL of 100 μM oligo-R, and 28 μL ddH₂O. After being mixed by shaking, the annealing reactions were placed in a PCR amplifier to run the annealing program as follows: 95ºC for 5 min, 85ºC for 1 min, 75° ° C. for 1 min, 65° ° C. for 1 min, 55° C. for 1 min, 45° C. for 1 min, 35° C. for 1 min, 25° C. for 1 min, and at 4ºC for ever, with a cooling rate of 0.3ºC/s.

Step (4): The annealed products were ligated with the linearized plasmids pAAV2_Cas9_ITR obtained in Example 2 by using DNA ligase according to the instructions provided by the manufacturer.

Step (5): The ligated products 1 μL were taken for chemically competent transformation, and the grown bacterial clones were verified by Sanger sequencing.

Step (6): The correctly ligated clones verified by sequencing were cultured under shaking, and then used to extract the plasmids pAAV2 Cas9-hU6-sgRNA for further use.

### Example 4. Transfection of HEK293T Cell Line with Plasmids pAAV2_Cas9-hU6-sgRNA Expressing Cas9 Protein and sgRNA

1. Transfection of the GFP-reporter HEK293T cell line with the plasmids pAAV2_Cas9-hU6-sgRNA

Step (1): On day 0, according to the requirements of transfection, the GFP-reporter HEK293T cell line was plated on a 6-well plate at a cell density of about 30%. The sequence of the target site is represented by SEQ ID NO: 18 (GCTCGGAGATCATCATTGCGNNNNN).

Step (2): On day 1, transfection was performed by the following steps:
i. Plasmids to be transfected, pAAV2_Cas9-hU6-sgRNA, 2 μg, were taken and added into 100 μL Opti-MEM medium, and mixed homogenously by gently pipetting;
ii. 5 μL of flick mixed Lipofectamine® 2000 was pipetted to 100 μL Opti-MEM medium, mixed gently, and left to stand at room temperature for 5 min; and
iii. the diluted Lipofectamine® 2000 and the diluted plasmid were mixed homogenously by gently pipetting, left to stand at room temperature for 20 minutes, and then added into the medium containing the cells to be transfected. The cells to be transfected contained the CMV-ATG-target site-NNNNNNN-GFP nucleotide sequence represented by SEQ ID NO: 113, which comprises the sequence represented by SEQ ID NO: 18. It should be noted that the nucleotide sequence has 7 random bases N as the PAM sequence (marked in bold and underlined) between the target site and the GFP sequence.

Step (3): The cells were continued to be cultured in a 37° ° C., 5% CO₂ incubator.

Step (4): After being edited for 3 days, GFP-positive cells were sorted out by flow sorting, and continued to be cultured in a 37° C., 5% CO₂ incubator.

The sequence represented by SEQ ID NO: 113 is as follows:

GACATTGATTATTGACTAGTTATTAATAGTAATCAATTACGGGGTCATTA
GTTCATAGCCCATATATGGAGTTCCGCGTTACATAACTTACGGTAAATGG
CCCGCCTGGCTGACCGCCCAACGACCCCCGCCCATTGACGTCAATAATGA
CGTATGTTCCCATAGTAACGCCAATAGGGACTTTCCATTGACGTCAATGG
GTGGAGTATTTACGGTAAACTGCCCACTTGGCAGTACATCAAGTGTATCA
TATGCCAAGTACGCCCCCTATTGACGTCAATGACGGTAAATGGCCCGCCT
GGCATTATGCCCAGTACATGACCTTATGGGACTTTCCTACTTGGCAGTAC
ATCTACGTATTAGTCATCGCTATTACCATGGTGATGCGGTTTTGGCAGTA
CATCAATGGGCGTGGATAGCGGTTTGACTCACGGGGATTTCCAAGTCTCC
ACCCCATTGACGTCAATGGGAGTTTGTTTTGGCACCAAAATCAACGGGAC
TTTCCAAAATGTCGTAACAACTCCGCCCCATTGACGCAAATGGGCGGTAG
GCGTGTACGGTGGGAGGTCTATATAAGCAGTCTAGAGATCCGACGCCGCC
ATCTCTAGGCCCGCGCCGGCCCCCTCGCACAGACTTGTGGGAGAAGCTCG
GCTACTCCCCTGCCCCGGTTAATTTGCATATAATATTTCCTAGTAACTAT
AGAGGCTTAATGTGCGATAAAAGACAGATAATCTGTTCTTTTTAATACTA
GCTACATTTTACATGATAGGCTTGGATTTCTATAAGAGATACAAATACTA
AATTATTATTTTAAAAAACAGCACAAAAGGAAACTCACCCTAACTGTAAA
GTAATTGTGTGTTTTGAGACTATAAATATGCATGCGAGAAAAGCCTTGTT
TGCCACCATGGAACGGCTCGGAGATCATCATTGCGNNNNNNNGTGAGCAA
GGGCGAGGAGCTGTTCACCGGGGTGGTGCCCATCCTGGTCGAGCTGGACG
GCGACGTAAACGGCCACAAGTTCAGCGTGTCCGGCGAGGGCGAGGGCGAT
GCCACCTACGGCAAGCTGACCCTGAAGTTCATCTGCACCACCGGCAAGCT
GCCCGTGCCCTGGCCCACCCTCGTGACCACCCTGACCTACGGCGTGCAGT
GCTTCAGCCGCTACCCCGACCACATGAAGCAGCACGACTTCTTCAAGTCC
GCCATGCCCGAAGGCTACGTCCAGGAGCGCACCATCTTCTTCAAGGACGA
CGGCAACTACAAGACCCGCGCCGAGGTGAAGTTCGAGGGCGACACCCTGG
TGAACCGCATCGAGCTGAAGGGCATCGACTTCAAGGAGGACGGCAACATC
CTGGGGCACAAGCTGGAGTACAACTACAACAGCCACAACGTCTATATCAT
GGCCGACAAGCAGAAGAACGGCATCAAGGTGAACTTCAAGATCCGCCACA
ACATCGAGGACGGCAGCGTGCAGCTCGCCGACCACTACCAGCAGAACACC
CCCATCGGCGACGGCCCCGTGCTGCTGCCCGACAACCACTACCTGAGCAC
CCAGTCCAAGCTGAGCAAAGACCCCAACGAGAAGCGCGATCACATGGTCC
TGCTGGAGTTCGTGACCGCCGCCGGGATCACTCTCGGCATGGACGAGCTG
TACAAG

2. Transfection of HEK293T cell line with pAAV2_SlugCas9-HF-hU6-sgRNA

Step (1): On day 0, according to the requirements of transfection, the HEK293T cell line containing the sgRNA target site was plated on a 6-well plate at a cell density of about 30%. The sequences of the G4 and G7 target sites for SlugCas9-HF are represented by SEQ ID NO: 63 (AGAGTAGGCTGGTAGATGGAGNNNN) and SEQ ID NO: 64 (ATCTGTGATCTCATGTCTGACNNNN), respectively.

Step (2): On day 1, transfection was performed via the following steps:
i. 2 μg of the plasmid to be transfected, pAAV2_SlugCas9-HF-hU6-sgRNA, was taken and added into 100 μL Opti-MEM medium, and mixed homogenously by gently pipetting;
ii. 5 μL of flick mixed Lipofectamine® 2000 was pipetted to 100 μL Opti-MEM medium, mixed gently, and left to stand at room temperature for 5 min; and
iii. The diluted Lipofectamine® 2000 and the diluted plasmid were mixed homogenously by gently pipetting, left to stand at room temperature for 20 minutes, and then added into the medium containing the cells to be transfected.

Step (3): The cells were continued to be cultured in a 37° C., 5% CO₂ incubator.

### Example 5. Preparation of Next-Generation Sequencing Library

Step (1): HEK293T cells after being edited for 3 days or the GFP-reporter HEK293T cell line after flow sorting were collected and used to extract the genome DNA by using a DNA kit according to the instructions provided by the manufacturer.

Step (2): The first round of PCR for PCR library construction was performed with 2×Q5 Mastermix. For genes other than SlugCas9-HF, the PCR primers are represented by SEQ ID NO: 19 and SEQ ID NO: 20; and for SlugCas9-HF, the PCR primers are represented by SEQ ID NO: 65 and SEQ ID NO: 66, as follows:

F1-
(SEQ ID NO: 19)
ACACTCTTTCCCTACACGACGCTCTTCCGATCTNNNNGCGAGAAAAGCCT
TGTTT;
R1-
(SEQ ID NO: 20)
ACTGGAGTTCAGACGTGTGCTCTTCCGATCTCTGAACTTGTGGCCGTTTA
C;
F1-
(SEQ ID NO: 65)
ACACTCTTTCCCTACACGACGCTCTTCCGATCTNNNNTGTCAGGCAGCAG
AGCTC;
R1-
(SEQ ID NO: 66)
ACTGGAGTTCAGACGTGTGCTCTTCCGATCTNNNNGGCGATGGCTTCCTG
GTC.

The reaction was as follows:

| Reagents | 25 μL reaction | |
|---|---|---|
| 10 μM F1 | 1.25 | μL |
| 10 μM R1 | 1.25 | μL |
| 2X Q5 master mix | 12.5 | μL |
| genomic DNA | 5 | μg |
| ddH₂O | to 25 | μL |

The PCR procedure was as follows:

| | | | | |
|---|---|---|---|---|
| Initial denaturation | 98° | C. | 2 | min |
| 35 cycles | 95° | C. | 7 | s |
| | 65° | C. | 20 | s |
| | 72° | C. | 10 | s |
| Final extension | 72° | C. | 2 | min |

| | | | |
|---|---|---|---|
| Hold | 4° | C. | ∞ |

Step (3): The second round of PCR for PCR library construction was performed with 2×Q5 Mastermix. For genes other than SlugCas9-HF, the PCR primers are represented by SEQ ID NO: 21 and SEQ ID NO: 22; and for SlugCas9-HF, the products from the first round of PCR for the G4 site were amplified with the primers represented by SEQ ID NO: 21 and SEQ ID NO: 22; and the products from the first round of PCR for the G7 site were amplified with the primers represented by SEQ ID NO: 21 and SEQ ID NO: 67.

F2-
(SEQ ID NO: 21)
AATGATACGGCGACCACCGAGATCTACACTCTTTCCCTACACGAC
R2-
(SEQ ID NO: 22)
CAAGCAGAAGACGGCATACGAGATCACTGTGTGACTGGAGTTCAGACGTG
TG
F2-
(SEQ ID NO: 21)
AATGATACGGCGACCACCGAGATCTACACTCTTTCCCTACACGAC
R2-1-
(SEQ ID NO: 22)
CAAGCAGAAGACGGCATACGAGATCACTGTGTGACTGGAGTTCAGACGTG
TG
F2-
(SEQ ID NO: 21)
AATGATACGGCGACCACCGAGATCTACACTCTTTCCCTACACGAC
R2-2-
(SEQ ID NO: 67)
CAAGCAGAAGACGGCATACGAGATATTGGCGTGACTGGAGTTCAGACGTG
TG

The reaction was as follows:

| Reagents: | 25 μL reaction | |
|---|---|---|
| 10 μM F2 | 1.25 | μL |
| 10 μM R2 | 1.25 | μL |
| 2X Q5 master mix | 12.5 | μL |
| The products from the first round | 3 | μL |
| ddH₂O | 7 | μL |

The PCR procedure was as follows:

| | | | | |
|---|---|---|---|---|
| Initial denaturation | 98° | C. | 30 | s |
| 15 cycles | 95° | C. | 7 | s |
| | 65° | C. | 20 | s |
| | 72° | C. | 10 | s |
| Final extension | 72° | C. | 2 | min |

| | | | |
|---|---|---|---|
| Hold | 4° | C. | ∞ |

Step (4): The products from the second round of PCR were purified with a kit for gel recovery according to the instructions provided by the manufacturer to obtain the DNA fragments with a size of 366 bp or 406 bp (the latter is only for SlugCas9-HF), thereby completing the preparation of the library for next-generation sequencing.

### Example 6. Analysis of Next-Generation Sequencing Results

Step (1): The prepared library for next-generation sequencing was subjected to paired-end sequencing on HiseqXTen.

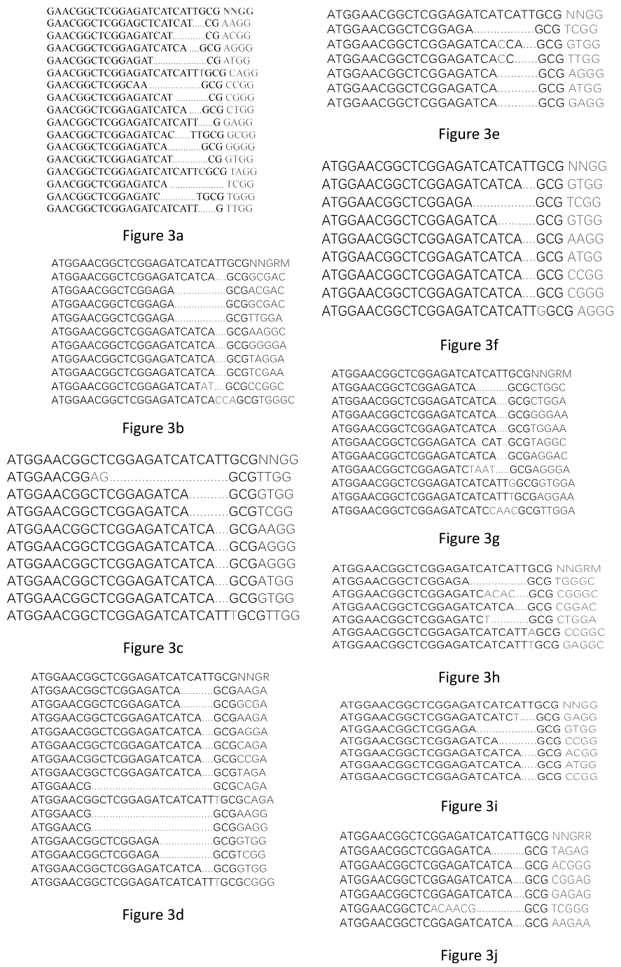

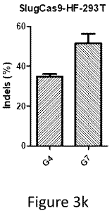

Step (2): The next-generation sequencing results were analyzed via Bioinformatics. Some of the editing results are shown in FIG. 3a to FIG. 3j. It can be seen from the figures that the editing results comprise deletions, insertions or mismatches, and the last 4 bp or 5 bp represents the PAM sequence, which is NNGG, NNGRM, NNGG, NNGR, NNGG, NNGG, NNGRM, NNGRM, NNGG and NNGRR, respectively, from FIGS. 3a to 3j. FIG. 3k shows the editing of the SlugCas9-HF gene editing system at two target sites, where the X axis represents the G4 and G7 target sites, and the Y axis represents the indel efficiency.

### Example 7. Verification of Endogenous Sites

Step (1): The plasmids pAAV2_Cas9-hU6-sgRNA expressing Cas9 and sgRNA were transfected into HEK293T cells by Lipofectamine® 2000 according to the instructions provided by the manufacturer. The particular sequences for different Cas9s, crRNAs and target sites are given in Table 1.

Step (2): The genomic DNA in cells which had been edited for 5 days was extracted, and the target DNA sequence was amplified with 2×Q5 Master mix and Test-F and Test-R primers. The detailed sequences of the Test-F and Test-R primers are given in Table 1 below.

Step (3): The PCR products were recovered and purified by agarose gel electrophoresis, to obtain the DNA fragments of different sizes. The sizes of the DNA fragments are shown in Table 1.

Step (4): The purified DNA fragments were digested according to the instructions for T7 Endonuclease I, and then detected by gel electrophoresis.

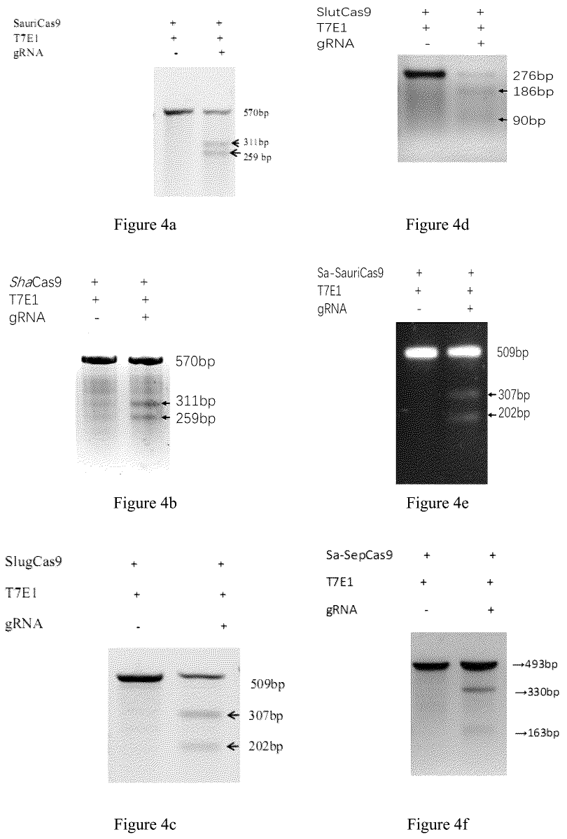

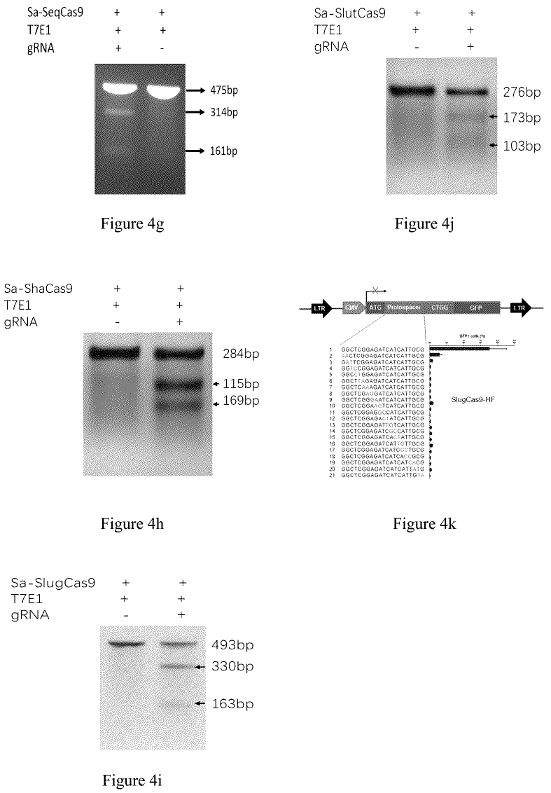

The results are shown in FIGS. 4a-4j. In each figure, the left lane corresponds to the negative control group which did not involve sgRNA during transfection, and there was no cleaved fragment after the target sequence was cleaved by T7 Endonuclease I, indicating that no editing has occurred; and the right lane corresponds to the treatment group, which involved sgRNA during transfection, and the cleaved fragments appeared after the target sequence was cleaved by T7 Endonuclease I, indicating that editing has occurred.

TABLE 1

| Serial | | | | | DNA |
|---|---|---|---|---|---|
| number | Cas9 genes | CrRNAs | target sites | Test-F and Test-R | fragments |
| 1 | SauriCas9 | AGATGCGGGTGATGATG | AGATGCGGGTGATGATG | CAGGGAGTCGACGAGTTGAA | 570 bp |
| | | CTCT | CTCTTTGG | (SEQ ID NO: 47); | |
| | | (SEQ ID NO: 33) | (SEQ ID NO: 40) | TAATTGCTGGCCTATCCACG | |
| | | | | C (SEQ ID NO: 53) | |
| 2 | ShaCas9 | AGATGCGGGTGATGATG | AGATGCGGGTGATGATG | CAGGGAGTCGACGAGTTGAA | 570 bp |
| | | CTCT | CTCTTTGG | (SEQ ID NO: 47); | |
| | | (SEQ ID NO: 33) | (SEQ ID NO: 40) | TAATTGCTGGCCTATCCACG | |
| | | | | C (SEQ ID NO: 53); | |
| 3 | SlugCas9 | ATAGGGTTAGGGGCCCC | ATAGGGTTAGGGGCCCC | ACGCAGTGGGTCATAGGCTC | 509 bp |
| | | AGGC | AGGCCGGG | (SEQ ID NO: 48); | |
| | | (SEQ ID NO: 34) | (SEQ ID NO: 41) | GGACTCAGGCCCTTCCTCCT | |
| | | | | (SEQ ID NO: 54) | |
| 4 | SlutCas9 | GGCGCAGTTTACTGCAC | GGCGCAGTTTACTGCAC | AGGGAAGAGGAAATGCTGGG | 276 bp |
| | | AGGT | AGGTGCGG | (SEQ ID NO: 49); | |
| | | (SEQ ID NO: 35) | (SEQ ID NO: 42) | TGAGCCGCCAGTGTACAGA | |
| | | | | (SEQ ID NO: 55) | |
| 5 | Sa-SauriCas9 | ATAGGGTTAGGGGCCCC | ATAGGGTTAGGGGCCCC | ACGCAGTGGGTCATAGGCTC | 509 bp |
| | | AGGC | AGGCCGGG | (SEQ ID NO: 48); | |
| | | (SEQ ID NO: 34) | (SEQ ID NO: 41) | GGACTCAGGCCCTTCCTCCT | |
| | | | | (SEQ ID NO: 54) | |
| 6 | Sa-SepCas9 | GGGAAGAGTGAGGGGAA | GGGAAGAGTGAGGGGAA | TCCTTGAACAGCCTGCAAAC | 493 bp |
| | | CAAA | CAAAGTGG | (SEQ ID NO: 50); | |
| | | (SEQ ID NO: 36) | (SEQ ID NO: 43) | AAGGAGGTCTCTGTCTGTGC | |
| | | | | (SEQ ID NO: 56) | |
| 7 | Sa-SeqCas9 | GAGCTGGTGGACCTAGT | GAGCTGGTGGACCTAGT | ATCAACCCGGAGCAGATTC | 475 bp |
| | | ACA | ACAATGGA | (SEQ ID NO: 51); | |
| | | (SEQ ID NO: 37) | (SEQ ID NO: 44) | CCTCATTGTCCAGAAAGACC | |
| | | | | A (SEQ ID NO: 57) | |
| 8 | Sa-ShaCas9 | AGTGAGGGGAACAAAGT | AGTGAGGGGAACAAAGT | GCTGCTTTCCTGCTGTCTTC | 284 bp |
| | | GGAC | GGACATGGC | (SEQ ID NO: 52); | |
| | | (SEQ ID NO: 38) | (SEQ ID NO: 45) | AAGGAGGTCTCTGTCTGTGC | |
| | | | | (SEQ ID NO: 56) | |
| 9 | Sa-SlugCas9 | GGGAAGAGTGAGGGGAA | GGGAAGAGTGAGGGGAA | TCCTTGAACAGCCTGCAAAC | 493 bp |
| | | CAAA | CAAAGTGG | (SEQ ID NO: 50); | |
| | | (SEQ ID NO: 36) | (SEQ ID NO: 43) | AAGGAGGTCTCTGTCTGTGC | |
| | | | | (SEQ ID NO: 56) | |
| 10 | Sa-SlutCas9 | GCGAGCCTGAGGCGAAC | GCGAGCCTGAGGCGAAC | AGGGAAGAGGAAATGCTGGG | 276 bp |
| | | AATG | AATGGCGGA | (SEQ ID NO: 49); | |
| | | (SEQ ID NO: 39) | (SEQ ID NO: 46) | TGAGCCGCCAGTGTACAGA | |
| | | | | (SEQ ID NO: 55) | |

### Example 8. Detection of CRISPR/Cas9 System Specificity

In this example, SlugCas9-HF was taken as an example to verify the specificity of the CRISPR/Cas9 system. The particular protocols were as follows:
1. Plasmid pAAV2_SlugCas9-HF_ITR was constructed by following the protocols in Example 1.
2. Linearized plasmid pAAV2_SlugCas9-HF_ITR was prepared by following the protocols in Example 2.
3. Plasmids pAAV2_SlugCas9-HF-hU6-on target sgRNA and pAAV2_SlugCas9-HF-hU6-mismatch sgRNA were constructed.

Step (1): The on-target sgRNA sequence and the mismatch sgRNA sequences were designed.

Step (2): The sticky end sequences corresponding to both ends of the linearized plasmid pAAV2_SlugCas9-HF_ITR were added to the sense strand and antisense strand corresponding to the designed on-target sgRNA sequence and the mismatch sgRNA sequence, and oligo single-stranded DNAs were synthesized. The particular sequences are as follows (wherein the bases underlined and in bold are mismatch bases):

| | Oligo-F3: |
|---|---|
| (SEQ ID NO: 68) | |
| | CACCGGCTCGGAGATCATCATTGCG |
| | (on-target) |
| | Oligo-R3: |
| (SEQ ID NO: 89) | |
| | AAACCGCAATGATGATCTCCGAGCC |
| | (on-target) |
| | Oligo-F4: |
| (SEQ ID NO: 69) | |
| | CACCAACTCGGAGATCATCATTGCG |
| | (mismatch) |
| | Oligo-F5: |
| (SEQ ID NO: 70) | |
| | CACCGATTCGGAGATCATCATTGCG |
| | (mismatch) |
| | Oligo-F6: |
| (SEQ ID NO: 71) | |
| | CACCGGTCCGGAGATCATCATTGCG |
| | (mismatch) |
| | Oligo-F7: |
| (SEQ ID NO: 72) | |
| | CACCGGCCTGGAGATCATCATTGCG |
| | (mismatch) |
| | Oligo-F8: |
| (SEQ ID NO: 73) | |
| | CACCGGCTTAGAGATCATCATTGCG |
| | (mismatch) |
| | Oligo-F9: |
| (SEQ ID NO: 74) | |
| | CACCGGCTCAAAGATCATCATTGCG |
| | (mismatch) |
| | Oligo-F10: |
| (SEQ ID NO: 75) | |
| | CACCGGCTCGAGGATCATCATTGCG |
| | (mismatch) |
| | Oligo-F11: |
| (SEQ ID NO: 76) | |
| | CACCGGCTCGGGAATCATCATTGCG |
| | (mismatch) |
| | Oligo-F12: |
| (SEQ ID NO: 77) | |
| | CACCGGCTCGGAAGTCATCATTGCG |
| | (mismatch) |
| | Oligo-F13: |
| (SEQ ID NO: 78) | |
| | CACCGGCTCGGAGGCCATCATTGCG |
| | (mismatch) |
| | Oligo-F14: |
| (SEQ ID NO: 79) | |
| | CACCGGCTCGGAGACTATCATTGCG |
| | (mismatch) |
| | Oligo-F15: |
| (SEQ ID NO: 80) | |
| | CACCGGCTCGGAGATTGTCATTGCG |
| | (mismatch) |
| | Oligo-F16: |
| (SEQ ID NO: 81) | |
| | CACCGGCTCGGAGATCGCCATTGCG |
| | (mismatch) |
| | Oligo-F17: |
| (SEQ ID NO: 82) | |
| | CACCGGCTCGGAGATCACTATTGCG |
| | (mismatch) |
| | Oligo-F18: |
| (SEQ ID NO: 83) | |
| | CACCGGCTCGGAGATCATTGTTGCG |
| | (mismatch) |
| | Oligo-F19: |
| (SEQ ID NO: 84) | |
| | CACCGGCTCGGAGATCATCGCTGCG |
| | (mismatch) |
| | Oligo-F20: |
| (SEQ ID NO: 85) | |
| | CACCGGCTCGGAGATCATCACCGCG |
| | (mismatch) |
| | Oligo-F21: |
| (SEQ ID NO: 86) | |
| | CACCGGCTCGGAGATCATCATCACG |
| | (mismatch) |
| | Oligo-F22: |
| (SEQ ID NO: 87) | |
| | CACCGGCTCGGAGATCATCATTATG |
| | (mismatch) |
| | Oligo-F23: |
| (SEQ ID NO: 88) | |
| | CACCGGCTCGGAGATCATCATTGTA |
| | (mismatch) |
| | Oligo-R4: |
| (SEQ ID NO: 90) | |
| | AAACCGCAATGATGATCTCCGAGTT |
| | (mismatch) |
| | Oligo-R5: |
| (SEQ ID NO: 91) | |
| | AAACCGCAATGATGATCTCCGAATC |
| | (mismatch) |
| | Oligo-R6: |
| (SEQ ID NO: 92) | |
| | AAACCGCAATGATGATCTCCGGACC |
| | (mismatch) |
| | Oligo-R7: |
| (SEQ ID NO: 93) | |
| | AAACCGCAATGATGATCTCCAGGCC |
| | (mismatch) |
| | Oligo-R8: |
| (SEQ ID NO: 94) | |
| | AAACCGCAATGATGATCTCTAAGCC |
| | (mismatch) |
| | Oligo-R9: |
| (SEQ ID NO: 95) | |
| | AAACCGCAATGATGATCTTTGAGCC |
| | (mismatch) |
| | Oligo-R10: |
| (SEQ ID NO: 96) | |
| | AAACCGCAATGATGATCCTCGAGCC |
| | (mismatch) |
| | Oligo-R11: |
| (SEQ ID NO: 97) | |
| | AAACCGCAATGATGATTCCCGAGCC |
| | (mismatch) |
| | Oligo-R12: |
| (SEQ ID NO: 98) | |
| | AAACCGCAATGATGACTTCCGAGCC |
| | (mismatch) |
| | Oligo-R13: |
| (SEQ ID NO: 99) | |
| | AAACCGCAATGATGGCCTCCGAGCC |
| | (mismatch) |
| | Oligo-R14: |
| (SEQ ID NO: 100) | |
| | AAACCGCAATGATAGTCTCCGAGCC |
| | (mismatch) |
| | Oligo-R15: |
| (SEQ ID NO: 101) | |
| | AAACCGCAATGACAATCTCCGAGCC |
| | (mismatch) |
| | Oligo-R16: |
| (SEQ ID NO: 102) | |
| | AAACCGCAATGGCGATCTCCGAGCC |
| | (mismatch) |
| | Oligo-R17: |
| (SEQ ID NO: 103) | |
| | AAACCGCAATAGTGATCTCCGAGCC |
| | (mismatch) |
| | Oligo-R18: |
| (SEQ ID NO: 104) | |
| | AAACCGCAACAATGATCTCCGAGCC |
| | (mismatch) |
| | Oligo-R19: |
| (SEQ ID NO: 105) | |
| | AAACCGCAGCGATGATCTCCGAGCC |
| | (mismatch) |
| | Oligo-R20: |
| (SEQ ID NO: 106) | |
| | AAACCGCGGTGATGATCTCCGAGCC |
| | (mismatch) |
| | Oligo-R21: |
| (SEQ ID NO: 107) | |
| | AAACCGTGATGATGATCTCCGAGCC |
| | (mismatch) |
| | Oligo-R22: |
| (SEQ ID NO: 108) | |
| | AAACCATAATGATGATCTCCGAGCC |
| | (mismatch) |
| | Oligo-R23: |
| (SEQ ID NO: 109) | |
| | AAACTACAATGATGATCTCCGAGCC. |
| | (mismatch) |

Step (3): The oligo single-stranded DNAs were annealed to be double-stranded DNAs, and the annealing reactions each comprised: 1 μL of 100 μM oligo-F, 1 μL of 100 μM oligo-R, and 28 μL ddH₂O. After being mixed by shaking, the annealing reactions were placed in a PCR amplifier to run the annealing program as follows: 95° C. for 5 min, 85° C. for 1 min, 75ºC for 1 min, 65° C. for 1 min, 55° ° C. for 1 min, 45° ° C. for 1 min, 35° C. for 1 min, 25° ° C. for 1 min, and 4° C. for ever, with a cooling rate of 0.3ºC/s.

Step (4): The annealed products were ligated with the linearized plasmid pAAV2_SlugCas9-HF_ITR by using DNA ligase according to the instructions provided by the manufacturer.

Step (5): The ligated products (1 μL) were taken for chemically competent transformation, and the grown bacterial clones were verified by Sanger sequencing.

Step (6): The correctly ligated clones verified by sequencing were cultured under shaking and then used to extract the plasmids, pAAV2_SlugCas9-HF-hU6-on target sgRNA and pAAV2_SlugCas9-HF-hU6-mismatch sgRNAs for further use.

4. The GFP-reporter HEK293T cell line was transfected with pAAV2_SlugCas9-HF-hU6-on target sgRNA and pAAV2_SlugCas9-HF-hU6-mismatch sgRNAs, respectively. The particular steps are as follows.

Step (1): On day 0, according to the requirements of transfection, the GFP-reporter HEK293T cell line was plated on a 6-well plate at a cell density of about 30%. The sequence of the target site is represented by SEQ ID NO: 110 (GGCTCGGAGATCATCATTGCGNNNN).

Step (2): On day 1, transfection was performed by the following steps:
i. the plasmids to be transfected, pAAV2_SlugCas9-HF-hU6-on target sgRNA/mismatch sgRNA, 2 μg, were taken and added into 100 μL Opti-MEM medium, and mixed homogenously by gently pipetting;
ii. 5 μL of flick mixed Lipofectamine® 2000 was pipetted to 100 μL Opti-MEM medium, mixed gently, and left to stand at room temperature for 5 min; and
iii. the diluted Lipofectamine® 2000 and the diluted plasmid were mixed homogenously by gently pipetting, left to stand at room temperature for 20 minutes, and then added into the medium containing the cells to be transfected. The cells to be transfected comprised the CMV-ATG-target site-CTGG-GFP nucleotide sequence represented by SEQ ID NO: 111, which comprised the sequence represented by SEQ ID NO: 110.

Step (3): The cells were continued to be cultured in a 37° C., 5% CO₂ incubator.

5. Analysis of the editing efficiency and off-target rate of SlugCas9-HF by flow cytometry.

Step (1): The cells edited for 3 days were collected, and were subjected to the flow cytometry.

Step (2): The GFP positive ratio was analyzed by using FlowJo analysis software and plotted.

The sequence represented by SEQ ID NO: 111 is as follows:

GACATTGATTATTGACTAGTTATTAATAGTAATCAATTACGGGGTCATTA
GTTCATAGCCCATATATGGAGTTCCGCGTTACATAACTTACGGTAAATGG
CCCGCCTGGCTGACCGCCCAACGACCCCCGCCCATTGACGTCAATAATGA
CGTATGTTCCCATAGTAACGCCAATAGGGACTTTCCATTGACGTCAATGG
GTGGAGTATTTACGGTAAACTGCCCACTTGGCAGTACATCAAGTGTATCA
TATGCCAAGTACGCCCCCTATTGACGTCAATGACGGTAAATGGCCCGCCT
GGCATTATGCCCAGTACATGACCTTATGGGACTTTCCTACTTGGCAGTAC
ATCTACGTATTAGTCATCGCTATTACCATGGTGATGCGGTTTTGGCAGTA
CATCAATGGGCGTGGATAGCGGTTTGACTCACGGGGATTTCCAAGTCTCC
ACCCCATTGACGTCAATGGGAGTTTGTTTTGGCACCAAAATCAACGGGAC
TTTCCAAAATGTCGTAACAACTCCGCCCCATTGACGCAAATGGGCGGTAG
GCGTGTACGGTGGGAGGTCTATATAAGCAGTCTAGAGATCCGACGCCGCC
ATCTCTAGGCCCGCGCCGGCCCCCTCGCACAGACTTGTGGGAGAAGCTCG
GCTACTCCCCTGCCCCGGTTAATTTGCATATAATATTTCCTAGTAACTAT
AGAGGCTTAATGTGCGATAAAAGACAGATAATCTGTTCTTTTTAATACTA
GCTACATTTTACATGATAGGCTTGGATTTCTATAAGAGATACAAATACTA
AATTATTATTTTAAAAAACAGCACAAAAGGAAACTCACCCTAACTGTAAA
GTAATTGTGTGTTTTGAGACTATAAATATGCATGCGAGAAAAGCCTTGTT
TGCCACCATGGGCTCGGAGATCATCATTGCGCTGGGTGAGCAAGGGCGAG
GAGCTGTTCACCGGGGTGGTGCCCATCCTGGTCGAGCTGGACGGCGACGT
AAACGGCCACAAGTTCAGCGTGTCCGGCGAGGGCGAGGGCGATGCCACCT
ACGGCAAGCTGACCCTGAAGTTCATCTGCACCACCGGCAAGCTGCCCGTG
CCCTGGCCCACCCTCGTGACCACCCTGACCTACGGCGTGCAGTGCTTCAG
CCGCTACCCCGACCACATGAAGCAGCACGACTTCTTCAAGTCCGCCATGC
CCGAAGGCTACGTCCAGGAGCGCACCATCTTCTTCAAGGACGACGGCAAC
TACAAGACCCGCGCCGAGGTGAAGTTCGAGGGCGACACCCTGGTGAACCG
CATCGAGCTGAAGGGCATCGACTTCAAGGAGGACGGCAACATCCTGGGGC
ACAAGCTGGAGTACAACTACAACAGCCACAACGTCTATATCATGGCCGAC
AAGCAGAAGAACGGCATCAAGGTGAACTTCAAGATCCGCCACAACATCGA
GGACGGCAGCGTGCAGCTCGCCGACCACTACCAGCAGAACACCCCCATCG
GCGACGGCCCCGTGCTGCTGCCCGACAACCACTACCTGAGCACCCAGTCC
AAGCTGAGCAAAGACCCCAACGAGAAGCGCGATCACATGGTCCTGCTGGA
GTTCGTGACCGCCGCCGGGATCACTCTCGGCATGGACGAGCTGTACAAG

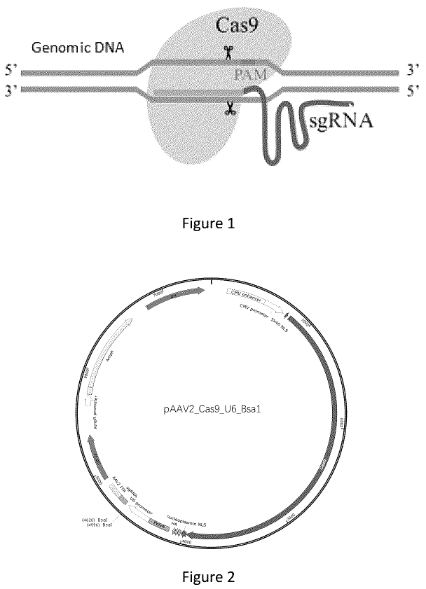

FIG. 4k shows the detection results of the specificity of the SlugCas9-HF gene editing system in the GFP-reporter HEK293T cell line. It can be seen from FIG. 4 that the complex of SlugCas9-HF and the sgRNA specifically cleaved the target sequence (the sequence shown in FIG. 1), but was essentially incapable of or was incapable of cleaving the non-target sequences (the sequences shown in FIG. 2-21). Accordingly, the system of the present disclosure shows high specificity and a low off-target rate.

1. A CRISPR/Cas9 system for gene editing in cells or in vitro, wherein the CRISPR/Cas9 system is a complex of a Cas9 protein and a sgRNA, which is capable of accurately locating and cleaving a target DNA sequence so as to cause double-strand break damage to the target DNA sequence wherein
the Cas9 protein is:
a SauriCas9 protein having an amino acid sequence represented by SEQ ID NO: 1,
a ShaCas9 protein having an amino acid sequence represented by SEQ ID NO: 2,
a SlugCas9 protein having an amino acid sequence represented by SEQ ID NO: 3,
a SlutCas9 protein having an amino acid sequence represented by SEQ ID NO: 4,
a Sa-SauriCas9 protein having an amino acid sequence represented by SEQ ID NO: 5,
a Sa-SepCas9 protein having an amino acid sequence represented by SEQ ID NO: 6,
a Sa-SeqCas9 protein having an amino acid sequence represented by SEQ ID NO: 7,
a Sa-ShaCas9 protein having an amino acid sequence represented by SEQ ID NO: 8,
a Sa-SlugCas9 protein having an amino acid sequence represented by SEQ ID NO: 9,
a Sa-SlutCas9 protein having an amino acid sequence represented by SEQ ID NO: 10,
a SlugCas9-HF protein having an amino acid sequence represented by SEQ ID NO: 58, or
a Cas9 protein having an amino acid sequence that is at least 80% identical to the amino acid sequence represented by any one of SEQ ID NOs: 1-10 and SEQ ID NO: 58; and
the sgRNA has a nucleotide sequence represented by SEQ ID NO: 11, or is a modified sgRNA sequence based on SEQ ID NO: 11.

2. The CRISPR/Cas9 system for gene editing according to claim 1, wherein the cell comprises eukaryotic cells and prokaryotic cells; the eukaryotic cells comprise mammalian cells and plant cells; the mammalian cells comprise Chinese hamster ovary cells, baby hamster kidney cells, mouse Sertoli cells, mouse breast tumor cells, buffalo rat hepatic cells, rat hepatoma cells, SV40-transformed monkey kidney CVI lines, monkey kidney cells, canine kidney cells, human cervical cancer cells, human lung cells, human liver cells, HIH/3T3 cells, human U2-OS osteosarcoma cells, human A549 cells, human K562 cells, human HEK293 cells, human HEK293T cells, human HCT116 cells, or human MCF-7 cells or TRI cells.

3. The CRISPR/Cas9 system for gene editing according to claim 1, wherein the Cas9 protein comprises a Cas9 protein that has no cleavage activity, or only has single-stranded cleavage activity, or has double-stranded cleavage activity.

4. The CRISPR/Cas9 system for gene editing according to claim 1, wherein accurately locating the target DNA sequence comprises recognizing, by the complex of the Cas9 protein and the sgRNA, a PAM sequence on the target DNA sequence, and forming a complementary base pairing structure from a 20 bp or 21 bp sequence at the 5′ end of the sgRNA and the target DNA sequence.

5. (canceled)

6. (canceled)

7. (canceled)

8. The CRISPR/Cas9 system for gene editing according to claim 4, wherein:
for the SauriCas9 protein, the PAM is NNGG, and the target DNA sequence is represented by SEQ ID NO: 12;
for the ShaCas9 protein, the PAM is NNGRM, and the target DNA sequence is represented by SEQ ID NO: 13;
for the SlugCas9 protein, the PAM is NNGG, and the target DNA sequence is represented by SEQ ID NO: 12;
for the SlutCas9 protein, the PAM is NNGR, and the target DNA sequence is represented by SEQ ID NO: 14;
for the Sa-SauriCas9 protein, the PAM is NNGG, and the target DNA sequence is represented by SEQ ID NO: 12;
for the Sa-SepCas9 protein, the PAM is NNGG, and the target DNA sequence is represented by SEQ ID NO: 12;
for the Sa-SeqCas9 protein, the PAM is NNGRM, and the target DNA sequence is represented by SEQ ID NO: 13;
for the Sa-ShaCas9 protein, the PAM is NNGRM, and the target DNA sequence is represented by SEQ ID NO: 13;
for the Sa-SlugCas9 protein, the PAM is NNGG, and the target DNA sequence is represented by SEQ ID NO: 12;
for the Sa-SlutCas9 protein, the PAM is NNGRR, and the target DNA sequence is represented by SEQ ID NO: 15; and
for the SlugCas9-HF protein, the PAM is NNGG, and the target DNA sequence is represented by SEQ ID NO: 12.

9. The CRISPR/Cas9 system for gene editing according to claim 1, wherein the sgRNA is modified via phosphorylation, shortening, lengthening, sulfurization, methylation, or hydroxylation.

10. The CRISPR/Cas9 system for gene editing according to claim 1, wherein the complex of the Cas9 protein and the sgRNA being capable of accurately locating the target DNA sequence means that the complex of the Cas9 protein and the sgRNA is capable of recognizing and binding to the target DNA sequence, or that the complex of the Cas9 protein and the sgRNA is capable of carrying a further protein fused with the Cas9 protein or a protein that specifically recognizes the sgRNA to the place where the target DNA sequence is located.

11. The CRISPR/Cas9 system for gene editing according to claim 10, wherein the complex of the Cas9 protein and the sgRNA, or the further protein fused with the Cas9 protein, or the protein that specifically recognizes the sgRNA is capable of making modification and regulation to the target DNA region, and the modification and regulation comprises regulation of gene transcription level, DNA methylation regulation, DNA acetylation modification, histone acetylation modification, single-base conversion or chromatin imaging tracking.

12. The CRISPR/Cas9 system for gene editing according to claim 1, wherein the complex of the SlugCas9-HF protein and the sgRNA is essentially incapable of or is incapable of recognizing and binding to the non-target DNA sequence, or the complex of the Cas9 protein and the sgRNA is essentially incapable of or is incapable of carrying the further protein fused with the SlugCas9-HF protein or the protein that specifically recognizes the sgRNA to the place where the non-target DNA sequence is located, and wherein the complex of the SlugCas9-HF protein and the sgRNA, or the further protein fused with the SlugCas9-HF protein, or the protein that specifically recognizes the sgRNA is essentially incapable of or is incapable of making modification and regulation to a non-target DNA region, and the modification and regulation comprises regulation of gene transcription level, DNA methylation regulation, DNA acetylation modification, histone acetylation modification, single-base conversion or chromatin imaging tracking.

13. (canceled)

14. The CRISPR/Cas9 system for gene editing according to claim 12, wherein the single-base conversion comprises conversion of adenine to guanine, or conversion of cytosine to thymine, or conversion of cytosine to uracil, or conversion between other bases.

15. A method for gene editing in cells with the CRISPR/Cas9 system for gene editing according to claim 1, wherein the method edits a target DNA sequence by recognizing and locating the target DNA sequence with a complex of a Cas9 protein and a sgRNA, and the method comprises the steps of:
(1) synthesizing a Cas9 gene sequence and cloning it into an expression vector such as pAAV2_ITR, to obtain an expression vector cloned with the Cas9 gene sequence, such as pAAV2_Cas9_ITR, wherein the Cas9 gene sequence:
(a) has a nucleotide sequence encoding an amino acid sequence represented by any one of SEQ ID NOs: 1-10 and SEQ ID NO: 58; and
(b) has a nucleotide sequence encoding an amino acid sequence that is at least 80% identical to the amino acid sequence represented by any one of SEQ ID NOs: 1-10 and SEQ ID NO: 58; or
(c) is a humanized Cas9 gene sequence, for example, the one having a nucleotide sequence represented by any one of SEQ ID NOs: 23-32 and SEQ ID NO: 112;
(2) synthesizing oligo single-stranded DNAs corresponding to the sgRNA, i.e., an oligo forward-strand sequence and an oligo reverse-strand sequence, and annealing and ligating the oligo forward-strand sequence and the oligo reverse-strand sequence to a restriction site of the expression vector cloned with the Cas9 gene sequence, such as the BsaI digestion site of plasmid pAAV2_Cas9_U6_BsaI, to obtain an expression vector, such as pAAV2_Cas9-hU6-sgRNA, for expressing the Cas9 protein and the sgRNA; wherein the sgRNA has a nucleotide sequence represented by SEQ ID NO: 11, or a nucleotide sequence that is at least 80% identical to the nucleotide sequence represented by SEQ ID NO: 11, or a modification comprising, for example, phosphorylation, shortening, lengthening, sulfurization, methylation or hydroxylation, based on the nucleotide sequence represented by SEQ ID NO: 11;
(3) delivering the expression vector expressing the Cas9 protein and the sgRNA to cells comprising a target site to edit the target site; and
(4) optionally detecting editing efficiency towards the target site by PCR amplification on the edited target site followed by T7EI digestion or next-generation sequencing.

16. The method according to claim 15, wherein the pAAV2_Cas9-hU6-sgRNA is an adeno-associated virus backbone plasmid, comprising AAV2 ITR, a CMV enhancer, a CMV promoter, SV40 NLS, Cas9, nucleoplasmin NLS, 3×HA, bGH poly(A), a human U6 promoter, a BsaI endonuclease site, and a sgRNA scaffold sequence.

17. The method according to claim 15, wherein the CRISPR/Cas9 system delivered to the cell comprises: a plasmid, a retrovirus, an adenovirus, or an adeno-associated virus vector expressing the Cas9 protein and the sgRNA; or the sgRNA and the Cas9 protein.

18. The method according to claim 15, wherein, for other Cas9 genes than the SlugCas9-HF gene, the oligo forward-strand sequence and the oligo reverse-strand sequence have the nucleotide sequences represented by SEQ ID NO: 16 and SEQ ID NO: 17, respectively; for the SlugCas9-HF gene, the oligo forward-strand sequence and the oligo reverse-strand sequence comprise a first oligo forward-strand sequence and a first oligo reverse-strand sequence represented by SEQ ID NO: 59 and SEQ ID NO: 60, respectively, and a second oligo forward-strand sequence and a second oligo reverse-strand sequence represented by SEQ ID NO: 61 and SEQ ID NO: 62, respectively.

19. The method according to claim 15, wherein, for other Cas9 genes than the SlugCas9-HF gene, the target site in the cell in step (3) has a nucleotide sequence represented by SEQ ID NO: 18; and for the SlugCas9-HF gene, the target site in the cell in step (3) has nucleotide sequences represented by SEQ ID NO: 63 and SEQ ID NO: 64, respectively.

20. The method according to claim 15, wherein the PCR template in step (4) is an edited DNA; for other Cas9 genes than the SlugCas9-HF gene, the primer sequences for PCR amplification are represented by SEQ ID NO: 19, SEQ ID NO: 20, SEQ ID NO: 21, and SEQ ID NO: 22; and for the SlugCas9-HF gene, the primer sequences for PCR amplification are represented by SEQ ID NO: 65, SEQ ID NO: 66, SEQ ID NO: 21, SEQ ID NO: 22, and SEQ ID NO: 67.

21. The method according to claim 15, wherein the cell comprises eukaryotic cells and prokaryotic cells; the eukaryotic cells comprise mammalian cells and plant cells. The mammalian cells comprise Chinese hamster ovary cells, baby hamster kidney cells, mouse Sertoli cells, mouse breast tumor cells, buffalo rat hepatic cells, rat hepatoma cells, SV40-transformed monkey kidney CVI lines, monkey kidney cells, canine kidney cells, human cervical cancer cells, human lung cells, human liver cells, HIH/3T3 cells, human U2-OS osteosarcoma cells, human A549 cells, human K562 cells, human HEK293 cells, human HEK293T cells, human HCT116 cells, or human MCF-7 cells or TRI cells.

22. A kit of a CRISPR/Cas9 gene editing system for gene editing, the kit comprising:
(1) a Cas9 protein and a sgRNA, wherein the Cas9 protein is:
a SauriCas9 protein having an amino acid sequence represented by SEQ ID NO: 1,
a ShaCas9 protein having an amino acid sequence represented by SEQ ID NO: 2,
a SlugCas9 protein having an amino acid sequence represented by SEQ ID NO: 3,
a SlutCas9 protein having an amino acid sequence represented by SEQ ID NO: 4,
a Sa-SauriCas9 protein having an amino acid sequence represented by SEQ ID NO: 5,
a Sa-SepCas9 protein having an amino acid sequence represented by SEQ ID NO: 6,
a Sa-SeqCas9 protein having an amino acid sequence represented by SEQ ID NO: 7,
a Sa-ShaCas9 protein having an amino acid sequence represented by SEQ ID NO: 8,
a Sa-SlugCas9 protein having an amino acid sequence represented by SEQ ID NO: 9,
a Sa-SlutCas9 protein having an amino acid sequence represented by SEQ ID NO: 10,
a SlugCas9-HF protein having an amino acid sequence represented by SEQ ID NO: 58, or
a Cas9 protein having an amino acid sequence that is at least 80% identical to the amino acid sequence represented by any one of SEQ ID NOs: 1-10 and SEQ ID NO: 58; and
the sgRNA has a nucleotide sequence represented by SEQ ID NO: 11, or is a modified sgRNA sequence based on SEQ ID NO: 11; or
(2) an expression vector cloned with a Cas9 gene sequence and an oligo single-stranded DNA corresponding to a sgRNA, wherein
the Cas9 gene sequence:
(a) has a nucleotide sequence encoding an amino acid sequence represented by any one of SEQ ID NO: 1-10 and SEQ ID NO: 58; and
(b) has a nucleotide sequence encoding an amino acid sequence that is at least 80% identical to the amino acid sequence represented by any one of SEQ ID NO: 1-10 and SEQ ID NO: 58; or
(c) is a humanized Cas9 gene sequence, for example, the one having a nucleotide sequence represented by any one of SEQ ID NOs: 23-32 and SEQ ID NO: 112; and
the sgRNA has a nucleotide sequence represented by SEQ ID NO: 11, or a modified sgRNA sequence based on SEQ ID NO: 11.

23. Use of the CRISPR/Cas9 system for gene editing according to claim 1 in gene knockout, site-directed base change, site-directed insertion, regulation of gene transcription level, regulation of DNA methylation, modification of DNA acetylation, modification of histone acetylation, single-base conversion or chromatin imaging tracking.

24. The CRISPR/Cas9 system for gene editing according to claim 11, wherein the single-base conversion comprises conversion of adenine to guanine, or conversion of cytosine to thymine, or conversion of cytosine to uracil, or conversion between other bases.
